(19) United States
(12) Patent Application Publication
(10) Pub. No.: US 2006/0064775 A1
(43) Pub. Date: Mar. 23, 2006

(54) Method for increasing resistance against stress factors in plants

(75) Inventors: Markus Frank, Mannheim (DE); Karl-Heinz Kogel, Lollar (DE); Ralph Huckelhoven, Gieben (DE)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 10/548,748

(22) PCT Filed: Mar. 10, 2004
(86) PCT No.: PCT/EP04/02436

§ 371 Date: Sep. 8, 2005

(30) Foreign Application Priority Data
Mar. 12, 2003 (DE) 103 11 118.2

(51) Int. Cl. A01H 1/00; C12N 15/87

(52) U.S. Cl. 800/279

(57) ABSTRACT

The invention relates to methods for generating or increasing the resistance, in plants, to at least one biotic or abiotic stress factor, preferably to plant pathogens, by increasing the expression of at least one Bax inhibitor 1 (BI1) protein in at least one plant tissue, with the proviso that the expression in the leaf epidermis remains essentially unchanged. The invention furthermore relates to recombinant expression cassettes and vectors which comprise a nucleic acid sequence coding for a BI protein under the control of a tissue-specific promoter, the promoter having essentially no activity in the leaf epidermis. The invention furthermore relates to recombinant plants transformed with said expression cassettes or vectors, to cultures, parts or recombinant propagation material derived from these plants, and to the use of same for the production of foodstuffs, feeding stuffs, seed, pharmaceuticals or fine chemicals.

The invention relates to methods for generating or increasing the resistance, in plants, to at least one biotic or abiotic stress factor, preferably to plant pathogens, by increasing the expression of at least one Bax inhibitor 1 (BI1) protein in at least one plant tissue, with the proviso that the expression in the leaf epidermis remains essentially unchanged. The invention furthermore relates to recombinant expression cassettes and vectors which comprise a nucleic acid sequence coding for a BI protein under the control of a tissue-specific promoter, the promoter having essentially no activity in the leaf epidermis. The invention furthermore relates to recombinant plants transformed with said expression cassettes or vectors, to cultures, parts or recombinant propagation material derived from these plants, and to the use of same for the production of foodstuffs, feeding stuffs, seed, pharmaceuticals or fine chemicals.

The aim of plant biotechnology work is the generation of plants with advantageous novel properties, for example for increasing agricultural productivity, increasing the quality in the case of foodstuffs, or for producing specific chemicals or pharmaceuticals. The plant's natural defense mechanisms against pathogens are frequently insufficient. Fungal diseases alone result in annual yield losses of many billions of US$. The introduction of foreign genes from plants, animals or microbial sources can increase the defenses. Examples are the protection of tobacco against feeding damage by insects by expressing Bacillus thuringiensis endotoxins (Vaeck et al. (1987) Nature 328:33-37) or by protecting tobacco against fungal disease by expressing a bean chitinase (Broglie et al. (1991) Science 254: 1194-1197). However, most of the approaches described only offer resistance to a single pathogen or a narrow spectrum of pathogens.

Only a few approaches exist which confer, to plants, a resistance to a broader spectrum of pathogens, in particular fungal pathogens. Systemic acquired resistance (SAR)— a defense mechanism in a variety of plant/pathogen interactions—can be conferred by application of endogenous messenger substances such as jasmonic acid (JA) or salicylic acid (SA) (Ward et al. (1991) Plant Cell 3:1085-1094; Uknes et al. (1992) Plant Cell 4(6):645-656). Similar effects can also be achieved by synthetic compounds such as 2,6-dichloroisonicotinic acid (DCINA) or S-methyl benzo[1,2,3)thiadiazol-7-thiocarboxylate (BTH; Bion®) (Friedrich et al. (1996) Plant J 10(1):61-70; Lawton et al. (1996) Plant J 10:71-82). The expression of the pathogenesis-related (PR) proteins, which are upregulated in the case of SAR, may also cause pathogen resistance in some cases.

In barley, the Mlo locus is described as a negative regulator of pathogen defense. The loss or loss of function, of the Mlo gene causes an increased, race-unspecific resistance against a large number of mildew isolates (Büschges R et al. (1997) Cell 88:695-705; Jorgensen J H (1977) Euphytica 26:55-62; Lyngkjaer M F et al. (1995) Plant Pathol 44:786-790).

The Mlo gene is described (Büschges R et al. (1997) Cell 88:695-705; WO 98/04586; Schulze-Lefert P, Vogel J (2000) Trends Plant Sci. 5:343-348). Various Mlo homologs from other cereal species have been isolated. Methods using these genes for obtaining pathogen resistance have been described (WO 98/04586; WO 00/01722; WO 99/47552). The disadvantage is that Mlo-deficient plants also initiate the abovementioned defense mechanisms in the absence of a pathogen, which manifests itself in a spontaneous dying of leaf cells (Wolter M et al. (1993) Mol Gen Genet 239:122-128). This is why mlo-resistant plants suffer a yield loss of approximately 5% (Jörgensen J H (1992) Euphytica 63: 141-152). Furthermore, the spontaneous dying of the leaf cells brings about a disadvantageous hypersusceptibility to necrotrophic and hemibiotrophic pathogens such as Magnaporte grisea (M. grisea) or Cochliobolus sativus (Bipolaris sorokiniana) (Jarosch B et al. (1999) Mol Plant Microbe Interact 12:508-514; Kumar J et al. (2001) Phytopathology 91:127-133).

Factors which mediate an effect against necrotrophic fungi which can be compared with the mlo resistance have not been identified to date. The reason for this may be the specific infection mechanism of the necrotrophic fungi: instead of an appressoria-mediated penetration, they first release mycotoxins and enzymes into the plant host cell, which leads to the death of the cell. Only then is the cell penetrated (Shirasu K and Schulze-Lefert P (2000) Plant Mol Biol 44:371-385). Similar infection strategies are employed by bacterial pathogens such as Erwinia carotovora (Whitehead N A et al. (2002) Antonie van Leeuwenhoek 81: 223-231). Penetration resistance with the aid of the formation of papillae is no efficient defense strategy in such a case.

Apoptosis, also referred to as programmed cell death, is an essential mechanism for maintaining tissue homeostasis, and thus constitutes a negatively regulating mechanism which counteracts cell division. In multicelled organisms, apoptosis is a natural part of ontogenesis and involves, inter alia, the development of the organs and the removal of senescent, infected or mutated cells. Apoptosis allows efficient elimination of undesired cells to take place. Interference with, or inhibition of, apoptosis contributes to the pathogenesis of a range of diseases, inter alia carcinogenesis. The main effectors of apoptosis are aspartate-specific cysteine proteases, known as caspases. They can be activated by means of at least two apoptotic signal pathways: firstly, the activation of the TNF (tumor necrosis factor) receptor family; secondly, the central role played by mitochondria. The activation of the mitochondrial apoptotic signal pathway is regulated by proteins of the Bcl-2 family. This protein family consists of antiapoptotic and proapoptotic proteins such as, for example, Bax. In the case of an apoptotic stimulus, the Bax protein undergoes an allosteric conformational change, which leads to the protein being anchored in the mitochondrial external membrane and in its oligomerization. The result of these oligomers is that proapoptotic molecules are released from the mitochondria into the zytosol, and these molecules bring about an apoptotic signal cascade and, eventually, the degradation of specific cellular substrates, which results in the death of the cell. The Bax inhibitor 1 BI1 has been isolated via its property to inhibit the proapoptotic effect of BAX (Xu Q & Reed J C (1998) Mol Cell 1(3): 337-346). BI1 is a highly conserved protein. It is predominantly found as integral constituent of intracellular membranes. BI1 interacts with bcl-2 and bcl-x1. The overexpression of BI1 in mammalian cells suppresses the proapoptotic effect of BAX, etoposide and staurosporin, but not of Fas antigen (Roth W and Reed J C (2002) Nat Med 8: 216-218). In contrast, the inhibition of BI1 by antisense RNA induces apoptosis (Xu Q & Reed J C (1998) Mol Cell 1(3):337-346). The first plant homologs of BI1 have been isolated from rice and Arabidopsis (Kawai et al. (1999) FEBS Lett 464:143-147; Sanchez et al (2000) Plant J 21:393-399). These plant proteins suppress the BAX-induced cell death in yeast. The amino acid sequence homology with human BI1 amounts to approximately 45%. The Arabidopsis homolog AtBI1 is capable of suppressing, in recombinant plants, the pro-apoptotic effect of murine BAX (Kawai-Yamada et al. (2001) Proc Natl Acad Sci USA 98(21):12295-12300). The rice (Oryza sativa) BI1 homolog OsBI1 is expressed in all plant tissues (Kawai et al. (1999) FEBS Lett 464: 143-147). Furthermore described are BI1 genes from barley (Hordeum vulgare; GenBank Acc. No.: AJ290421), rice (GenBank Acc. No.: AB025926), Arabidopsis (GenBank Acc. No.: AB025927), tobacco (GenBank Acc. No.: AF390556) and oilseed rape (GenBank Acc. No.: AF390555, Bolduc N et al. (2003) Planta 216:377-386). The expression of BI1 in barley is upregulated as the result of infection with mildew (Hückelhoven R et al. (2001) Plant Mol Biol 47(6):739-748).

WO 00/26391 describes the overexpression of the anti-apoptotic genes Ced-9 from C. elegans, sfIAP from Spodoptera frugiperda, bcl-2 from human and bcl-x1 from chicken in plants for increasing the resistance to necrotrophic or hemibiotrophic fungi. Plant BI1 homologs are not disclosed. The expression is under the control of constitutive promoters. Furthermore described is the expression of a BI1 protein from Arabidopsis under the strong constitutive 35S CaMV promoter in rice cells, and a resistance, induced thereby, to cell-death-inducing substances from Magnaporthe grisea (Matsumura H et al. (2003) Plant J 33:425-434).

Surprisingly, it has been found within the scope of the present invention that, while constitutive expression of a BI1 protein brings about resistance to necrotrophic fungi, the result is the breaking of the mlo-mediated resistance to the obligate-biotrophic Powdery Mildew (see comparative experiment 1). This questions the economical use of the methods described in the prior art.

The object was to provide plant pathogen defense methods which make possible an efficient defense against plant pathogens (preferably necrotrophic pathogens) without breaking any other existing resistance to other pathogens (such as, for example, biotrophic pathogens). This object is achieved by the method according to the invention.

A first subject of the invention relates to methods for generating or increasing the resistance, in plants, to at least one biotic or abiotic stress factor, comprising the following steps:
a) increasing the amount of protein, or the function, of at least one Bax inhibitor-1 (BI1) protein in at least one plant tissue with the proviso that the expression in the leaf epidermis remains essentially unchanged or is reduced, and
b) selection of the plants in which, in comparison with the starting plant, a resistance to at least one biotic or abiotic stress factor exists or is increased.

The biotic or abiotic stress factor is preferably a pathogen, especially preferably a pathogen selected from the group of the necrotrophic and hemibiotrophic pathogens.

By epidermis, the skilled worker means the predominant epidermal tissue of primary aerial plant parts, for example of the shoot, the leaves, flowers, fruits and seeds. The epidermal cells secrete outwardly a water-repellent layer, the cuticle. The roots are surrounded by the rhizodermis, which, in many ways, resembles the epidermis, but also shows pronounced differences. While the outermost layer of the apical meristem gives rise to the epidermis, the formation of the rhizodermis is much less clear. Depending on the species, it can be considered, in phylogenetic terms, either as part of the calyptra or as part of the primary cortex. The epidermis has a number of functions: it protects the plant against desiccation and regulates the transpiration rate. It protects the plant against a wide range of chemical and physical external influences, against being fed upon by animals and against attack by parasites. It is involved in gas exchange, in the secretion of certain metabolites and in the absorption of water. It comprises receptors for light and mechanical stimuli. It thus acts as signal transformer between the environment and the plant. In accordance with its various functions, the epidermis comprises a number of differently differentiated cells. To this must be added species-specific variants and different organizations of the epidermides in the individual parts of a plant. Essentially, it consists of three categories of cells: the “actual” epidermal cells, the cells of the stomata and of the trichomes (Greek: Trichoma, hair), epidermal appendages of varying shape, structure and function. The “actual”, i.e. the least specialized epidermal cells, account for most of the bulk of the cells of the epidermal tissue. In topview, they appear either polygonal (slab or plate shaped) or elongated. The walls between them are often wavy or sinuate. It is not known what induces this shape during development; existing hypotheses only offer unsatisfactory explanations herefor. Elongated epidermal cells can be found in organs or parts of organs that are elongated themselves, thus, for example, in stems, petioles, leaf veins and on the leaves of most monocots. The upper surface and undersurface of laminae can be covered in epidermides with different structures, it being possible for the shape of the cells, the wall thickness and the distribution and number of specialized cells (stomata and/or trichomes) per unit area to vary. A high degree of variation is also found within individual families, for example in the Crassulaceae. In most cases, the epidermis consists of a single layer, though multi-layered water-storing epidermides have been found among species from a plurality of families (Moraceae: most Ficus species; Piperaceae: Peperonia, Begoniaceae, Malvaceae and the like). Epidermal cells however secrete a cuticle on the outside which covers all epidermal surfaces as an uninterrupted film. It may either be smooth or structured by bulges, rods, folds and furrows. However, the folding of the cuticle, which can be observed when viewing the surface, is not always caused by cuticular rods. Indeed, there are cases where cuticular folding is merely the expression of the underlying bulges of the cell wall. Epidermal appendages of various form, structure and function are referred to as trichomes and, in the present context, likewise come under the term “epidermis”. They occur in the form of protective hairs, supportive hairs and gland hairs in the form of scales, different papillae and, in the case of roots, as absorbent hairs. They are formed exclusively by epidermal cells. Frequently, a trichome is formed by only one such a cell, however, occasionally, more than one cell is involved in its formation.

The term “epidermis” likewise comprises papillae. Papillae are bulges of the epidermal surface. The textbook example are the papillae on flower surfaces of pansy (Viola tricolor) and the leaf surfaces of many species from tropical rain forests. They impart a velvet-like consistency to the surface. Some epidermal cells can form water stores. A typical example are the water vesicles at the surfaces of many Mesembryanthemum species and other succulents. In some plants, for example in the case of campanula (Campanula persicifolia), the outer walls of the epidermis are thickened like a lens.

The main biomass of all tissues is the parenchyma. The parenchymatic tissues include the mesophyll which, in leaves, can be differentiated into palisade parenchyma and spongy parenchyma.

Accordingly the skilled worker understands, by mesophyll, a parenchymatic tissue. Parenchymatic cells are always alive, in most cases isodiametric, rarely elongated. The pith of the shoots, the storage tissues of the fruits, seeds, the root and other underground organs are also parenchymas, as is the mesophyll.

In the leaves of most ferns and phanerogams, especially in the case of the dicotts and many monocotts, the mesophyll is subdivided into palisade parenchymas and spongy parenchymas. A “typical” leaf is of dorsiventral organization. In most cases, the palisade parenchyma is at the upper surface of the leaf immediately underneath the epidermis. The sponge parenchyma fills the underlying space. It is interspersed by a voluminous intercellular system whose gas space is in direct contact with the external space via the stomata.

The palisade parenchyma consists of elongated cylindrical cells. In some species, the cells are irregular, occasionally bifurcate (Y-shaped: arm palisade parenchyma). Such variants are found in ferns, conifers and a few angiosperms (for example in some Ranunculaceae and Caprifoliaceae species [example: elder]). Besides the widest-spread organization form which has just been described, the following variants have been found: palisade parenchyma at the leaf undersurface. Particularly conspicuously in scaly leaves. (for example arbor vitae (thuja), and in the leaves of wild garlic (Allium ursinum). Palisade parenchyma at both leaf surfaces (upper surface and undersurface). Frequently found in plants of dry habitats (xerophytes). Example: prickly lettuce (Lactuca serriola); ring-shaped closed palisade parenchyma: in cylindrically organized leaves and in conifers' needles.

The variability of the cells of the spongy parenchyma, and the organization of the spongy parenchyma itself, are even more varied than that of the palisade parenchyma. It is most frequently referred to as aerenchyma since it comprises a multiplicity of interconnected intercellular spaces. The mesophyll may comprise what is known as the assimilation tissue, but the terms mesophyll and assimilation tissue are not to be used synonymously. There are chloroplast-free leaves whose organization differs only to a minor extent from comparable green leaves. As a consequence, they comprise mesophyll, but assimilation does not take place; conversely, assimilation also takes place in, for example, sections of the shoot. Further aids for characterizing epidermis and mesophyll can be found by the skilled worker for example in v. GUTTENBERG, H.: Lehrbuch der Allgemeinen Botanik [Textbook of general botany]. Berlin: Akademie-Verlag 1955 (5th Ed.), HABERLANDT, G.: Physiologische Pflanzenanatomie [Physiological plant anatomy]. Leipzig: W. Engelmann 1924 (6th Ed.); TROLL, W.: Morphologie der Pflanzen [Plant morphology]. Volume 1: Vegetationsorgane [Vegetation organs]. Berlin: Gebr. Borntraeger, 1937; TROLL, W.: Praktische Einführung in die Pflanzenmorphologie [Practical introduction to plant morphology]. Jena: VEB G. Thieme Verlag 1954/1957; TROLL, W., HÖHN, K.: Allgemeine Botanik [General botany]. Stuttgart: F. Enke Verlag, 1973 (4th Ed.)

In one embodiment, the epidermis is characterized in biochemical terms. In one embodiment, the epidermis can be characterized by the activity of one or more of the following promoters:
WIR5 (=GstA1), acc. X56012, Dudler & Schweizer, unpublished.
GLP4, acc. AJ310534; Wei, Y.; Zhang, Z.; Andersen, C. H.; Schmelzer, E.; Gregersen, P. L.; Collinge, D. B.; Smedegaard-Petersen, V.; Thordal-Christensen, H. (1998) An epidermis/-papilla-specific oxalate oxidase-like protein in the defence response of barley attacked by the powdery mildew fungus. Plant Molecular Biology 36, 101-112.
GLP2a, acc. AJ237942, Schweizer, P., Christoffel, A. and Dudler, R. (1999). Transient expression of members of the germin-like gene family in epidermal cells of wheat confers disease resistance, Plant J 20, 541-552.
Prx7, acc. AJ003141, Kristensen B K, Ammitzböll H, Rasmussen S K & Nielsen K A. 2001. Transient expression of a vacuolar peroxidase increases susceptibility of epidermal barley cells to powdery mildew. Molecular Plant Pathology, 2(6), 311-317
GerA, acc. AF250933; Wu S, Druka A, Horvath H, Kleinhofs A, Kannangara G & von Wettstein D, 2000. Functional characterization of seed coat-specific members of the barley germin gene family. Plant Phys Biochem 38, 685-698
OsROC1, acc. AP004656
RTBV, acc. AAV62708, AAV62707; Klöti, A, Henrich C, Bieri S, He X, Chen G, Burkhardt P K, Wünn J, Lucca, P, Hohn, T, Potrykus I & Fütterer J, 1999, Upstream and downstream sequence elements determine the specificity of the rice tungro bacilliform virus promoter and influence RNA production after transcription initiation. PMB 40, 249-266

In one embodiment, the epidermis comprises the fact that all the abovementioned promoters are active in the tissue or the cell. In another embodiment, the epidermis comprises the fact that only some of the promoters are active, for example 2, 3, 5 or 7 or more, but at least from only one of those detailed above.

In one embodiment, the mesophyll is characterized in biochemical terms. In one embodiment, the mesophyll can be characterized by the activity of one or more of the following promoters:
PPCZm1 (=PEPC); Kausch, A. P., Owen, T. P., Zachwieja, S. J., Flynn, A. R. and Sheen, J. (2001) Mesophyll-specific, light and metabolic regulation of the C(4)PPCZm1 promoter in transgenic maize. Plant Mol. Biol. 45, 1-15
OsrbcS, Kyozuka et al PlaNT Phys: 1993 102: Kyozuka J, McElroy D, Hayakawa T, Xie Y, Wu R & Shimamoto K. 1993. Light-regulated and cell-specific expression of tomato rbcs-gusA and rice rbcs-gusA fusion genes in transgenic rice. Plant Phys 102, 991-1000
OsPPDK, acc. AC099041, unpublished.
TaGF-2.8, acc. M63223; Schweizer, P., Christoffel, A. and Dudler, R. (1999). Transient expression of members of the germin-like gene family in epidermal cells of wheat confers disease resistance, Plant J 20, 541-552.
TaFBPase, acc. X53957 unpublished.
TaWIS1, acc. AF467542; U.S. 200220115849
HvBIS1, acc. AF467539; U.S. 200220115849
ZmMIS1, acc. AF467514; U.S. 200220115849
HvPR1a, acc. X74939; Bryngelsson et al. Molecular Plant-Microbe Interactions (1994)
HvPR1b, acc. X74940; Bryngelsson et al. Molecular Plant-Microbe Interactions (1994)
HvB1, 3gluc; acc. AF479647; unpublished.
HvPrx8, acc. AJ276227; Kristensen et al MPP 2001 (see above)
HvPAL, acc. X97313; Wei, Y.; Zhang, Z.; Andersen, C. H.; Schmelzer, E.; Gregersen, P. L.; Collinge, D. B.; Smedegaard-Petersen, V.; Thordal-Christensen, H. (1998) An epidermis/-papilla-specific oxalate oxidase-like protein in the defence response of barley attacked by the powdery mildew fungus. Plant Molecular Biology 36, 101-112.

In one embodiment, the mesophyll comprises the fact that all the abovementioned promoters are active in the tissue or the cell. In another embodiment, the mesophyll comprises the fact that only some of the promoters are active, for example 2, 3, 5 or 7 or more, but at least from only one of those detailed above.

In one embodiment, all of the abovementioned promoters are active in a plant used or produced in accordance with the invention or in the epidermis and in the mesophyll in a plant according to the invention. In one embodiment, only some of the abovementioned promoters are active, for example 2, 5, 7 or more; however, at least one of the promoters detailed above is active in each case.

In a preferred embodiment, the increase in the protein quantity or function of the BI1 protein takes place in a root-, tuber- or mesophyll-specific manner, especially preferably in a mesophyll-specific manner, for example by recombinant expression of a nucleic acid sequence coding for said BI1 protein under the control of a root-, tuber- or mesophyll-specific promoter, preferably under the control of a mesophyll-specific promoter.

As described in the present text, in one embodiment, the expression or function, in the mesophyll of a plant, of the protein according to the invention or of the BI-1 characterized in the present text is increased. An increase in expression can be achieved as described hereinbelow. By increased expression or function, the present text means both the activation or enhancement of the expression or function of the endogenous protein including a de novo expression, but also an increase in or enhancement as the result of the expression of a transgenic protein or factor.

In an especially preferred embodiment, the increase in the protein quantity or function of at least one plant BI1 protein can be combined with an mlo-resistant phenotype or with the inhibition or reduction, in comparison with a control plant, of the expression of MLO, RacB and/or NaOx in the plant or a part thereof, for example in a tissue, but especially advantageously at least in the epidermis or a considerable number of the epidermal cells and/or with the increase in the expression or function of PEN2 and/or PEN1 in the plant, for example constitutively, or a part thereof, for example in a tissue, but especially advantageously at least in the epidermis or in a considerable number of the epidermal cells, with the proviso that the expression of a plant BI1 protein in the leaf epidermis remains essentially unchanged or is reduced, thus providing a combined resistance to both necrotrophy and biotrophic pathogens.

The Mlo locus has been described in barley as negative regulator of pathogen defense. The loss, or loss of function, of the Mlo gene brings about an increased, race-unspecific resistance to a number of mildew isolates (Büschges R et al. (1997) Cell 88:695-705; Jorgensen J H (1977) Euphytica 26:55-62; Lyngkjaer M F et al. (1995) Plant Pathol 44:786-790).

The Mlo gene has been described (Büschges R et al. (1997) Cell 88:695-705; WO 98/04586; Schulze-Lefert P, Vogel J (2000) Trends Plant Sci. 5:343-348). Various Mlo homologs from other cereal species have been isolated.

An mlo-resistant phenotype can be obtained as described in the prior art. Methods using these genes for obtaining a pathogen resistance are described, inter alia, in WO 98/04586; WO 00/01722; WO 99/47552.

In one embodiment of the present invention, the activity, expression or function of MLO, RacB and/or NaOx in the plant or a part thereof, for example in a tissue, but especially advantageously at least in the epidermis or a substantial number of epidermal cells can advantageously be inhibited or reduced in comparison with a control plant or a part thereof. By reducing the activity or function of MLO, RacB and/or NaOx in the plant or a part thereof, for example in a tissue, but especially advantageously at least in the epidermis or a substantial number of epidermal cells, it is preferred to increase the resistance, or withstanding power, to biotrophic pathogens in plants produced in accordance with the invention. This is especially advantageous in combination with a reduction or suppression of cell death due to necrosis. The activity or function of MLO, RacB and/or NaOx can be reduced or inhibited analogously to what has been described for MLO in WO 98/04586; WO 00/01722; WO 99/47552 and the other publications mentioned hereinbelow, whose content is herewith expressly incorporated into the present description, in particular for describing the activity and inhibition of MLO. The description of the abovementioned publications describes processes, methods and especially preferred embodiments for reducing or inhibiting the activity or function of MLO; the examples detail specifically how this can be performed.

The reduction of the activity or function, if appropriate the expression, of RacB is described in detail in WO 2003020939, which is herewith expressly incorporated into the present description. The description of the abovementioned publication describes processes and methods for reducing or inhibiting the activity or function of BI-1; the examples detail specifically how this can be performed. It is especially preferred to carry out the reduction or inhibition of the activity or function of RacB as described in the embodiments and the examples which are especially preferred in WO 2003020939 and in the organisms specified therein as being especially preferred, in particular in a plant or a part thereof, for example in a tissue, but especially advantageously at least in the epidermis or a substantial number of epidermal cells. The reduction of the activity or function, if appropriate the expression, of RacB is described in detail in WO 2003020939. In WO 2003020939, the skilled worker can find the sequences which code for RacB proteins and can also identify RacB by means of the method provided in WO 2003020939.

The reduction of the activity or function, if appropriate of the expression, of NaOX is described in detail in PCT/EP/03/07589 which is herewith expressly incorporated into the present description. The description of the abovementioned publication describes processes and methods for reducing or inhibiting the activity or function of NaOx; the examples detail specifically how this can be performed. It is especially preferred to carry out the reduction or inhibition of the activity or function of NaOx as described in the embodiments and the examples which are especially preferred in PCT/EP/03/07589 and in the organisms specified therein as being especially preferred, in particular in a plant or a part thereof, for example in a tissue, but especially advantageously at least in the epidermis or a substantial number of epidermal cells. In PCT/EP/03/07589, the skilled worker can find the sequences which code for NaOx proteins and can also identify NaOx by means of the method provided in PCT/EP/03/07589.

In one embodiment of the present invention, the activity, expression or function of PEN1, PEN2 and/or SNAP34 can advantageously be increased in the plant, for example constitutively, or in a part thereof, for example in a tissue, but especially advantageously at least in the epidermis or a substantial number of epidermal cells. The increase in activity, which also comprises a de novo expression, of PEN1, PEN2 and/or SNAP 34 in the plant, for example constitutively, or in a part thereof, for example in a tissue, but especially advantageously at least in the epidermis or a substantial number of epidermal cells will preferably increase the resistance or withstanding power to biotrophic pathogens in the plants produced in accordance with the invention. This is especially advantageous in combination with a reduction or suppression of cell death due to necrosis. The increase in the activity or function, if appropriate the expression, of PEN2 is described in detail in WO03074688, which is herewith expressly incorporated into the present description. The description of the abovementioned publications describes processes and methods for reducing or inhibiting the activity or function of PEN2; the examples detail specifically how this can be performed. The reduction or inhibition of the activity or function of PEN2 is especially preferably carried out in accordance with the embodiments and examples which are especially preferred in WO03074688 and in the organisms detailed therein as being especially preferred, in particular in plants, for example constitutively, or in a part thereof, for example in a tissue, but especially advantageously at least in the epidermis or a considerable part of the epidermal cells. In WO03074688, the skilled worker will find the sequences which code for PEN2 proteins and can also identify PEN2 by means of the method provided in WO03074688.

The expression of PEN1 and SNAP34 can be increased analogously to the methods described in WO03074688. Owing to his general expert knowledge and the prior art with which he is familiar, the skilled worker can isolate and overexpress PEN1 and SNAP34 nucleic acid sequences and protein sequences. SEQ ID NO: 39 describes the nucleic acid sequence which codes for PEN1 from barley; the protein sequence is described in SEQ ID No: 40.

SEQ ID NO: 41 describes the nucleic acid sequence which codes for PEN1 from Arabidopsis thaliana; the protein sequence is described in SEQ ID NO: 42. PEN1 from Arabidopsis thaliana is published under the accession numbers NM 202559 and NM 112015. The homolog from barley is disclosed in accession numbers AY246907 and AY246906 as ROR2. They are members of the fairly large family of the syntaxin proteins. Thus, the skilled worker can use simple homology comparisons for identifying further syntaxin proteins which are expressed as potential resistance genes in the method according to the invention.

SEQ ID NO: 43 describes the nucleic acid sequence which codes for SNAP34 from barley; the protein sequence is described in SEQ ID NO: 44. The SNAP-34 homolog from barley is also published as AY 247208 (SNAP-34). Homologs whose function is unknown and which might play a role in the resistance are published as AY 247209 (SNAP-28) and AY 247210 (SNAP-25). The following Arabidopsis genes show a higher degree of homology with barley SNAP34 than barley SNAP-28 or SNAP-25 to SNAP-34 and can thus advantageously be co-overexpressed as potential resistance-mediating genes:
AAM 62553-Arabidopsis SNAP25a
NP 200929-Arabidopsis SNAP33b
NP 172842-Arabidopsis SNAP30
NP 196405-Arabidopsis SNAP29

Accordingly, the invention also relates to a plant in which a polypeptide which is encoded by a nucleic acid molecule comprising the sequences shown in SEQ. ID NO: 39, 41 or 43 or one of the sequences shown in the abovementioned database publications or which comprises one of the amino acid sequences shown in the abovementioned database publications or in SEQ. ID No.: 40, 42 or 44, or which is a functional equivalent thereof or which has at least 50%, preferably 70%, more preferably 80%, even more preferably 90% or more homology with the abovementioned sequences at the coding nucleic acid molecule level or, preferably, at the amino acid level is overexpressed at least furthermore in the epidermis, or relates to a plant in which the above-characterized polypeptide is activated, or its activity or function increased, constitutively or in a part, for example in a tissue, but especially advantageously at least in the epidermis or a substantial number of epidermal cells.

A reduction of the expression or activity can be brought about by the methods with which the skilled worker is familiar, for example mutagenesis, for example EMS, if appropriate TILLING, iRNA; ribozyme, silencing, knockout, and the like. Reduction methods are described in particular in WO 2003020939, whose methods can readily be adapted to the sequences described herein, which is why the content of WO 2003020939 is explicitly incorporated herein.

The lowering or reduction of the expression of a BI-1 protein, the BI-1 activity or the BI-1 function can be performed in many ways.

“Lowering”, “to lower”, “reduction” or “to reduce” is to be understood in the broad sense in connection with a BI-1 protein, a BI-1 activity or BI-1 function and comprises the partial or essentially complete prevention or blocking of the functionality of a BI-1 protein, as the result of different cell-biological mechanisms.

A reduction for the purposes of the invention also comprises a quantitative reduction of a BI-1 protein down to an essentially complete absence of the BI-1 protein (i.e. lacking detectability of BI-1 activity or BI-1 function or lacking immunological detectability of the BI-1 protein). In this context, the expression of a certain BI-1 protein or the BI-1 activity, or BI-1 function, in a cell or an organism is preferably reduced by more than 50%, especially preferably by more than 80%, very especially preferably by more than 90%.

The invention comprises a variety of strategies for reducing the expression of a BI-1 protein, the BI-1 activity or the BI-1 function. The skilled worker will recognize that a series of different methods is available for influencing the expression of a BI-1 protein, the BI-1 activity or the BI-1 function in the desired manner.

A reduction of the BI-1 activity or the BI-1 function is preferably achieved by reduced expression of an endogenous BI-1 protein.

A reduction of the amount of BI-1 protein, the BI-1 activity or the BI-1 function can be effected using the following methods:
a) introduction of a double-stranded BI-1 RNA nucleic acid sequence (BI-1-dsRNA), or of an expression cassette(s) which ensure(s) the expression thereof;
b) introduction of a BI-1 antisense nucleic acid sequence, or of an expression cassette which ensures the expression thereof. Comprised are those methods in which the antisense nucleic acid sequence is directed against a BI-1 gene (that is, genomic DNA sequences) or against a BI-1 gene transcript (that is, RNA sequences). Also comprised are α-anomeric nucleic acid sequences;
c) Introduction of a BI-1 antisense nucleic acid sequence in combination with a ribozyme, or of an expression cassette which ensures the expression thereof;
d) Introduction of BI-1 sense nucleic acid sequences for inducing cosuppression, or of an expression cassette which ensures the expression thereof;
e) Introduction of a nucleic acid sequence coding for dominant-negative BI-1 protein, or of an expression cassette which ensures the expression thereof;
f) Introduction of DNA- or protein-binding factors against BI-1 genes, BI-1 RNAs or BI-1 proteins, or of an expression cassette which ensures their expression;
g) Introduction of viral nucleic acid sequences and expression constructs which bring about degradation of the BI-1 RNA, or of an expression cassette which ensures the expression thereof;
h) Introduction of constructs for inducing homologous recombination at endogenous BI-1 genes, for example for the generation of knockout mutants;
i) Introduction of mutations in endogenous BI-1 genes for generating a loss of function (for example generation of stop codons, reading-frame shifts and the like),
   it being necessary for each of the abovementioned methods to be carried out in an epidermis-specific manner, i.e. the expression in the epidermal tissue remains unchanged or is reduced. In this context, each of these methods can bring about a reduction of the BI-1 expression, BI-1 activity or BI-1 function as defined in the invention. A combined use is also feasible. Further methods are known to the skilled worker and may comprise hindering or preventing the processing of the BI-1 protein, of the transport of the BI-1 protein or its mRNA, inhibition of ribosomal attachment, inhibition of RNA splicing, induction of a BI-1-RNA-degrading enzyme and/or inhibition of the elongation or termination of translation.

The epidermis-specific reduction can be effected for example by the transient application of the abovementioned methods to epidermal cells or by a specific transformation of essentially only epidermal cells or by the expressional control of the abovementioned constructs under an epidermis-specific promoter or other epidermis-specific control element.

The individual methods which are preferred shall now be described briefly in the following text:
a) Introduction of a double-stranded BI-1 RNA nucleic acid molecule (BI-1-dsRNA)

The method of regulating genes by means of double-stranded RNA (“double-stranded RNA interference”; dsRNAi) has been described repeatedly in animal and plant organisms (for example Matzke M A et al. (2000) Plant Mol Biol 43:401-415; Fire A. et al (1998) Nature 391:806-811; WO 99/32619; WO 99/53050; WO 00/68374; WO 00/44914; WO 00/44895; WO 00/49035; WO 00/63364). The processes and methods described in the above references are expressly referred to. Efficient gene suppression can also be demonstrated in the case of transient expression or following transient transformation, for example as the result of a biolistic transformation (Schweizer P et al. (2000) Plant J 2000 24: 895-903). dsRNAi methods are based on the phenomenon that the simultaneous introduction of complementary strand and counterstrand of a gene transcript brings about a highly efficient suppression of the expression of the gene in question. The phenotype which is brought about greatly resembles one of a corresponding knockout mutant (Waterhouse P M et al. (1998) Proc Natl Acad Sci USA 95:13959-64).

The dsRNAi method has proved to be especially efficient and advantageous for reducing expression. As described in WO 99/32619, inter alia, dsRNAi approaches are markedly superior to traditional antisense approaches.

The invention therefore also relates to double-stranded RNA molecules (dsRNA molecules) which, when introduced into a plant (or a cell, tissue, organs, in particular leaf epidermis derived therefrom), bring about the reduction of a BI-1.

In the double-stranded RNA molecule for reducing the expression of a BI-1 protein,
a) one of the two RNA strands is essentially identical with at least a part of a BI-1 nucleic acid sequence, and
b) the respective other RNA strand is essentially identical with at least a part of the complementary strand of a BI-1 nucleic acid sequence.

In a furthermore preferred embodiment, the double-stranded RNA molecule for reducing the expression of a BI-1 protein comprises:
a) a sense RNA strand comprising at least one ribo-nucleotide sequence which is essentially identical with at least a part of the sense RNA transcript of a nucleic acid sequence coding for a BI-1 protein, and
b) an antisense RNA strand which is essentially—preferably fully—complementary to the RNA sense strand of a).

With regard to the double-stranded RNA molecules, BI-1 nucleic acid sequence preferably refers to a sequence of SEQ ID NO: 1, 3, 5, 7, 9, 11, 13, 15, 17, 19, 21, 23, 25, 27, 29, 31, 33 or 38 or a functional equivalent of the same.

“Essentially identical”-means that the dsRNA sequence can also comprise insertions, deletions and individual point mutations in comparison with the BI-1 target sequence while still efficiently bringing about a reduction of the expression. Preferably, the homology between the sense strand of an inhibitory dsRNA and a partial segment of a BI-1 nucleic acid sequence (or between the antisense strand and the complementary strand of a BI-1 nucleic acid sequence) as defined above amounts to at least 50% or 75%, preferably to at least 80%, very especially preferably to at least 90%, most preferably to 100%.

The length of the partial segment amounts to at least 10 bases, preferably to at least 25 bases, especially preferably to at least 50 bases, very especially preferably to at least 100 bases, most preferably to at least 200 bases or at least 300 bases. As an alternative, an “essentially identical” dsRNA can also be defined as a nucleic acid sequence which is capable of hybridizing with a part of a BI-1 gene transcript (for example in 400 mM NaCl, 40 mM PIPES pH 6.4, 1 mM EDTA at 50° C. or 70° C. for 12 to 16 hours).

“Essentially complementary” means that the antisense RNA strand may also comprise insertions, deletions and individual point mutations in comparison with the complement of the sense RNA strand. The homology between the antisense RNA strand and the complement of the sense RNA strand preferably amounts to at least 80%, preferably to at least 90%, very especially preferably to at least 95%, most preferably to 100%.

“Part of the sense RNA transcript” of a nucleic acid sequence coding for a BI-1 protein or a functional equivalent thereof refers to fragments of an RNA or mRNA transcribed from a nucleic acid sequence coding for a BI-1 protein or a functional equivalent thereof, preferably from a BI-1 gene. In this context, the fragments preferably have a sequence length of at least 2.0 bases, preferably at least 50 bases, especially preferably at least 100 bases, very especially preferably at least 200 bases, most preferably at least 500 bases. Also comprised is the complete transcribed RNA or mRNA.

Also comprised is the use of the dsRNA molecules according to the invention in the methods according to the invention for generating a pathogen resistance in plants.

The dsRNA can consist of one or more strands of polymerized ribonucleotides. Furthermore, modifications of both the sugar-phosphate backbone and of the nucleotides may be present. For example, the phosphodiester bonds of the natural RNA can be modified in such a way that they comprise at least one nitrogen or sulfur hetero atom. Bases can be modified in such a way that the activity of, for example, adenosine deaminase is limited. These and further modifications are described further below in the methods for stabilizing antisense RNA.

To achieve the same purpose, it is naturally also possible to introduce, into the cell or the organism, a plurality of individual dsRNA molecules, each of which comprises one of the above-defined ribonucleotide sequence segments.

The dsRNA can be prepared enzymatically or fully or partially by chemical synthesis.

The double-stranded dsRNA structure can be formed starting from two complementary, separate RNA strands or—preferably—starting from a single autocomplementary RNA strand.

The double-stranded structure can be formed starting from a single autocomplementary strand or starting from two complementary strands. In the case of a single auto-complementary strand, sense and antisense sequences can be linked by a linking sequence (“linker”) and form for example a hairpin structure. The linking sequence can preferably be an intron, which is spliced out after the dsRNA has been synthesized. The nucleic acid sequence coding for a dsRNA can comprise further elements such as, for example, transcription termination signals or polyadenylation signals. If the two strands of the dsRNA are to be combined in a cell or plant, this can be effected in different ways:

The nucleic acid sequence coding for a dsRNA can comprise further elements, such as, for example, transcription termination signals or polyadenylation signals.

If the two strands of the dsRNA are to be combined in a cell or plant, this can be effected in different ways:
a) transformation of the cell or plant with a vector which comprises both expression cassettes,
b) cotransformation of the cell or plant with two vectors, one comprising the expression cassettes with the sense strand, the other comprising the expression cassettes with the antisense strand.
c) Hybridization of two plants, each of which has been transformed with one vector, one comprising the expression cassettes with the sense strand, the other comprising the expression cassettes with the antisense strand.

The formation of the RNA duplex can be initiated either externally of the cell or within the same. As in WO 99/53050, the dsRNA can also comprise a hairpin structure by linking sense and antisense strands by a linker (for example an intron). The autocomplementary dsRNA structures are preferred since they only require the expression of one construct and always comprise the complementary strands in an equimolar ratio.

The expression cassettes encoding the antisense or sense strand of a dsRNA or the autocomplementary strand of the dsRNA are preferably inserted, under the control of an epidermis-specific promoter as detailed herein, into a vector and, using the methods described hereinbelow, stably inserted into the genome of a plant in order to ensure permanent expression of the dsRNA in the epidermis, using selection markers for example.

The dsRNA can be introduced using a quantity which allows at least one copy per cell. Greater quantities (for example at least 5, 10, 100, 500 or 1000 copies per cell) may bring about a more effective reduction, if appropriate.

As already described, 100% sequence identity between dsRNA and a BI-1 gene transcript or the gene transcript of a functionally equivalent gene is not necessarily required in order to bring about an effective reduction of the expression of BI-1. Accordingly, there is the advantage that the method is tolerant with regard to sequence deviations as may exist as the consequence of genetic mutations, polymorphisms or evolutionary divergences. Thus, for example, it is possible to use the dsRNA generated on the basis of the BI-1 sequence of one organism to suppress the expression of BI-1 in another organism. The high sequence homology between the BI-1 sequences from rice, maize and barley allows the conclusion that this protein is conserved to a high degree within plants, so that the expression of a dsRNA derived from one of the disclosed BI-1 sequences as shown in SEQ ID NO: 1, 3 or 5 appears to have an advantageous effect in other plant species as well.

Furthermore, owing to the high homology between the individual BI-1 proteins and their functional equivalents, it is possible using a single dsRNA generated from a certain BI-1 sequence of an organism to suppress the expression of further homologous BI-1 proteins and/or their functional equivalents of the same organism or else the expression of BI-1 proteins in other related species. For this purpose, the dsRNA preferably comprises sequence regions of BI-1 gene transcripts which correspond to conserved regions. Said conserved regions can easily be found by comparing sequences.

The dsRNA can be synthesized either in vivo or in vitro. To this end, a DNA sequence coding for a dsRNA can be brought into an expression cassette under the control of at least one genetic control element (such as, for example, promoter, enhancer, silencer, splice donor or splice acceptor or polyadenylation signal), an epidermis-specific expression of the dsRNA being desired. Suitable advantageous constructions are described hereinbelow. Polyadenylation is not required, nor do elements for initiating translation have to be present.

A dsRNA can be synthesized chemically or enzymatically. Cellular RNA polymerases or bacteriophage RNA polymerases (such as, for example, T3, T7 or SP6 RNA polymerase) can be used for this purpose. Suitable methods for expression of RNA in vitro are described (WO 97/32016; U.S. Pat. No. 5,593,874; U.S. Pat. No. 5,698,425, U.S. Pat. No. 5,712,135, U.S. Pat. No. 5,789,214, U.S. Pat. No. 5,804,693). A dsRNA which has been synthesized in vitro chemically or enzymatically can be isolated completely or to some degree from the reaction mixture, for example by extraction, precipitation, electrophoresis, chromatography or combinations of these methods, before being introduced into a cell, tissue or organism. The dsRNA can be introduced directly into the cell or else be applied extracellularly (for example into the interstitial space).

However, it is preferred to transform the plant stably with an expression construct which brings about the expression of the dsRNA in the epidermis. Suitable methods are described hereinbelow.

b) Introduction of a BI-1 Antisense Nucleic Acid Molecule

Methods for suppressing a specific protein by preventing its mRNA from accumulating by means of antisense technology have been described in many instances, including in the case of plants (Sheehy et al. (1988) Proc Natl Acad Sci USA 85: 8805-8809; U.S. Pat. No. 4,801,340; Mol J N et al. (1990) FEBS Lett 268(2):427-430). The antisense nucleic acid molecule hybridizes, or binds, with the cellular mRNA and/or genomic DNA encoding the BI-1 target protein to be suppressed. This suppresses the transcription and/or translation of the target protein. Hybridization can originate conventionally by the formation of a stable duplex or—in the case of genomic DNA—by the antisense nucleic acid molecule binding to the duplex of the genomic DNA by specific interaction in the major groove of the DNA helix. The introduction is effected in such a way that the amount or function of BI-1 is reduced specifically in the epidermis, for example by transient transformation of the epidermis or stable transformation under the expressional control of a suitable construct with an epidermis-specific promoter.

An antisense nucleic acid sequence suitable for reducing a BI-1 protein can be deduced using the nucleic acid sequence encoding this protein, for example the nucleic acid sequence as shown in SEQ ID NO: 1, 3, 5, 7, 9, 11, 13, 15, 17, 19, 21, 23, 25, 27, 29, 31, 33 or 38, or coding for a functional equivalent thereof, following Watson and Crick's base-pairing rules. The antisense nucleic acid sequence can be complementary to all of the transcribed mRNA of said protein, be limited to the coding region, or else only be composed of an oligonucleotide, which is partially complementary to the coding or noncoding sequence of the mRNA. Thus, for example, the oligonucleotide can be complementary to the region comprising the translation start for said protein. Antisense nucleic acid sequences can be, for example, 5, 10, 15, 20, 25, 30, 35, 40, 45 or 50 nucleotides in length, but may also be longer and comprise at least 100, 200, 500, 1000, 2000 or 5000 nucleotides. Antisense nucleic acid sequences can be expressed recombinantly or synthesized chemically or enzymatically using methods known to the skilled worker. In the case of chemical synthesis, natural or modified nucleotides may be used. Modified nucleotides can impart an increased biochemical stability to the antisense nucleic acid sequence and lead to an increased physical stability of the duplex formed of antisense nucleic acid sequence and sense target sequence. The following can be used: for example phosphorothioate derivatives and acridine-substituted nucleotides such as 5-fluorouracil, 5-bromouracil, 5-chlorouracil, 5-iodouracil, hypoxanthin, xanthin, 4-acetylcytosine, 5-(carboxyhydroxylmethyl)uracil, 5-carboxy-methylaminomethyl-2-thiouridine, 5-carboxymethyl-aminomethyluracil, dihydrouracil, β-D-galactosylqueosine, inosine, N6-isopentenyladenine, 1-methylguanine, 1-methyl-inosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N-6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid, 5-methyl-2-thiouracil, 3-(3-amino-3-N-2-carboxypropyl)uracil and 2,6-diaminopurine.

In a further preferred embodiment, the expression of a BI-1 protein can be inhibited by nucleotide sequences which are complementary to the regulatory region of a BI-1 gene (for example a BI-1 promoter and/or enhancer) and which form triple-helical structures with that DNA double helix so that the transcription of the BI-1 gene is reduced. Such methods have been described (Helene C (1991) Anticancer Drug Res 6(6):569-84; Helene C et al. (1992) Ann NY Acad Sci 660:27-36; Maher L J (1992) Bioassays 14(12):807-815).

In a further embodiment, the antisense nucleic acid molecule can be an α-anomeric nucleic acid. Such α-anomeric nucleic acid molecules form specific double-stranded hybrids with complementary RNA in which—as opposed to the conventional β-nucleic acids—the two strands run parallel to one another (Gautier C et al. (1987) Nucleic Acids Res 15:6625-6641). The antisense nucleic acid molecule can furthermore also comprise 2′-O-methylribonucleotides (Inoue et al. (1987) Nucleic Acids Res 15:6131-6148) or chimeric RNA/DNA analogs (Inoue et al. (1987) FEBS Lett 215:327-330).

c) Introduction of a BI-1 Antisense Nucleic Acid Molecule in Combination with a Ribozyme

The above-described antisense strategy can be combined advantageously with a ribozyme method. Catalytic RNA molecules or ribozymes can be adapted to suit any target RNA and cleave the phosphodiester backbone at specific positions, functionally deactivating the target RNA (Tanner N K (1999) FEMS Microbiol Rev 23(3):257-275). The ribozyme itself is not modified thereby, but is capable of cleaving further target RNA molecules analogously, thereby assuming the qualities of an enzyme. The incorporation of ribozyme sequences into antisense RNAs confers this enzyme-like RNA-cleaving quality to precisely these antisense RNAs, thus increasing their efficacy in inactivating the target RNA. The generation and the use of such ribozyme antisense RNA molecules is described, for example, in Haselhoff et al. (1988) Nature 334: 585-591.

In this manner, ribozymes (for example “hammerhead” ribozymes; Haselhoff and Gerlach (1988) Nature 334:585-591) can be used catalytically to cleave the mRNA of an enzyme to be suppressed, for example BI-1, and to prevent translation. The ribozyme technique can increase the efficacy of an antisense strategy. Methods of expressing ribozymes for reducing specific proteins are described in EP 0 291 533, EP 0 321 201, EP 0 360 257. The expression of ribozyme in plant cells has also been described (Steinecke P et al. (1992) EMBO J 11(4):1525-1530; de Feyter R et al. (1996) Mol Gen Genet. 250(3):329-338). Suitable target sequences and ribozymes can be determined as described for example by “Steinecke P, Ribozymes, Methods in Cell Biology 50, Galbraith et al. eds, Academic Press, Inc. (1995), pp. 449 460” by calculating the secondary structure of ribozyme RNA and target RNA as well as by their interaction (Bayley C C et al. (1992) Plant Mol. Biol. 18(2):353-361; Lloyd A M and Davis R W et al. (1994) Mol Gen Genet. 242(6):653-657). For example, derivatives of the Tetrahymena L-19 IVS RNA with regions which are complementary to the mRNA of the BI-1 protein to be suppressed can be constructed (see also U.S. Pat. No. 4,987,071 and U.S. Pat. No. 5,116,742). As an alternative, such ribozymes can also be identified from a library of diverse ribozymes via a selection process (Bartel D and Szostak J W (1993) Science 261:1411-1418). Expression takes place, for example, under the control of an epidermis-specific promoter.

d) Introduction of a BI-1 Sense Nucleic Acid Molecule for Inducing Cosuppression

The epidermis-specific expression of a BI-1 nucleic acid molecule in sense orientation can lead to cosuppression, in the epidermis cells, of the corresponding homologous endogenous gene. The expression of sense RNA with homology with an endogenous gene can reduce or switch off the expression of the former, similarly to what has been described for antisense approaches (Jorgensen et al. (1996) Plant Mol Biol 31(5):957-973; Goring et al. (1991) Proc Natl Acad Sci USA 88:1770-1774; Smith et al. (1990) Mol Gen Genet 224:447-481; Napoli et al. (1990) Plant Cell 2:279-289; Van der Krol et al. (1990) Plant Cell 2:291-99). In this context, the homologous gene to be reduced can be represented either fully or only in part by the construct introduced. The possibility of translation is not required. The application of this technique to plants is described, for example, by Napoli et al. (1990) The Plant Cell 2: 279-289 and in U.S. Pat. No. 5,034,323.

The cosuppression is preferably realized by using a sequence essentially identical with at least a part of the nucleic acid sequence coding for a BI-1 protein or a functional equivalent thereof, for example the nucleic acid sequence as shown in SEQ ID NO: 1, 3, 5, 7, 9, 11, 13, 15, 17, 19, 21, 23, 25, 27, 29, 31, 33 or 38 or the nucleic acid sequence coding for a functional equivalent thereof.

e) Introduction of Nucleic Acid Molecules Coding for a Dominant-Negative BI-1 Protein

The function or activity of a BI-1 protein can also be reduced efficiently in epidermis cells by expressing, in an epidermis-specific manner, a dominant-negative variant of this BI-1 protein. Methods of reducing the function or activity of a protein by coexpressing its dominant-negative form are known to the skilled worker (Lagna G and Hemmati-Brivanlou A (1998) Current Topics in Developmental Biology 36:75-98; Perlmutter R M and Alberola-Ila J (1996) Current Opinion in Immunology 8(2):285-90; Sheppard D (1994) American Journal of Respiratory Cell & Molecular Biology. 11(1):1-6; Herskowitz I (1987) Nature 329(6136):219-22).

The amino acid which is preferably to be mutated in BI-1 homologs from other species can be determined for example by computer-aided comparison (“alignment”). These mutations for achieving a dominant-negative BI-1 variant are preferably carried out at the level of the nucleic acid sequence coding for BI-1 proteins. A corresponding mutation can be brought about for example by PCR-mediated in-vitro mutagenesis using suitable oligonucleotide primers, by which the desired mutation is introduced. This is done using methods known to the skilled worker; for example, the “LA PCR in vitro Mutagenesis Kit” (Takara Shuzo, Kyoto) may be used for this purpose. A method of generating a dominant-negative variant of a maize RacB protein is also described in WO 00/15815 (Example 4, p. 69).

Such a mutant can then be expressed for example under the control of an epidermis-specific promoter.

f) Introduction of DNA- or Protein-Binding Factors Against BI-1 Genes, BI-1 RNAs or BI-1 Proteins

BI-1 gene expression in the epidermis may also be reduced using specific DNA-binding factors, for example factors of the zinc finger transcription factor type. These factors attach to the genomic sequence of the endogenous target gene, preferably in the regulatory regions, and bring about repression of the endogenous gene. The use of such a method makes the reduction of the expression of an endogenous BI-1 gene possible without it being necessary to recombinantly manipulate its sequence. Suitable methods for the preparation of suitable factors have been described (Dreier B et al. (2001) J Biol Chem 276(31):29466-78; Dreier B et al. (2000) J Mol Biol 303(4):489-502; Beerli R R et al. (2000) Proc Natl Acad Sci USA 97 (4):1495-1500; Beerli R R et al. (2000) J Biol Chem 275(42):32617-32627; Segal D J and Barbas C F 3rd. (2000) Curr Opin Chem Biol 4(1):34-39; Kang J S and Kim J S (2000) J Biol Chem 275(12):8742-8748; Beerli R R et al. (1998) Proc Natl Acad Sci USA 95(25):14628-14633; Kim J S et al. (1997) Proc Natl Acad Sci USA 94(8):3616-3620; Klug A (1999) J Mol Biol 293(2):215-218; Tsai S Y et al. (1998) Adv Drug Deliv Rev 30(1-3):23-31; Mapp A K et al. (2000) Proc Natl Acad Sci USA 97(8):3930-3935; Sharrocks A D et al. (1997) Int J Biochem Cell Biol 29(12):1371-1387; Zhang L et al. (2000) J Biol Chem 275(43):33850-33860).

These factors can be selected using any desired portion of a BI-1 gene. This segment is preferably located in the promoter region. For gene suppression, however, it may also be in the region of the coding exons or introns. The segments in question can be obtained by the skilled worker from Genbank by database search or, starting from a BI-1 cDNA whose gene is not present in Genbank, by screening a genomic library for corresponding genomic clones. The skilled worker is familiar with the methods required therefor, for example, these factors can be expressed under the control of an epidermis-specific promoter or other factors which mediate epidermis-specific expression.

Furthermore, it is possible to introduce, into a cell, factors which inhibit the BI-1 target protein itself. The protein-binding factors can be, for example, aptamers (Famulok M and Mayer G (1999) Curr Top Microbiol Immunol 243:123-36) or antibodies or antibody fragments or single-chain antibodies. Methods for obtaining these factors have been described and are known to the skilled worker. For example, a cytoplasmic scFv antibody was employed to modulate the activity of the phytochrome A protein in genetically modified tobacco plants (Owen M et al. (1992) Biotechnology (N Y) 10(7):790-794; Franken E et al. (1997) Curr Opin Biotechnol 8(4):411-416; Whitelam (1996) Trend Plant Sci 1:286-272).

Gene expression may also be suppressed by tailor-made low-molecular-weight synthetic compounds, for example of the polyamide type (Dervan P B and Bürli R W (1999) Current Opinion in Chemical Biology 3:688-693; Gottesfeld J M et al. (2000) Gene Expr 9(1-2):77-91). These oligomers are composed of the units 3-(dimethylamino)propylamine, N-methyl-3-hydroxypyrrole, N-methylimidazole and N-methylpyrrole and can be adapted to any piece of double-stranded DNA in such a way that they bind into the major groove in a sequence-specific manner and block the expression of these gene sequences. Suitable methods have been described (see, inter alia, Bremer R E et al. (2001) Bioorg Med. Chem. 9(8):2093-103; Ansari A Z et al. (2001) Chem Biol. 8(6):583-92; Gottesfeld J M et al. (2001) J Mol. Biol. 309(3):615-29; Wurtz N R et al. (2001) Org Lett 3(8):1201-3; Wang C C et al. (2001) Bioorg Med Chem 9(3):653-7; Urbach A R and Dervan P B (2001) Proc Natl Acad Sci USA 98(8):4343-8; Chiang S Y et al. (2000) J Biol. Chem. 275(32):24246-54).

All the abovementioned factors are introduced in an epidermis-specific manner in order to ensure a reduction of the BI-1 activity only in epidermal cells, for example by means of expression under the control of an epidermis-specific promoter as they are mentioned for example hereinabove.

g) Introduction of Viral Nucleic Acid Molecules and Corresponding Expression Constructs which Cause the Degradation of BI-1 RNA

BI-1 expression in the epidermis can also be brought about efficiently by inducing the specific degradation of BI-1 RNA in epidermal cells with the aid of a viral expression system (amplicon) (Angell, S M et al. (1999) Plant J. 20(3):357-362). These systems—also termed “VIGS” (viral induced gene silencing)—introduce, into the plant, nucleic acid molecules with homology to the transcripts to be suppressed, with the aid of viral vectors. Then, transcription is switched off, probably mediated by plant defense mechanisms against viruses. Suitable techniques and methods have been described (Ratcliff F et al. (2001) Plant J 25(2):237-45; Fagard M and Vaucheret H (2000) Plant Mol Biol 43(2-3):285-93; Anandalakshmi R et al. (1998) Proc Natl Acad Sci USA 95(22):13079-84; Ruiz M T (1998) Plant Cell 10(6): 937-46).

h) Introduction of Constructs for Inducing Homologous Recombination at Endogenous BI-1 Genes, for Example for Generating Knockout Mutants

An example of what is used for generating a homologously recombinant organism with reduced BI-1 activity in the epidermal cells is a nucleic acid construct comprising at least a part of an endogenous BI-1 gene which is modified by a deletion, addition or substitution of at least one nucleotide in such a way that its functionality is reduced or fully destroyed. The modification may also relate to the regulatory elements (for example the promoter) of the gene, so that the coding sequence remains unmodified, but expression (transcription and/or translation) does not take place and is reduced.

In the case of conventional homologous recombination, the modified region is flanked at its 5′ and 3′ ends by further nucleic acid sequences which must be sufficient in length for making recombination possible. They are, as a rule, in the range of several hundred bases to several kilobases in length (Thomas K R and Capecchi M R (1987) Cell 51:503; Strepp et al. (1998) Proc Natl Acad Sci USA 95(8): 4368-4373). For homologous recombination, the host organism—for example a plant—is transformed with the recombination construct using the methods described hereinbelow, and clones which have successfully undergone recombination are selected, for example using a resistance to antibiotics or herbicides.

Homologous recombination is a relatively rare event in higher eukaryotes, especially in plants. Random integrations into the host genome predominate. One possibility of eliminating the randomly integrated sequences and thus increasing the number of cell clones with a correct homologous recombination is the use of a sequence-specific recombination system as described in U.S. Pat. No. 6,110,736, by which unspecifically integrated sequences can be deleted again, which simplifies the selection of events which have integrated successfully via homologous recombination. A large number of sequence-specific recombination systems can be used, examples being the Cre/lox system of bacteriophage P1, the FLP/FRT system of yeast, the Gin recombinase of phage Mu, the Pin recombinase from E. coli, and the R/RS system of the pSR1 plasmid. The bacteriophage P1 Cre/10× and the yeast FLP/FRT system are preferred. The FLP/FRT and cre/lox recombinase system has already been applied to plant systems (Odell et al. (1990) Mol Gen Genet 223: 369-378). Epidermis-specific recombination can be ensured for example by the expression of the systems and enzymes which mediate recombination taking place under the control of an epidermis-specific promoter.

i) Introduction of Mutations into Endogenous BI-1 Genes for Generating a Loss of Function (for Example Generation of Stop Codons, Reading-Frame Shifts and the Like)

Further suitable methods for reducing the BI-1 activity are the introduction of nonsense mutations into endogenous BI-1 genes, for example by introducing RNA/DNA oligonucleotides into the epidermal cells (Zhu et al. (2000) Nat Biotechnol 18(5):555-558) and the generation of knockout mutants with the aid of, for example, T-DNA mutagenesis (Koncz et al. (1992) Plant Mol Biol 20(5):963-976), ENU (N-ethyl-N-nitrosourea) mutagenesis or homologous recombination (Hohn B and Puchta (1999) H Proc Natl Acad Sci USA 96:8321-8323). Point mutations can also be generated by means of DNA-RNA hybrids also known under the name “chimeraplasty” (Cole-Strauss et al. (1999) Nucl Acids Res 27(5):1323-1330; Kmiec (1999) Gene therapy American Scientist 87(3):240-247).

The methods of dsRNAi, cosuppression by means of sense RNA and “VIGS” (“virus induced gene silencing”) are also termed “post-transcriptional gene silencing” (PTGS). PTGS methods, like the reduction of the BI-1 function or activity with dominant-negative BI-1 variants, are especially advantageous because the demands regarding the homology between the endogenous gene to be suppressed and the sense or dsRNA nucleic acid sequence expressed recombinantly (or between the endogenous gene and its dominant-negative variant) are lower than, for example, in the case of a traditional antisense approach. Such criteria with regard to homology are mentioned in the description of the dsRNAi method and can generally be applied to PTGS methods or dominant-negative approaches. Thus, using the BI-1 nucleic acid sequences, it is presumably also possible efficiently to suppress the expression of homologous BI-1 proteins in other species without the isolation and structure elucidation of the BI-1 homologs occurring therein being required. Considerably less labor is therefore required. Similarly, the use of dominant-negative variants of a BI-1 protein can presumably also efficiently reduce or suppress the function/activity of its homolog in other plant species.

All of the substances and compounds which directly or indirectly bring about a reduction in protein quantity, RNA quantity, gene activity or protein activity of a BI-1 protein shall subsequently be combined in the term “anti-BI-1” compounds. The term “anti-BI-1” compound explicitly includes the nucleic acid sequences, peptides, proteins or other factors employed in the above-described methods.

For the purposes of the invention, “introduction” comprises all of the methods which are capable of directly or indirectly introducing an “anti-BI-1” compound into the epidermis or a substantial number of the epidermal cells, compartments or tissues thereof, or of generating such a compound there. Direct and indirect methods are comprised. The introduction can lead to a transient presence of an “anti-BI-1” compound (for example of a dsRNA) or else to its stable presence.

Owing to the different nature of the above-described approaches, the “anti-BI-1” compound can exert its function directly (for example by insertion into an endogenous BI-1 gene). However, its function can also be exerted indirectly following transcription into an RNA (for example in the case of antisense approaches) or following transcription and translation into a protein (for example in the case of binding factors). The invention comprises both directly and indirectly acting “anti-BI-1” compounds.

The term “introducing” comprises for example methods such as transfection, transduction or transformation.

The term “anti-BI-1” compounds therefore also comprises recombinant expression constructs, for example, which bring about an expression (i.e. transcription and, if appropriate, translation), for example of a BI-1 dsRNA or a BI-1 “antisense” RNA in an epidermis-specific manner.

In said expression constructs, a nucleic acid molecule whose expression (transcription and, if appropriate, translation) generates an “anti-BI-1” compound is preferably operably linked to at least one genetic control element (for example a promoter) which ensures expression in an organism, preferably in plants, and preferably in an epidermis-specific manner. If the expression construct is to be introduced directly into the plant and the “anti-BI-1” compound (for example the BI-1 dsRNA) is to be generated therein in planta, plant-specific genetic control elements (for example promoters) are preferred, where, as the result of what has been said above, the epidermis-specific activity of the promoter is mandatory in most embodiments for an epidermis-specific reduction of BI-1, as described above. However, the “anti-BI-1” compound may also be generated in other organisms or in vitro and then be introduced into the plant. Preferred in this context are all of the prokaryotic or eukaryotic genetic control elements (for example promoters) which permit the expression in the organism chosen in each case for the preparation.

Functional linkage is to be understood as meaning, for example, the sequential arrangement of a promoter with the nucleic acid sequence to be expressed (for example an “anti-BI-1” compound) and, if appropriate, further regulatory elements such as, for example, a terminator in such a way that each of the regulatory elements can fulfill its function when the nucleic acid sequence is expressed recombinantly, depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules (localization in cis and trans respectively). Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs.

Functional linkage, and an expression cassette, can be generated by means of customary recombination and cloning techniques as are described, for example, in Maniatis T, Fritsch E F and Sambrook J (1989) Molecular Cloning: A Laboratory Manual, Cold Spring Harbor Laboratory, Cold Spring Harbor (NY), in Silhavy T J, Berman M L and Enquist L W (1984) Experiments with Gene Fusions, Cold Spring Harbor Laboratory, Cold Spring Harbor (NY), in Ausubel F M et al. (1987) Current Protocols in Molecular Biology, Greene Publishing Assoc. and Wiley Interscience and in Gelvin et al. (1990) In: Plant Molecular Biology Manual. However, further sequences which, for example, act as a linker with specific cleavage sites for restriction enzymes, or as a signal peptide, may also be positioned between the two sequences. The insertion of sequences may also lead to the expression of fusion proteins. Preferably, the expression cassette, consisting of a linkage of promoter and nucleic acid sequence to be expressed, can exist in a vector-integrated form and be inserted into a plant genome, for example by transformation. The control elements preferably mediate an epidermis-specific expression.

The abovementioned methods (a) to (i) can also be employed for the reduction of the activity or function, in particular the expression, of the other proteins mentioned herein, in particular for the reduction of MLO, RacB and NaOx.

The barley BI1 protein (hvBI1) is predominantly expressed in the mesophyll (Example 6) and is upregulated as the result of infection with Blumeria (syn. Erysiphe) graminis f. sp. hordei (Example 2). The recombinant mesophyll-specific overexpression in mlo-resistant barley leads not only to resistance to in particular necrotrophic and hemibiotrophic pathogens, but also to a plant which is resistant to Blumeria (syn. Erysiphe) graminis f. sp. hordei and which exhibits no necrotic lesions (“mlo lesions”; negative side effect of the mlo resistance). Utilizing this effect, the negative side effects of the mlo-mediated resistance (yield loss of approximately 5%, Jorgensen J H (1992) Euphytica 63: 141-152; hypersusceptibility to necrotrophic fungi, Jarosch B et al. (1999) Mol Plant Microbe Interact 12:508-514; Kumar. J et al. (2001) Phytopathology 91:127-133) can be suppressed without adversely affecting the mlo resistance itself.

Furthermore, it can be demonstrated in a surprising manner that an overexpression of BI1 results in resistance to stress factors like agents which trigger necroses (isolated for example from necrotrophic harmful fungi; Example 2).

Accordingly, the method according to the invention offers an efficient biotechnological strategy for resistance to the formation of necroses as the result of endogenous, abiotic and biotic stress, for example mlo lesions, ozone damage, necrotrophic and hemibiotrophic harmful organisms.

BI1 proteins appear to be crucial regulators of the race-unspecific fungal resistance in plants. This makes possible their broad use in biotechnological strategies for increasing the pathogen resistance in plants, in particular fungal resistance. The method according to the invention can be applied to all plant species, in principle. BI1 proteins have been identified in a large number of plants, both monocots and dicots (see above).

For the purposes of the present invention, “approximately”, when referring to numbers or sizes, means a range of numbers or sizes around the stated value of the number or size. In general, the term approximately means a range of in each case 20% above and below of the value stated.

The term “plant” as used herein refers to all genera and species of higher and lower plants of the Plant Kingdom. The term includes the mature plants, seed, shoots and seedlings and their derived parts, propagation material, plant organs, tissue, protoplasts, callus and other cultures, for example cell cultures, and any other types of plant-cell associations to give functional or structural units. The term mature plants refers to plants at any desired developmental stage beyond that of the seedling. Seedling refers to a young immature plant at an early developmental stage.

“Plant” comprises all annual and perennial monocotyledonous and dicotyledonous plants and includes by way of example but not by limitation those of the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Antirrhinum, Hemerocallis, Nemesis, Pelargonium, Panicum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea and Populus.

The term “plant” preferably comprises monocotyledonous crop plants such as, for example, cereal species such as wheat, barley, millet, rye, triticale, maize, rice, sorghum or oats and also sugar cane.

The term furthermore comprises dicotyledonous crop plants such as, for example,
Brassicaceae such as oilseed rape, turnip rape, cress, Arabidopsis, cabbage species,
Leguminosae such as soybean, alfalfa, pea, beans or peanut,
Solanaceae such as potato, tobacco, tomato, egg plant or capsicum,
Asteraceae such as sunflower, Tagetes, lettuce or Calendula,
Cucurbitaceae such as melon, pumpkin/squash or zucchini,
   and also linseed (flax), cotton, hemp, clover, spinach, red pepper, carrot, beet, radish, sugar beet, sweet potato, cucumber, chicory, cauliflower, broccoli, asparagus, onion, garlic, celery/celeriac, strawberry, raspberry, blackberry, pineapple, avocado and the various tree, bush, nut and vine species. Tree species preferably comprise plum, cherry, peach, nectarine, apricot, banana, papaya, mango, apple, pear, quince.

Preferred within the scope of the invention are those plants which are employed as foodstuffs or feeding stuffs, very especially preferably monocotyledonous genera and agriculturally important species such as wheat, oats, millet, barley, rye, maize, rice, buckwheat, sorghum, triticale, spelt, linseed or sugar cane.

For the purposes of the present invention, the term “stress factor” comprises biotic stress factors (such as in particular the pathogens detailed hereinbelow) and abiotic stress factors. Abiotic stress factors which may be mentioned by way of example, but not by limitation, are: chemical stress (for example caused by agrochemicals and/or environmental chemicals), UV radiation, heat, cold, drought, increased humidity.

“Stress resistance” means the reduction or alleviation of symptoms of a plant as a result of stress. The symptoms can be manifold, but preferably comprise those which directly or indirectly have an adverse effect on the quality of the plant, the quantity of its yield, its suitability for use as feeding stuff or foodstuff, or else make sowing, planting, harvesting or processing of the crop more difficult.

“Pathogen resistance” denotes the reduction or alleviation of disease symptoms of a plant following infection by at least one pathogen. The symptoms can be manifold, but preferably comprise those which directly or indirectly have an adverse effect on the quality of the plant, the quantity of the yield, its suitability for use as feeding stuff or foodstuff, or else which make sowing, planting, harvesting or processing of the crop more difficult.

“Conferring”, “generating” or “increasing” a pathogen resistance and “the existence of” a pathogen resistance means that the defense mechanisms of a specific plant species or variety are increasingly resistant to one or more stress factors or pathogens due to the use of the method according to the invention in comparison with the wild type of the plant (“starting plant”), to which the method according to the invention has not been applied, under otherwise identical conditions (such as, for example, climatic conditions, growing conditions, type of stress or pathogen species and the like). The increased resistance manifests itself preferably in a reduced manifestation of the stress or disease symptoms, disease symptoms comprising—in addition to the abovementioned adverse effects—for example also the penetration efficiency of a pathogen into the plant or plant cells or the proliferation efficiency in or on the same. In this context, the stress or disease symptoms are preferably reduced by at least 10% or at least 20%, especially preferably by at least 40% or 60%, very especially preferably by at least 70% or 80% and most preferably by at least 90% or 95%.

“Selection” with regard to plants in which—as opposed or as compared to the starting plant—resistance to at least one stress factor or pathogen exists or is increased means all those methods which a are suitable for recognizing an existing or increased resistance to stress or pathogens. For example, these may be symptoms of pathogen infection (for example the development of necroses in the case of fungal infection), but may also comprise the above-described symptoms which relate to the quality of the plant, the quantity of the yield, the suitability for use as feeding stuff or foodstuff and the like.

“Pathogen” within the scope of the invention means by way of example but not by limitation viruses or viroids, bacteria, fungi, animal pests such as, for example, insects or nematodes. Especially preferred are fungi, especially necrotrophic or hemibiotrophic fungi. However, it can be assumed that the mesophyll-specific expression of a BI1 protein also brings about resistance to other pathogens since an overall resistance to stress factors is generated.

The following pathogens may be mentioned by way of example but not by limitation:

1. Fungal Pathogens or Fungus-Like Pathogens:

Fungal pathogens or fungus-like pathogens (e.g. Chromista) are preferably from the group comprising Plasmodiophoramycota, Oomycota, Ascomycota, Chytridiomycetes, Zygomycetes, Basidiomycota and Deuteromycetes (Fungi imperfecti). The pathogens mentioned in Tables 1 to 4 and the diseases with which they are associated may be mentioned by way of example but not by limitation. The following English and German terms can be used interchangeably:
Ährenfäule—ear rot/head blight
Stengelfäule—stalk rot
Wurzelfäule—root rot
Rost—rust
Falscher Mehltau—downy mildew

Further translations can be found for example at http://www.bba.de/english/database/psmengl/pilz.htm.

TABLE 1
Diseases caused by biotrophic phytopathogenic
fungi

| Disease | Pathogen |
|---|---|
| Brown rust | Puccinia recondita |
| Yellow rust | P. striiformis |
| Powdery mildew | Erysiphe graminis/Blumeria |
| | graminis |
| Rust (common maize) | Puccinia sorghi |
| Rust (southern corn) | Puccinia polysora |
| Tobacco frogeye disease | Cercospora nicotianae |
| Rust (tropical maize) | Physopella pallescens, P. zeae = Angiopsora |
| | zeae |

TABLE 2
Diseases caused by necrotrophic and/or hemibiotrophic fungi and Oomycetes

| Disease | Pathogen |
|---|---|
| Glume blotch | Septoria (Stagonospora) nodorum |
| Leaf blotch | Septoria tritici |
| Ear fusarioses | Fusarium spp. |
| Eyespot | Pseudocercosporella herpotrichoides |
| Smut | Ustilago spp. |
| Potato blight | Phytophthora infestans |
| Bunt | Tilletia caries |
| Blackleg | Gaeumannomyces graminis |
| Anthrocnose leaf blight | Colletotrichum graminicola |
| Anthracnose stalk rot | (teleomorph: Glomerella graminicola |
| | Politis); Glomerella tucumanensis |
| | (anamorph: Glomerella falcatum |
| | Went) |
| Aspergillus ear and | Aspergillus flavus |
| kernel rot | |
| Banded leaf and sheath spot | Rhizoctonia solani Kuhn = Rhizoctonia |
| | microsclerotia J. Matz |
| | (telomorph: Thanatephorus |
| | cucumeris) |
| Black bundle disease | Acremonium strictum W. Gams = Cephalosporium |
| | acremonium Auct. non |
| | Corda |
| Black kernel rot | Lasiodiplodia theobromae = Botryodiplodia |
| | theobromae |
| Borde blanco | Marasmiellus sp. |
| Brown spot (black spot, | Physoderma maydis |
| stalk rot) | |
| Cephalosporium kernel rot | Acremonium strictum = Cephalosporium |
| | acremonium |
| Charcoal rot | Macrophomina phaseolina |
| Corticium ear rot | Thanatephorus cucumeris = Corticium |
| | sasakii |
| Curvularia leaf spot | Curvularia clavata, C. eragrostidis, = C. maculans |
| | (teleomorph: Cochliobolus |
| | eragrostidis), Curvularia |
| | inaequalis, C. intermedia |
| | (teleomorph: Cochliobolus |
| | intermedius), Curvularia lunata |
| | (teleomorph: Cochliobolus lunatus), |
| | Curvularia pallescens (teleomorph: |
| | Cochliobolus pallescens), |
| | Curvularia senegalensis, C. tuberculata |
| | (teleomorph: |
| | Cochliobolus tuberculatus) |
| Didymella leaf spot | Didymella exitalis |
| Diplodia ear and stalk rot | Diplodia frumenti (teleomorph: |
| | Botryosphaeria festucae) |
| Diplodia ear and stalk rot, | Diplodia maydis = Stenocarpella |
| seed rot and seedling blight | maydis |
| Diplodia leaf spot or streak | Stenocarpella macrospora = Diplodialeaf |
| | macrospora |
| Brown stripe downy | Sclerophthora rayssiae var. zeae |
| mildew | |
| Crazy top downy mildew | Sclerophthora macrospora = Sclerospora |
| | macrospora |
| Green ear downy mildew | Sclerospora graminicola |
| (graminicola downy mildew) | |
| Dry ear rot (cob, | Nigrospora oryzae |
| kernel and stalk rot) | (teleomorph: Khuskia oryzae) |
| Ear rots/head blights | Alternaria alternata = A. tenuis, |
| (minor) | Aspergillus glaucus, A. niger, |
| | Aspergillus spp., Botrytis cinerea |
| | (teleomorph: Botryotinia |
| | fuckeliana), Cunninghamella sp., |
| | Curvularia pallescens, |
| | Doratomyces stemonitis = Cephalotrichum |
| | stemonitis, |
| | Fusarium culmorum, |
| | Gonatobotrys simplex, |
| | Pithomyces maydicus, |
| | Rhizopus microsporusTiegh., |
| | R. stolonifer = R. nigricans, |
| | Scopulariopsis brumptii |
| Ergot(horse's tooth) | Claviceps gigantea |
| | (anamorph: Sphacelia sp.) |
| Eyespot | Aureobasidium zeae = Kabatiella |
| | zeae |
| Fusarium ear and stalk rot | Fusarium subglutinans = F. moniliforme |
| | var. subglutinans |
| Fusarium kernel, root and | Fusarium moniliforme |
| stalk rot, seed rot and | (teleomorph: Gibberella fujikuroi) |
| seedling blight | |
| Fusarium stalk rot, | Fusarium avenaceum |
| seedling root rot | (teleomorph: Gibberella avenacea) |
| Gibberella ear and stalk rot | Gibberella zeae |
| | (anamorph: Fusarium graminearum) |
| Gray ear rot/head blight | Botryosphaeria zeae = Physalospora |
| | zeae (anamorph: Macrophoma zeae) |
| Gray leaf spot | Cercospora sorghi = C. sorghi var. |
| (Cercospora leaf spot) | maydis, C. zeae-maydis |
| Helminthosporium root rot | Exserohilum pedicellatum = Helminthosporium |
| | pedicellatum |
| | (teleomorph: Setosphaeria |
| | pedicellata) |
| Hormodendrum ear rot/head | Cladosporium cladosporioides = Hormodendrum |
| blight (Cladosporium rot) | cladosporioides, C. herbarum |
| | (teleomorph: |
| | Mycosphaerella tassiana) |
| Leaf spots, minor | Alternaria alternata, |
| | Ascochyta maydis, A. tritici, |
| | A. zeicola, Bipolaris victoriae = Helminthosporium |
| | victoriae |
| | (teleomorph: Cochliobolus |
| | victoriae), C. sativus (anamorph: |
| | Bipolaris sorokiniana = H. sorokinianum = H. sativum), |
| | Epicoccum nigrum, |
| | Exserohilum prolatum = Drechslera |
| | prolata (teleomorph: Setosphaeria |
| | prolata) |
| | Graphium penicillioides, |
| | Leptosphaeria maydis, Leptothyrium |
| | zeae, Ophiosphaerella herpotricha, |
| | (anamorph: Scolecosporiella sp.), |
| | Paraphaeosphaeria michotii, Phoma |
| | sp., Septoria zeae, S. zeicola, |
| | S. zeina |
| Northern corn leaf blight | Setosphaeria turcica (anamorph: |
| (white blast, crown stalk | Exserohilum turcicum = Helminthosporium |
| rot, stripe) | turcicum) |
| Northern corn leaf spot | Cochliobolus carbonum (anamorph: |
| Helminthosporium ear rot | Bipolaris zeicola = Helminthosporium |
| (race 1) | carbonum) |
| Blue eye, blue mold | Penicillium spp., P. chrysogenum, |
| | P. expansum, P. oxalicum |
| Phaeocytostroma stalk and | Phaeocytostroma ambiguum, = Phaeocytosporella |
| root rot | zeae |
| Phaeosphaeria leaf spot | Phaeosphaeria maydis = Sphaerulina |
| | maydis |
| Physalospora ear rot/head | Botryosphaeria festucae = Physalospora |
| blight (Botryosphaeria ear | zeicola (anamorph: |
| rot/head blight) | Diplodia frumenti) |
| Purple leaf sheath | Hemiparasitic bacteria and fungi |
| Pyrenochaeta stalk and root | Phoma terrestris = Pyrenochaeta |
| rot | terrestris |
| Pythium root rot | Pythium spp., P. arrhenomanes, |
| | P. graminicola |
| Pythium stalk rot | Pythium aphanidermatum = P. butleri L. |
| Red kernel disease (ear | Epicoccum nigrum |
| mold, leaf and seed rot) | |
| Sclerotial rot | Rhizoctonia zeae (teleomorph: |
| | Waitea circinata) |
| Rhizoctonia root and stalk | Rhizoctonia solani, Rhizoctonia |
| rot | zeae |
| Root rots (minor) | Alternaria alternata, Cercospora |
| | sorghi, Dictochaeta fertilis, |
| | Fusarium acuminatum (teleomorph: |
| | Gibberella acuminata), F. equiseti |
| | (teleomorph: G. intricans), F. oxysporum, |
| | F. pallidoroseum, F. poae, |
| | F. roseum, G. cyanogena, |
| | (anamorph: F. sulphureum), |
| | Microdochium bolleyi, Mucor sp., |
| | Periconia circinata, Phytophthora |
| | cactorum, P. drechsleri, P. nicotianae |
| | var. parasitica, |
| | Rhizopus arrhizus |
| Rostratum leaf spot | Setosphaeria rostrata, (anamorph: |
| (Helminthosporium leaf | Exserohilum rostratum = Helminthosporium |
| disease, ear and stalk rot) | rostratum) |
| Java downy mildew | Peronosclerospora maydis = Sclerospora |
| | maydis |
| Philippine downy mildew | Peronosclerospora philippinensis = Sclerospora |
| | philippinensis |
| Sorghum downy mildew | Peronosclerospora sorghi = Sclerospora |
| | sorghi |
| Spontaneum downy mildew | Peronosclerospora spontanea = Sclerospora |
| | spontanea |
| Sugar cane downy mildew | Peronosclerospora sacchari = Sclerospora |
| | sacchari |
| Southern blight | Sclerotium rolfsii Sacc. |
| | (teleomorph: Athelia rolfsii) |
| Seed rot-seedling blight | Bipolaris sorokiniana, B. zeicola = Helminthosporium |
| | carbonum, Diplodia |
| | maydis, Exserohilum pedicillatum, |
| | Exserohilum turcicum = Helminthosporium |
| | turcicum, Fusarium |
| | avenaceum, F. culmorum, F. moniliforme, |
| | Gibberella zeae |
| | (anamorph: F. graminearum), |
| | Macrophomina phaseolina, |
| | Penicillium spp., Phomopsis sp., |
| | Pythium spp., Rhizoctonia solani, |
| | R. zeae, Sclerotium rolfsii, |
| | Spicaria sp. |
| Selenophoma leaf spot | Selenophoma sp. |
| Sheath rot | Gaeumannomyces graminis |
| Shuck rot | Myrothecium gramineum |
| Silage mold | Monascus purpureus, M ruber |
| Common smut | Ustilago zeae = U. maydis |
| False smut | Ustilaginoidea virens |
| Head smut | Sphacelotheca reiliana = Sporisorium |
| | holcisorghi |
| Southern corn leaf blight | Cochliobolus heterostrophus |
| and stalk rot | (anamorph: Bipolaris maydis = Helminthosporium |
| | maydis) |
| Southern leaf spot | Stenocarpella macrospora = Diplodia |
| | macrospora |
| Stalk rots (minor) | Cercospora sorghi, Fusarium |
| | episphaeria, F. merismoides, F. oxysporum |
| | Schlechtend, F. poae, F. roseum, |
| | F. solani (teleomorph: |
| | Nectria haematococca), F. tricinctum, |
| | Mariannaea elegans, |
| | Mucor sp., Rhopographus zeae, |
| | Spicaria sp. |
| Storage rots | Aspergillus spp., Penicillium spp. |
| | and other fungi |
| Tar spot | Phyllachora maydis |
| Trichoderma ear rot and root | Trichoderma viride = T. lignorum |
| rot | teleomorph: Hypocrea sp. |
| White ear rot, root and | Stenocarpella maydis = Diplodia |
| stalk rot | zeae |
| Yellow leaf blight | Ascochyta ischaemi, Phyllosticta |
| | maydis (teleomorph: Mycosphaerella |
| | zeae-maydis) |
| Zonate leaf spot | Gloeocercospora sorghi |

TABLE 4
Diseases caused by fungi and Oomycetes whose
classification with regard to biotrophic,
hemibiotrophic or necrotrophic behavior is unclear

| Disease | Pathogen |
|---|---|
| Hyalothyridium leaf spot | Hyalothyridium maydis |
| Late wilt | Cephalosporium maydis |

The following are especially preferred:
Plasmodiophoromycota such as Plasmodiophora brassicae (clubroot), Spongospora subterranea, Polymyxa graminis,
Oomycota such as Bremia lactucae (downy mildew on lettuce), Peronospora (downy mildew) in the case of antirrhinum (P. antirrhini), onion (P. destructor), spinach (P. effusa), soybean (P. manchurica), tobacco (blue mold; P. tabacina), alfalfa and clover (P. trifolium), Pseudoperonospora humuli (downy mildew on hops), Plasmopara (downy mildew in the case of grapes) (P. viticola) and sunflower (P. halstedii), Sclerophtohra macrospora (downy mildew in the case of cereals and grasses), Pythium (e.g. blackleg on Beta beet by P. debaryanum), Phytophthora infestans (potato blight, late blight of tomato etc.), Albugo spec.

Ascomycota such as Microdochium nivale (snow mold of rye and wheat), Fusarium graminearum, Fusarium culmorum (culm rot, inter alia of wheat), Fusarium oxysporum (Fusarium wilt of tomato), Blumeria graminis (powdery mildew of barley (f.sp. hordei) and wheat (f.sp. tritici)), Erysiphe pisi (powdery mildew of pea), Nectria galligena (nectria canker of fruit trees), Unicnula necator (powdery mildew of grapevine), Pseudopeziza tracheiphila (red fire disease of grapevine), Claviceps purpurea (ergot on, for example, rye and grasses), Gaeumannomyces greminis (take-all on wheat, rye and other grasses), Magnaporthe grisea, Pyrenophora graminea (leaf stripe of barley), Pyrenophora teres (net blotch of barley), Pyrenophora tritici-repentis (leaf blight of wheat), Venturia inaequalis (apple scab), Sclerotinia sclerotiorum (stalk break, stem rot), Pseudopeziza medicaginis (leaf spot of alfalfa, white and red clover).
Basidiomycetes such as Typhula incarnata (typhula blight on barley, rye, wheat), Ustilago maydis (blister smut on maize), Ustilago nuda (loose smut on barley), Ustilago tritici (loose smut on wheat, spelt), Ustilago avenae (loose smut on oats), Rhizoctonia solani (rhizoctonia root rot of potato), Sphacelotheca spp. (head smut of sorghum), Melampsora lini (rust of flax), Puccinia graminis (stem rust of wheat, barley, rye, oats), Puccinia recondita (leaf rust of wheat), Puccinia dispersa (brown rust of rye), Puccinia hordei (leaf rust of barley), Puccinia coronata (crown rust of oats), Puccinia striiformis (yellow rust of wheat, barley, rye and a large number of grasses), Uromyces appendiculatus (brown rust of bean), Sclerotium rolfsii (root and stalk rots of many plants).

Deuteromycetes (Fungi imperfecti) such as Septoria (Stagonospora) nodorum (glume blotch) of wheat (Septoria tritici), Pseudocercosporella herpotrichoides (eyespot of wheat, barley, rye), Rynchosporium secalis (leaf spot on rye and barley), Alternaria solani (early blight of potato, tomato), Phoma betae (blackleg on Beta beet), Cercospora beticola (leaf spot on Beta beet), Alternaria brassicae (black spot on oilseed rape, cabbage and other crucifers), Verticillium dahliae (verticillium wilt), Colletotrichum lindemuthianum (bean anthracnose), Phoma lingam (blackleg of cabbage and oilseed rape), Botrytis cinerea (gray mold of grapevine, strawberry, tomato, hops and the like).

Most preferred are Phytophthora infestans (potato blight, brown rot in tomato and the like), Microdochium nivale (previously Fusarium nivale; snow mold of rye and wheat), Fusarium graminearum, Fusarium culmorum, Fusarium avenaceum and Fusarium poae (ear rot/head blight of wheat), Fusarium oxysporum (Fusarium wilt of tomato), Magnaporthe grisea (rice blast disease), Sclerotinia sclerotium (stalk break, stem rot), Septoria (Stagonospora) nodorum and Septoria tritici (glume blotch of wheat), Alternaria brassicae (black spot on oilseed rape, cabbage and other crucifers), Phoma lingam (blackleg of cabbage and oilseed rape).

2. Bacterial Pathogens:

The pathogens and the diseases associated with them which are mentioned in Table 5 may be mentioned by way of example but not by limitation.

TABLE 5
Bacterial diseases

| Disease | Pathogen |
|---|---|
| Bacterial leaf blight and | Pseudomonas avenae subsp. avenae |
| stalk rot | |
| Bacterial leaf spot | Xanthomonas campestris pv. holcicola |
| Bacterial stalk rot | Enterobacter dissolvens = Erwinia |
| | dissolvens |
| Bacterial stalk and top rot | Erwinia carotovora subsp. |
| | carotovora, Erwinia chrysanthemi pv. |
| | zeae |
| Bacterial stripe | Pseudomonas andropogonis |
| Chocolate spot | Pseudomonas syringae pv. |
| | coronafaciens |
| Goss's bacterial wilt and | Clavibacter michiganensis subsp. |
| blight (leaf freckles and | nebraskensis = Corynebacterium |
| wilt) | michiganense pv. andnebraskense |
| Holcus spot | Pseudomonas syringae pv. syringae |
| Purple leaf sheath | Hemiparasitic bacteria |
| Seed rot-seedling blight | Bacillus subtilis |
| Stewart's disease | Pantoea stewartii = Erwinia |
| (bacterial wilt) | stewartii |
| Corn stunt | Spiroplasma kunkelii |
| (achapparramiento, maize | |
| stunt, Mesa Central or Rio | |
| Grande maize stunt) | |

The following pathogenic bacteria are very especially preferred: Corynebacterium sepedonicum (bacterial ring rot of potato), Erwinia carotovora (blackleg of potato), Erwinia amylovora (fire blight of pear, apple, quince), Streptomyces scabies (potato scab), Pseudomonas syringae pv. tabaci (wildfire of tobacco), Pseudomonas syringae pv. phaseolicola (grease spot of dwarf bean), Pseudomonas syringae pv. tomato (bacterial speck of tomato), Xanthomonas campestris pv. malvacearum (bacterial blight of cotton) and Xanthomonas campestris pv. oryzae (bacterial leaf blight of rice and other grasses).

3. Viral Pathogens:

“Viral pathogens” includes all plant viruses such as, for example, tobacco or cucumber mosaic virus, ringspot virus, necrosis virus, maize dwarf mosaic virus and the like.

The pathogens and diseases associated with them which are mentioned in Table 6 may be mentioned by way of example, but not by limitation.

TABLE 6
Viral diseases

| Disease | Pathogen |
|---|---|
| American wheat striate | American wheat striate mosaic virus |
| (wheat striate mosaic) | (AWSMV) |
| Barley stripe mosaic | Barley stripe mosaic virus (BSMV) |
| Barley yellow dwarf | Barley yellow dwarf virus (BYDV) |
| Brome mosaic | Brome mosaic virus (BMV) |
| Cereal chlorotic mottle | Cereal chlorotic mottle virus (CCMV) |
| Corn chlorotic vein | Corn chlorotic vein banding virus |
| banding (Brazilian maize | (CCVBV) |
| mosaic) | |
| Corn lethal necrosis | Virus complex of Maize chlorotic |
| | mottle virus (MCMV) and Maize dwarf |
| | mosaic virus (MDMV) A or B or Wheat |
| | streak mosaic virus(WSMV) |
| Cucumber mosaic | Cucumber mosaic virus (CMV) |
| Cynodon chlorotic streak | Cynodon chlorotic streak virus (CCSV) |
| Johnsongrass mosaic | Johnsongrass mosaic virus (JGMV) |
| Maize bushy stunt | Mycoplasma-like organism (MLO) |
| | associated |
| Maize chlorotic dwarf | Maize chlorotic dwarf virus (MCDV) |
| Maize chlorotic mottle | Maize chlorotic mottle virus (MCMV) |
| Maize dwarf mosaic | Maize dwarf mosaic virus (MDMV) |
| | strains A, D, E and F |
| Maize leaf fleck | Maize leaf fleck virus (MLFV) |
| Maize line | Maize line virus (MLV) |
| Maize mosaic (corn leaf | Maize mosaic virus (MMV) |
| stripe, enanismo rayado) | |
| Maize mottle and | Maize mottle and chlorotic stunt virus |
| chlorotic stunt | |
| Maize pellucid ringspot | Maize pellucid ringspot virus (MPRV) |
| Maize raya gruesa | Maize raya gruesa virus (MRGV) |
| Maize rayado fino (fine | Maize rayado fino virus (MRFV) |
| striping disease) | |
| Maize red leaf and red | Mollicute |
| stripe | |
| Maize red stripe | Maize red stripe virus (MRSV) |
| Maize ring mottle | Maize ring mottle virus (MRMV) |
| Maize rio IV | Maize rio cuarto virus (MRCV) |
| Maize rough dwarf | Maize rough dwarf virus (MRDV) (Cereal |
| (nanismo ruvido) | tillering disease virus) |
| Maize sterile stunt | Maize sterile stunt virus (strains |
| | of barley yellow striate virus) |
| Maize streak | Maize streak virus (MSV) |
| Maize stripe (maize | Maize stripe virus |
| chlorotic stripe, maize | |
| hoja blanca) | |
| Maize stunting | Maize stunting virus |
| Maize tassel abortion | Maize tassel abortion virus (MTAV) |
| Maize vein enation | Maize vein enation virus (MVEV) |
| Maize wallaby ear | Maize wallaby ear virus (MWEV) |
| Maize white leaf | Maize white leaf virus |
| Maize white line mosaic | Maize white line mosaic virus (MWLMV) |
| Millet red leaf | Millet red leaf virus (MRLV) |
| Northern cereal mosaic | Northern cereal mosaic virus (NCMV) |
| Oat pseudorosette | Oat pseudorosette virus |
| (zakuklivanie) | |
| Oat sterile dwarf | Oat sterile dwarf virus (OSDV) |
| Rice black-streaked | Rice black-streaked dwarf virus |
| dwarf | (RBSDV) |
| Rice stripe | Rice stripe virus (RSV) |
| Sorghum mosaic | Sorghum mosaic virus (SrMV) (also: |
| | sugarcane mosaic virus (SCMV) strains |
| | H, I and M) |
| Sugarcane Fiji disease | Sugarcane Fiji disease virus (FDV) |
| Sugarcane mosaic | Sugarcane mosaic virus (SCMV) strains |
| | A, B, D, E, SC, BC, Sabi and MB |
| | (formerly MDMV-B) |
| Wheat spot mosaic | Wheat spot mosaic virus (WSMV) |

4. Animal Pests
4.1 Insect Pathogens:

The following may be mentioned by way of example, but not by limitation: insects such as, for example, beetles, caterpillars, aphids or mites. Preferred insects are those of the genera Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc. Especially preferred are coleopteran and lepidopteran insects such as, for example, the European corn borer (ECB), Diabrotica barberi, Diabrotica undecimpunctata, Diabrotica virgifera, Agrotis ipsilon, Crymodes devastator, Feltia ducens, Agrotis gladiaria, Melanotus spp., Aeolus mellillus, Aeolus mancus, Horistonotus uhlerii, Sphenophorus maidis, Sphenophorus zeae, Sphenophorus parvulus, Sphenophorus callosus, Phyllogphaga spp., Anuraphis maidiradicis, Delia platura, Colaspis brunnea, Stenolophus lecontei and Clivinia impressifrons.

Other examples are: lema (Oulema melanopus), frit fly (Oscinella frit), wireworms (Agrotis lineatus) and aphids (such as, for example, the oat grain aphid Rhopalosiphum padi, the blackberry aphid Sitobion avenae).

4.2 Nematodes:

The pathogens and the diseases associated with them mentioned in Table 7 may be mentioned by way of example, but not by limitation.

TABLE 7
Parasitic nematodes

| Damage | Pathogenic nematode |
|---|---|
| Awl | Dolichodorus spp., D. heterocephalus |
| Bulb and stem nematode, | Ditylenchus dipsaci |
| stem eelworm of rye | |
| Burrowing | Radopholus similis |
| Oat cyst nematode | Heterodera avenae, H. zeae, |
| | Punctodera chalcoensis |
| Dagger | Xiphinema spp., X. americanum, |
| | X. mediterraneum |
| False root-knot | Nacobbus dorsalis |
| Lance, Columbia | Hoplolaimus columbus |
| Lance | Hoplolaimus spp., H. galeatus |
| Lesion | Pratylenchus spp., P. brachyurus, |
| | P. crenatus, P. hexincisus, |
| | P. neglectus, P. penetrans, |
| | P. scribneri, P. thornei, P. zeae |
| Needle | Longidorus spp., L. breviannulatus |
| Ring | Criconemella spp., C. ornata |
| Root-knot nematode | Meloidogyne spp., M. chitwoodi, |
| | M. incognita, M. javanica |
| Spiral | Helicotylenchus spp. |
| Sting | Belonolaimus spp., B. longicaudatus |
| Stubby-root | Paratrichodorus spp., P. christiei, |
| | P. minor, Quinisulcius acutus, |
| | Trichodorus spp. |
| Stunt | Tylenchorhynchus dubius |

Very especially preferred are Globodera rostochiensis and G. pallida (cyst eelworm on potato, tomato and other Solanaceae), Heterodera schachtii (beet cyst eelworm on sugar and fodder beet, oilseed rape, cabbage and the like), Heterodera avenae (oat cyst nematode on oats and other cereal species), Ditylenchus dipsaci (stem or bulb eelworm, stem eelworm of rye, oats, maize, clover, tobacco, beet), Anguina tritici (grain nematode, cockle disease of wheat (spelt, rye), Meloidogyne hapla (root-knot nematode of carrot, cucumber, lettuce, tomato, potato, sugar beet, lucerne).

Examples of preferred fungal or viral pathogens for the individual varieties are:

1. Barley:

Fungal, bacterial and viral pathogens: Puccinia graminis f.sp. hordei, Blumeria (Erysiphe) graminis f.sp. hordei, barley yellow dwarf virus (BYDV),

Pathogenic insects/nematodes: Ostrinia nubilalis (European corn borer); Agrotis ipsilon; Schizaphis graminum; Blissus leucopterus leucopterus; Acrosternum hilare; Euschistus servus; Deliaplatura; Mayetiola destructor; Petrobia latens.

2. Soybean:

Fungal, bacterial or viral pathogens: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotrichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffussa, Fusarium semitectum, Phialophora gregata, soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines, Fusarium solani.

Pathogenic insects/nematodes: Pseudoplusia includens; Anticarsia gemmatalis; Plathypena scabra; Ostrinia nubilalis; Agrotis ipsilon; Spodoptera exigua; Heliothis virescens; Helicoverpa zea; Epilachna varivestis; Myzus persicae; Empoasca fabae; Acrosternum hilare; Melanoplus femurrubrum; Melanoplus differentialis; Hylemya platura; Sericothrips variabilis; Thrips tabaci; Tetranychus turkestani; Tetranychus urticae.

3. Canola:

Fungal, bacterial or viral pathogens: Albugo candida, Alternaria brassicae, Leptosphaeria maculans, Rhizoctonia solani, Sclerotinia sclerotiorum, Mycosphaerella brassicola, Pythium ultimum, Peronospora parasitica, Fusarium roseum, Alternaria alternata.

4. Alfalfa:

Fungal, bacterial or viral pathogens: Clavibater michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium, Xanthomonas campestris p.v. alfalfae, Aphanomyces euteiches, Stemphylium herbarum, Stemphylium alfalfae.

5. Wheat:

Fungal, bacterial or viral pathogens: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria (Stagonospora) nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, soil borne wheat mosaic virus, wheat streak mosaic virus, wheat spindle streak virus, American wheat striate virus, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, high plains virus, European wheat striate virus, Puccinia graminis f.sp. tritici (wheat stem rust), Blumeria (Erysiphe) graminis f.sp. tritici (wheat powdery mildew).

Pathogenic insects/nematodes: Pseudaletia unipunctata; Spodoptera, frugiperda; Elasmopalpus lignosellus; Agrotis orthogonia; Elasmopalpus Zignosellus; Oulema melanopus; Hypera punctata; Diabrotica undecimpunctata howardi; Russian wheat aphid; Schizaphis graminum; Macrosiphum avenae; Melanoplus femurrubrum; Melanoplus differentialis; Melanoplus sanguinipes; Mayetiola destructor; Sitodiplosis mosellana; Meromyza americana; Hylemya coarctata; Frankliniella fusca; Cephus cinctus; Aceria tulipae;

6. Sunflower:

Fungal, bacterial or viral pathogens: Plasmophora halstedii, Sclerotinia sclerotiorum, aster yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum p.v. Carotovora, Cephalosporium acremonium, Phytophthora cryptogea, Albugo tragopogonis.

Pathogenic insects/nematodes: Suleima helianthana; Homoeosoma electellum; zygogramma exclamationis; Bothyrus gibbosus; Neolasioptera murtfeldtiana.

7. Maize:

Fungal, bacterial or viral pathogens: Fusarium moniliforme var. subglutinans, Erwinia stewartii, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis 0, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganese subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi p.v. Zea, Erwinia corotovora, Cornstunt spiroplasma, Diplodia macrospora, Sclerophthora macrospora, Peronosclerospora sorghi, Peronosclerospora philippinesis, Peronosclerospora maydis, Peronosclerospora sacchari, Spacelotheca reiliana, Physopella zeae, Cephalosporium maydis, Caphalosporium acremonium, Maize Chlorotic Mottle Virus, High Plains Virus, Maize Mosaic Virus, Maize Rayado Fino Virus, Maize Streak Virus (MSV, Maisstrichel-Virus), Maize Stripe Virus, Maize Rough Dwarf Virus.

Pathogenic insects/nematodes: Ostrinia nubilalis; Agrotis ipsilon; Helicoverpa zea; Spodoptera frugiperda; Diatraea grandiosella; Elasmopalpus lignosellus; Diatraea saccharalis; Diabrotica virgifera; Diabrotica longicornis barberi; Diabrotica undecimpunctata howardi; Melanotus spp.; Cyclocephala borealis; Cyclocephala immaculata; Popillia japonica; Chaetocnema pulicaria; Sphenophorus maidis; Rhopalosiphum maidis; Anuraphis maidiradicis; Blissus leucopterus leucopterus; Melanoplus femurrubrum; Melanoplus sanguinipes; Hylemva platura; Agromyza parvicornis; Anaphothrips obscrurus; Solenopsis milesta; Tetranychus urticae.

8. Sorghum:

Fungal, bacterial or viral pathogens: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternate, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola.

Pathogenic insects/nematodes: Chilo partellus; Spodoptera frugiperda; Helicoverpa zea; Elasmopalpus lignosellus; Feltia subterranea; Phyllophaga crinita; Eleodes, Conoderus and Aeolus spp.; Oulema melanopus; Chaetocnema pulicaria; Sphenophorus maidis; Rhopalosiphum maidis; Sipha flava; Blissus leucopterus leucopterus; Contarinia sorghicola; Tetranychus cinnabarinus; Tetranychus urticae.

9. Cotton:

Pathogenic insects/nematodes: Heliothis virescens; Helicoverpa zea; Spodoptera exigua; Pectinophora gossypiella; Anthonomus grandis grandis; Aphis gossypii; Pseudatomoscelis seriatus; Trialeurodes abutilonea; Lygus lineolaris; Melanoplus femurrubrum; Melanoplus differentialis; Thrips tabaci (onion thrips); Franklinkiella fusca; Tetranychus cinnabarinus; Tetranychus urticae.

10. Rice:

Pathogenic insects/nematodes: Diatraea saccharalis; Spodoptera frugiperda; Helicoverpa zea; Colaspis brunnea; Lissorhoptrus oryzophilus; Sitophilus oryzae; Nephotettix nigropictus; Blissus leucopterus; Acrosternum hilare.

11. Oilseed Rape:

Pathogenic insects/nematodes: Brevicoryne brassicae; Phyilotreta cruciferae; Mamestra conjgurata; Plutella xylostella; Delia ssp.

For the purposes of the invention, “BI1 protein” is understood as meaning polypeptides which have at least one sequence with at least 50%, preferably at least 80%, especially preferably at least 90%, very especially preferably 100% homology with a BI1 consensus motif selected from the group consisting of

a) H(L/I)KXVY
b) AXGA(Y/F)XH
c) NIGG
d) P(V/P) (Y/F)E(E/Q) (R/Q)KR
e) (E/Q)G(A/S)S(V/I)GPL
f) DP(S/G) (L/I) (I/L)
g) V(G/A)T(A/S) (L/I)AF(A/G)CF(S/T)
h) YL(Y/F)LGG, preferably EYLYLGG
i) L(L/V)SS(G/W)L(S/T) (I/M)L(L/M)W
j) DTGX(I/V) (I/V)E.

Especially preferred in this context is the BI consensus motif f) YL(Y/F)LGG, very especially preferably (EYLYLGG). This motif is characteristic of plant BI1 proteins.

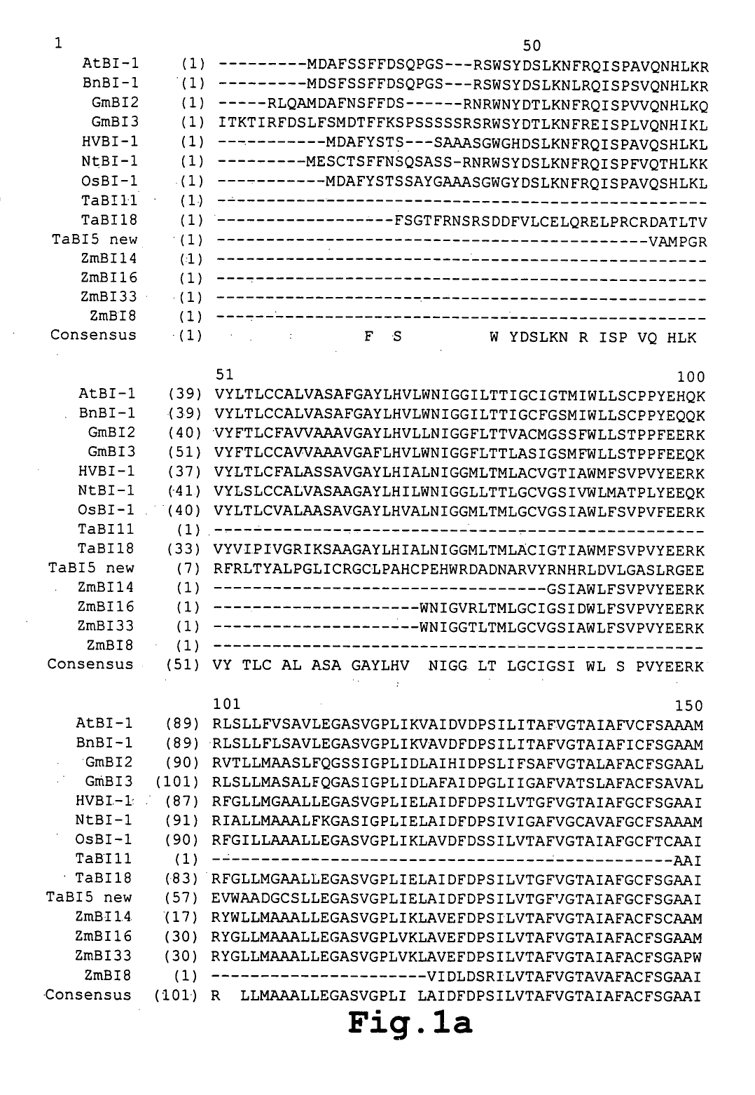

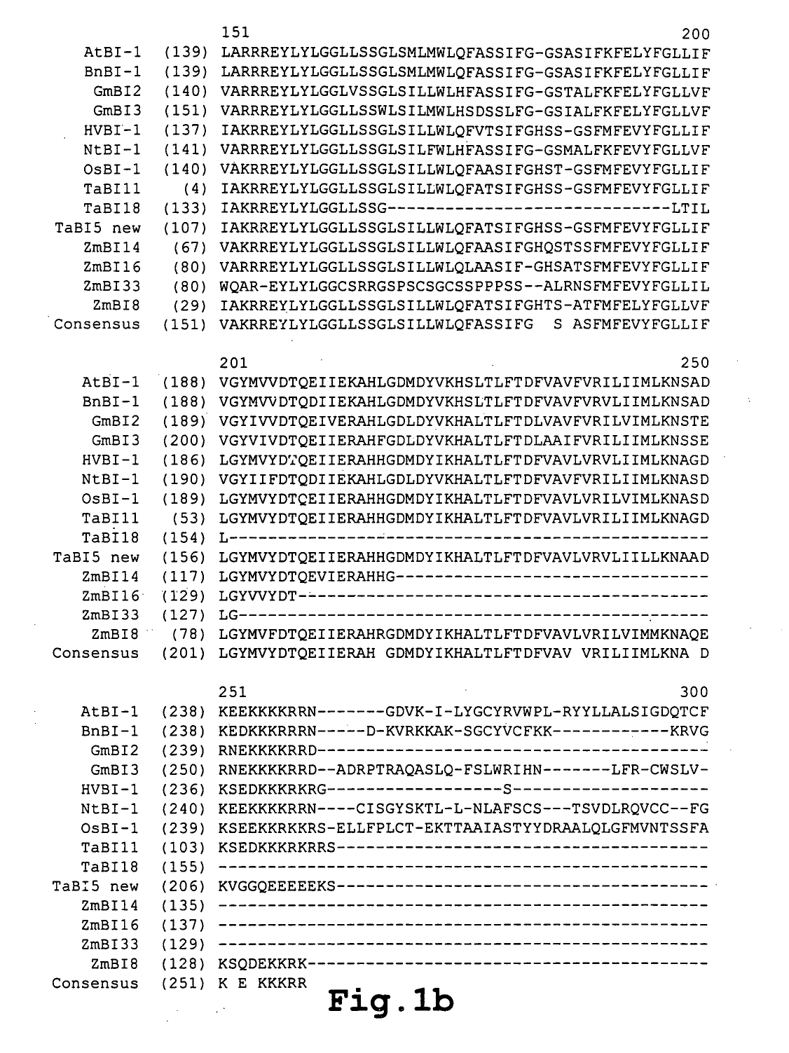

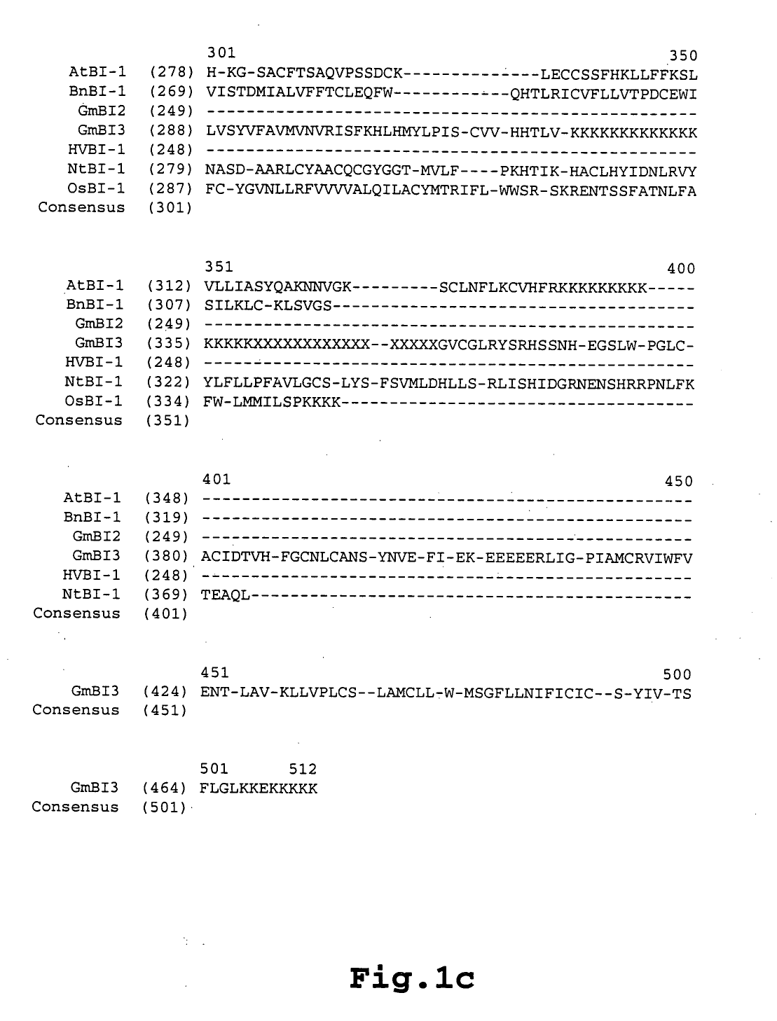

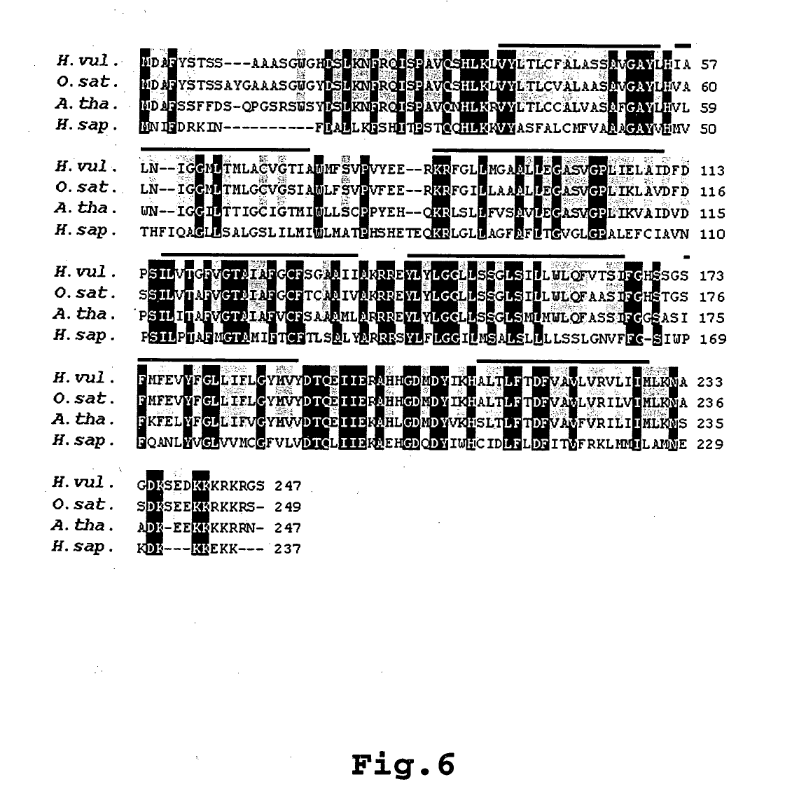

It is especially preferred that sequences with homology to at least 2 or 3 of these motifs (a to g) occur in a BI1 protein, very especially preferably at least 4 or 5, most preferably all motifs a to j. Further BI1-typical sequence motifs can be derived by the skilled worker without difficulty from the sequence alignment of BI1 proteins as shown in FIG. 1 or 6.

Particularly preferable are BI proteins which are encoded by a polypeptide comprising at least one sequence selected from the group consisting of:
a) the sequences as shown in SEQ ID NO: 2, 4, 6, 8, 10, 12, 14, 16, 18, 20, 22, 24, 26, 28, 30, 32 and 38, and
b) sequences which have at least 50%, preferably at least 70%, especially preferably at least 90%, very especially preferably at least 95% identity with one of the sequences as shown in SEQ ID NO: 2, 4, 6, 8, 10, 12, 14, 16, 18, 20, 22, 24, 26, 28, 30, 32 and 38,
c) sequences which comprise at least one part-sequence of at least 10, preferably 20, especially preferably 50 contiguous amino acid residues of one of the sequences as shown in SEQ ID NO: 2, 4, 6, 8, 10, 12, 14, 16, 18, 20, 22, 24, 26, 28, 30, 32 and 38.

In accordance with the invention, the term BI protein comprises in particular natural or artificial mutations of the BI1 polypeptide as shown in SEQ ID NO: 2, 4, 6, 8, 10 and 38 and homologous polypeptides from other organisms, preferably plants, which continue to have essentially identical characteristics. Mutations comprise substitutions, additions, deletions, inversions or insertions of one or more amino acid residues. This means that use forms using BI1 proteins from nonplant organisms such as, for example, humans (GenBank Acc. No.: P55061), rats (GenBank Acc. No.: P55062) or Drosophila (GenBank Acc. No.: Q9VSH3) are also comprised. Motifs which are conserved between plant and nonplant BI1 proteins can be identified easily by sequence alignment (cf. Alignment in Bolduc et 1. (2003) Planta 216:377-386; FIGS. 1 and 6).

Thus, polypeptides which are also comprised by the present invention are for example those which are obtained by modification of a polypeptide as shown in SEQ ID NO: 2, 4, 6, 8, 10 and 38.

The sequences, from other plants, which are homologous to the BI1 sequences disclosed within the scope of the present invention can be identified for example by
a) database search in libraries of organisms whose genomic sequence or cDNA sequence is known in its entirety or in part, using the BI1 sequences provided as search sequence or
b) screening gene libraries or cDNA libraries using the BI1 sequences provided as probes.

Screening cDNA libraries or genomic libraries (for example using one of the nucleic acid sequences described in SEQ ID NO: 1, 3, 5, 7, 9, 11, 13, 15, 17, 19, 21, 23, 25, 27, 29, 31 and 37 or parts of these as probes) is a method, known to the skilled worker, for identifying homologs in other species. In this context, the probes derived from the nucleic acid sequences as shown in SEQ ID NO: 1, 3, 5, 7, 9, 11, 13, 15, 17, 19, 21, 23, 25, 27, 29, 31 and 37 have a length of at least 20 bp, preferably at least 50 bp, especially preferably at least 100 bp, very especially preferably at least 200 bp, most preferably at least 200 bp. A DNA strand which is complementary to the sequences described as SEQ ID NO: 1, 3, 5, 7, 9, 11, 13, 15, 17, 19, 21, 23, 25, 27, 29, 31 and 37 may also be used for screening the libraries.

Homology between two nucleic acid sequences is understood as meaning the identity of the nucleic acid sequence over in each case the entire sequence length which is calculated by comparison with the aid of the program algorithm GAP (Wisconsin Package Version 10.0, University of Wisconsin, Genetics Computer Group (GCG), Madison, USA; Altschul et al. (1997) Nucleic Acids Res. 25:3389 et seq.), setting the following parameters:

| | |
|---|---|
| Gap weight: 50 | Length weight: 3 |
| Average match: 10 | Average mismatch: 0 |

For example a sequence which has at least 80% homology with sequence SEQ ID NO: 1 at the nucleic acid level is understood as meaning a sequence which, upon comparison with the sequence SEQ ID NO: 1 by the above program algorithm with the above parameter set, has at least 80% homology.

Homology between two polypeptides is understood as meaning the identity of the amino acid sequence over in each case the entire sequence length which is calculated by comparison with the aid of the program algorithm GAP (Wisconsin Package Version 10.0, University of Wisconsin, Genetics Computer Group (GCG), Madison, USA), setting the following parameters:

| | |
|---|---|
| Gap weight: 8 | Length weight: 2 |
| Average match: 2,912 | Average mismatch: −2,003 |

For example a sequence which has at least 80% homology with sequence SEQ ID NO: 2 at the protein level is understood as meaning a sequence which, upon comparison with the sequence SEQ ID NO: 2 by the above program algorithm with the above parameter set, has at least 80% homology.

BI1 proteins also comprise those polypeptides which are encoded by nucleic acid sequences which hybridize under standard conditions with a BI1 nucleic acid sequence described by SEQ ID NO: 1, 3, 5, 7, 9, 11, 13, 15, 17, 19, 21, 23, 25, 27, 29, 31 and 37, with the nucleic acid sequence complementary thereto or with parts of the above and which have the same essential characteristics as the proteins described as SEQ ID NO: 2, 4, 6, 8, 10 and 38.

“Standard hybridization conditions” is to be understood in the broad sense and means stringent or else less stringent hybridization conditions. Such hybridization conditions are described, inter alia, by Sambrook J, Fritsch E F, Maniatis T et al., in Molecular Cloning (A Laboratory Manual), 2nd Edition, Cold Spring Harbor Laboratory Press, 1989, pages 9.31-9.57) or in Current Protocols in Molecular Biology, John Wiley & Sons, N.Y. (1989), 6.3.1-6.3.6. For example, the conditions during the wash step can be selected from the range of conditions delimited by low-stringency conditions (approximately 2×SSC at 50° C.) and high-stringency conditions (approximately 0.2×SSC at 50° C., preferably at 65° C.) (20×SSC: 0.3M sodium citrate, 3M NaCl, pH 7.0). In addition, the temperature during the wash step can be raised from low-stringency conditions at room temperature, approximately 22° C., to higher-stringency conditions at approximately 65° C. Both of the parameters, salt concentration and temperature, can be varied simultaneously, or else one of the two parameters can be kept constant while only the other is varied. Denaturants, for example formamide or SDS, may also be employed during the hybridization. In the presence of 50% formamide, hybridization is preferably effected at 42° C.

Referring to a BI protein, “ essential characteristics” means for example one or more of the following characteristics:
a) Conferring or increasing the pathogen resistance to at least one pathogen when increasing the amount of protein or function of said BI protein in at least one tissue of the plant, said tissue being other than the leaf epidermis.
b) Absence of a spontaneously induced cell death when increasing the amount of protein or the function of the said BI protein.
c) The characteristic of significantly inhibiting the BAX-induced apoptosis in the case of transient cotransfection of Bax and said BI1 protein, for example in HEK293 cells. Suitable methods are described (Bolduc N et al. (2003) Planta 216:377-386).
d) The presence of five to seven putative transmembrane domains within said BI1 protein.
e) Preferential localization in cell membranes, in particular in the nuclear membrane, the ER membrane and/or the thylakoid membrane.

In this context, the quantitative manifestation of said characteristics of a BI1 protein can deviate in both directions in comparison with a value obtained for the BI1 protein as shown in SEQ ID NO: 2, 4, 6, 8, 10 or 38.

The term “increase of the amount or function of the BI1 protein” is to be understood in the broad sense for the purposes of the present invention and may be the result of different cell-biological mechanisms.

“Amount of protein” means the amount of BI1 protein in the respective organism, tissue, cell or cell compartment.

“Increase in the amount of protein” means the quantitative increase in the amount of a BI1 protein in the respective organism, tissue, cell or cell compartment—for example by means of one of the methods described hereinbelow—in comparison with the wild type of the same genus and species to which this method has not been applied, but on the otherwise identical overall conditions (such as, for example, culture conditions, age of the plants and the like). In this context, the increase amounts to at least 10%, preferably at least 30% or at least 50%, especially preferably at least 70% or 100%, very especially preferably by at least 200% or 500%, most preferably by at least 1000%. The amount of protein can be determined by a variety of methods with which the skilled worker is familiar. The following may be mentioned by way of example, but not by way of limitation: the micro-biuret method (Goa J (1953) Scand J Clin Lab Invest 5:218-222), the Folin-Ciocalteu method (Lowry O H et al. (1951) J Biol Chem 193:265-275) or measuring the adsorption of CBB G-250 (Bradford M M (1976) Analyt Biochem 72:248-254). Furthermore, it can be quantified by immunological methods such as, for example, Western blot. The preparation of suitable BI1 antibodies and the procedure for BI1 Western blots are described (Bolduc et al. (2002) FEBS Lett 532:111-114). Indirect quantification can be effected by a Northern blot, the amount of mRNA and the resulting amount of protein showing, as a rule, good correlation. Suitable methods are described (Bolduc et al. (2003) Planta 216:377-386; Matsumura H et al. (2003) Plant J 33:425-434, inter alia).

“Function” preferably means the characteristic of a BI1 protein of reducing the spontaneously induced cell death and/or the characteristic of inhibiting the apoptosis-indicating effect of Bax. Such functions belong to the essential characteristic of a BI1 protein.

Within the context of the present invention, “increasing” the function means, for example, the quantitative increase of the inhibitory effect on the Bax-induced apoptotic cell death, which can be quantified by methods known to the skilled worker (see hereinabove). In this context, the increase amounts to at least 10%, preferably at least 30% or at least 50%, very especially preferably at least 70% or 100%, very especially preferably by at least 200% or 500%, most preferably by at least 1000%. Besides the above-described methods for increasing the amount of protein (which also always increases the function), methods for increasing the function comprise furthermore by way of example, but not by limitation, in particular the introduction of mutations into a BI1 protein.

By way of example, but not by limitation, the amount of BI1 protein can be increased by one of the following methods:
a) recombinant expression or overexpression of a BI1 protein by introducing a recombinant expression cassette comprising a nucleic acid sequence coding for a BI1 protein under the control of a tissue-specific promoter, where said promoter has essentially no activity in the leaf epidermis.
b) modification (for example substitution) of the regulatory regions (for example of the promoter region) of an endogenous BI1 gene, for example substitution of a tissue-specific promoter by means of homologous recombination, where said promoter has essentially no activity in the leaf epidermis.
c) Insertion of a nucleic acid sequence coding for a BI1 protein into the plant genome downstream of a tissue-specific promoter by means of homologous recombination, where said promoter has essentially no activity in the leaf epidermis.
d) increasing the expression of an endogenous BI1 protein by introducing a transcription factor (for example an artificial transcription factor from the class of the zinc finger proteins) which is suitable for inducing the expression of said BI1 proteins. It is preferred to introduce a recombinant expression cassette comprising a nucleic acid sequence coding for said transcription factor under the control of a tissue-specific promoter, where said promoter has essentially no activity in the leaf epidermis.

For the purposes of the invention, the term “introduction” generally comprises all those methods which are suitable for introducing, either directly or indirectly, the compound to be introduced into a plant or into a cell, compartment, tissue, organ or seed thereof, or generating it therein. Direct and indirect methods are comprised. The introduction can lead to a transient presence of said compound or else to a stable or inducible presence. Introduction comprises methods such as, for example, transfection, transduction or transformation.

In the recombinant expression cassettes which are employed within the invention, a nucleic acid molecule (for example coding for a BI1 protein) is linked functionally to at least one tissue-specific promoter, where said promoter has essentially no activity in the leaf epidermis and where the promoter is heterologous with regard to the nucleic acid sequence to be expressed, i.e. does not naturally occur in combination with same. The recombinant expression cassettes according to the invention can optionally comprise further genetic control elements.

Functional linkage is to be understood as meaning, for example, the sequential arrangement of said promoter with the nucleic acid sequence to be expressed and, if appropriate, further regulatory elements such as, for example, a terminator in such a way that each of the regulatory elements can fulfil its function when the nucleic acid sequence is expressed recombinantly. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. A functional linkage as well as a recombinant expression cassette can be generated by means of customary recombination and cloning techniques as are described, for example, in Maniatis T, Fritsch E F and Sambrook J (1989) Molecular Cloning: A Laboratory Manual, Cold Spring Harbor Laboratory, Cold Spring Harbor (NY), in Silhavy T J, Berman M L and Enquist L W (1984) Experiments with Gene Fusions, Cold Spring Harbor Laboratory, Cold Spring Harbor (NY), in Ausubel F M et al. (1987) Current Protocols in Molecular Biology, Greene Publishing Assoc. and Wiley Interscience and in Gelvin et al. (1990) In: Plant Molecular Biology Manual. However, further sequences which, for example, act as a linker with specific cleavage sites for restriction enzymes, or as a signal peptide, may also be positioned between the two sequences. The insertion of sequences may also lead to the expression of fusion proteins.

Preferably, the recombinant expression cassette, consisting of a linkage of promoter and nucleic acid sequence to be expressed, can exist in a vector-integrated form and be inserted into a plant genome, for example by transformation.

However, recombinant expression cassette also denotes those constructions in which the promoter is positioned in front of an endogenous BI1 gene, for example by means of homologous recombination, and thus controls the expression of the BI1 protein. Analogously, the nucleic acid sequence to be expressed (for example coding for a BI1 protein) can also be positioned behind an endogenous promoter in such a way that the same effect is manifested. Both approaches lead to inventive recombinant expression cassettes.

For the purposes of the present invention, a “tissue-specific promoter which has essentially no activity in the leaf epidermis” is generally understood as meaning those promoters which are suitable for ensuring or increasing a recombinant expression of a nucleic acid sequence at least in one plant tissue, with the proviso that
a) said plant tissue is selected from among all plant tissues with the exception of the leaf epidermis, and
b) the recombinant expression under the control of said promoter in said plant tissue amounts to at least five times, preferably at least ten times, especially preferably at least one hundred times the expression in the plant leaf epidermis.

The skilled worker is familiar with a number of promoters which meet these requirements. Especially suitable are tissue-specific promoters such as, by way of example, but not by limitation, promoters with specificity for the anthers, ovaries, flowers, stems, roots, tubers and seeds.
a) Preferred as seed-specific promoters are, for example, the phaseolin promoter (U.S. Pat. No. 5,504,200; Bustos M M et al. (1989). Plant Cell 1(9):839-53), the 2S albumin gene promoter (Joseffson L G et al. (1987) J Biol Chem 262:12196-12201), the legumin promoter (Shirsat A et al. (1989) Mol Gen Genet 215(2): 326-331) and the legumin B4 promoter (LeB4; Baumlein H et al. (1991) Mol Gen Genet 225: 121-128; Baumlein H et al. (1992) Plant J 2(2):233-9; Fiedler U et al. (1995) Biotechnology (NY) 13(10):1090 seq.), the USP (unknown seed protein) promoter (Baumlein H et al. (1991) Mol Gen Genet 225(3):459-67), the napin gene promoter (U.S. Pat. No. 5,608,152; Stalberg K et al. (1996) L Planta 199:515-519), the sucrose binding protein promoter (WO 00/26388) oleosin promoter (WO 98/45461), or the Brassica Bce4 promoter (WO 91/13980). Further suitable seed-specific promoters are those of the genes encoding the high-molecular-weight glutenin (HMWG), gliadin, branching enzyme, ADP glucose pyrophosphatase (AGPase) or starch synthase. Furthermore preferred are promoters which permit seed-specific expression in monocots such as maize, barley, wheat, rye, rice and the like. The following can be employed advantageously: the promoter of the lpt2 or lpt1 gene (WO 95/15389, WO 95/23230) or the promoters described in WO 99/16890 (promoters of the hordein gene, the glutelin gene, the oryzin gene, the prolamin gene, the gliadin gene, the glutelin gene, the zein gene, the kasirin gene or the secalin gene). Further seed-specific promoters are described in WO 89/03887.
b) Tuber-, storage-root- or root-specific promoters comprise, for example, the promoter of the patatin gene (GenBank Acc. No.: A08215), the patatin class I B33 promoter (GenBank Acc. No.: X14483) or the cathepsin D inhibitor promoter from potato. Especially preferred is the promoter described by SEQ ID NO: 29. Tuber-specific promoters are especially suitable for achieving a resistance to Phytophthora infestans in accordance with the invention. Since obligate-biotrophic fungi only attack leaves, an activity in the epidermal tuber tissue is irrelevant.
c) Flower-specific promoters comprise, for example, the phytoene synthase promoter (WO 92/16635) or the promoter of the P-rr gene (WO 98/22593).
d) Anther-specific promoters comprise, for example, the 5126 promoter (U.S. Pat. No. 5,689,049, U.S. Pat. No. 5,689,051), the glob-1 promoter and the γ-zein promoter.
e) Ear-specific promoters such as, for example, the promoter in U.S. Pat. No. 6,291,666. Ear-specific promoters are advantageous in particular for mediating resistance to Fusarium.
f) Mesophyll-specific promoters such as, for example, the promoter of the wheat germin 9f-3.8 gene (GenBank Acc.-No.: M63224) or the barley GerA promoter (WO 02/057412). Said promoters are particularly advantageous since they are not only mesophyll-specific, but also pathogen-inducible. Furthermore suitable are the mesophyll-specific Arabidopsis CAB-2 promoter (GenBank Acc. No.: X15222), and the Zea mays PPCZm1 promoter (GenBank Acc. No.: X63869). Particularly preferred are the promoters described by SEQ ID NO: 30, 31 or 32.

The nucleic acid sequences present in the recombinant expression cassettes or vectors according to the invention can be linked functionally to further genetic control sequences in addition to a promoter. The term “genetic control sequences” is to be understood in the broad sense and refers to all those sequences which have an effect on the materialization or the function of the recombinant expression cassette according to the invention. Genetic control sequences also comprise further promoters, promoter elements or minimal promoters, all of which can modify the expression-governing properties. Thus, for example, the tissue-specific expression may additionally depend on certain stress factors, owing to genetic control sequences. Such elements have been described, for example, for water stress, abscisic acid (Lam E and Chua N H, (1991) J Biol Chem; 266(26): 17131-17135) and heat stress (Schoffl F et al., (1989) Mol Gen Genet 217(2-3):246-53.

Genetic control sequences furthermore also comprise the 5′-untranslated regions, introns or noncoding 3′-region of genes, such as, for example, the actin-1 intron, or the Adh1-S introns 1, 2 and 6 (general reference: The Maize Handbook, Chapter 116, Freeling and Walbot, Eds., Springer, New York (1994)). It has been demonstrated that they may play a significant role in the regulation of gene expression. Thus, it has been demonstrated that 5′-untranslated sequences can enhance the transient expression of heterologous genes. Examples of translation enhancers which may be mentioned are the tobacco mosaic virus 5′ leader sequence (Gallie et al. (1987) Nucl Acids Res 15:8693-8711) and the like. Furthermore, they may promote tissue specificity (Rouster J et al. (1998) Plant J 15:435-440).

The recombinant expression cassette may advantageously comprise one or more of what are known as enhancer sequences, linked functionally to the promoter, which make possible an increased recombinant expression of the nucleic acid sequence. Additional advantageous sequences, such as further regulatory elements or terminators, may also be inserted at the 3′ end of the nucleic acid sequences to be expressed recombinantly. One or more copies of the nucleic acid sequences to be expressed recombinantly may be present in the gene construct.

Polyadenylation signals which are suitable as control sequences are plant polyadenylation signals, preferably those which essentially correspond to T-DNA polyadenylation signals from Agrobacterium tumefaciens, in particular the OCS (octopin synthase) terminator and the NOS (nopalin synthase) terminator.

Control sequences are furthermore to be understood as those which make possible homologous recombination or insertion into the genome of a host organism or which permit removal from the genome. In the case of homologous recombination, for example the natural promoter of a BI1 gene may be exchanged for one of the preferred tissue-specific promoters. Methods such as the cre/lox technology permit a tissue-specific, if appropriate inducible, removal of the recombinant expression cassette from the genome of the host organism (Sauer B (1998) Methods. 14(4):381-92). In this method, specific flanking sequences (lox sequences), which later allow removal by means of cre recombinase, are attached to the target gene.

A recombinant expression cassette and the vectors derived from it may comprise further functional elements. The term functional element is to be understood in the broad sense and refers to all those elements which have an effect on the generation, amplification or function of the recombinant expression cassettes, vectors or recombinant organisms according to the invention. The following may be mentioned by way of example, but not by limitation:
a) Selection markers which confer resistance to a metabolism inhibitor such as 2-deoxyglucose-6-phosphate (WO 98/45456), antibiotics or biocides, preferably herbicides, such as, for example, kanamycin, G 418, bleomycin or hygromycin, or else phosphinothricin and the like. Especially preferred selection markers are those which confer resistance to herbicides. Examples which may be mentioned are: DNA sequences which encode phosphinothricin acetyl transferases (PAT) and which inactivate glutamin synthase inhibitors (bar and pat genes), 5-enolpyruvylshikimate-3-phosphate synthase genes (EPSP synthase genes), which confer resistance to Glyphosat®(N-(phosphonomethyl)glycine), the gox gene, which codes for Glyphosat®-degrading enzymes (Glyphosate oxidoreductase), the deh gene (encoding a dehalogenase which inactivates dalapon®), sulfonylurea- and imidazolinone-inactivating acetolactate synthases, and bxn genes, which encode bromoxynil-degrading nitrilase enzymes, the aasa gene, which confers resistance to the antibiotic apectinomycin, the streptomycin phosphotransferase (SPT) gene, which allows resistance to streptomycin, the neomycin phosphotransferase (NPTII) gene, which confers resistance to kanamycin or geneticidin, the hygromycin phosphotransferase (HPT) gene, which mediates resistance to hygromycin, the acetolactate synthase gene (ALS), which confers resistance to sulfonylurea herbicides (for example mutated ALS variants with, for example, the S4 and/or Hra mutation).
b) Reporter genes which encode readily quantifiable proteins and, via their color or enzyme activity, make possible an assessment of the transformation efficacy, the site of expression or the time of expression. Very especially preferred in this context are reporter proteins (Schenborn E, Groskreutz D. Mol Biotechnol. 1999; 13(1):29-44) such as the green fluorescent protein (GFP) (Sheen et al. (1995) Plant Journal 8(5):777-784; Haseloff et al. (1997) Proc Natl Acad Sci USA 94(6):2122-2127; Reichel et al. (1996) Proc Natl Acad Sci USA 93(12):5888-5893; Tian et al. (1997) Plant Cell Rep 16:267-271; WO 97/41228; Chui W L et al. (1996) Curr Biol 6:325-330; Leffel S M et al. (1997) Biotechniques. 23(5):912-8), chloramphenicol transferase, a luciferase (Ow et al. (1986) Science 234:856-859; Millar et al. (1992) Plant Mol Biol Rep 10:324-414), the aequorin gene (Prasher et al. (1985) Biochem Biophys Res Commun 126(3):1259-1268), β-galactosidase, R locus gene (encoding a protein which regulates the production of anthocyanin pigments (red coloring) in plant tissue and thus makes possible a direct analysis of the promoter activity without addition of further auxiliary substances or chromogenic substrates; Dellaporta et al., In: Chromosome Structure and Function: Impact of New Concepts, 18th Stadler Genetics Symposium, 11:263-282, 1988), with β-glucuronidase being very especially preferred (Jefferson et al., EMBO J. 1987, 6, 3901-3907).
c) Origins of replication, which ensure amplification of the recombinant expression cassettes or vectors according to the invention in, for example, E. coli. Examples which may be mentioned are ORI (origin of DNA replication), the pBR322 ori or the P15A ori (Sambrook et al.: Molecular Cloning. A Laboratory Manual, 2nd ed. Cold Spring Harbor Laboratory Press, Cold Spring Harbor, N.Y., 1989).
d) Elements which are necessary for Agrobacterium-mediated plant transformation, such as, for example, the right or left border of the T-DNA or the vir region.

To select cells which have successfully undergone homologous recombination, or else to select transformed cells, it is, as a rule, necessary additionally to introduce a selectable marker, which confers resistance to a biocide (for example herbicide), a metabolism inhibitor such as 2-deoxyglucose-6-phosphate (WO 98/45456) or an antibiotic to the cells which have successfully undergone recombination. The selection marker permits the selection of the transformed cells from untransformed ones (McCormick et al. (1986) Plant Cell Reports 5:81-84).

The introduction of a recombinant expression cassette according to the invention into an organism or cells, tissues, organs, parts or seeds thereof (preferably into plants or plant cells, tissue, organs, parts or seeds) can be effected advantageously using vectors which comprise the recombinant expression cassettes. The recombinant expression cassette can be introduced into the vector (for example a plasmid) via a suitable restriction cleavage site. The plasmid formed is first introduced into E. coli. Correctly transformed E. coli are selected, grown, and the recombinant plasmid is obtained by the methods familiar to the skilled worker. Restriction analysis and sequencing may serve to verify the cloning step.

Examples of vectors may be plasmids, cosmids, phages, viruses or else agrobacteria. In an advantageous embodiment, the recombinant expression cassette is introduced by means of plasmid vectors. Preferred vectors are those which make possible stable integration of the recombinant expression cassette into the host genome.

The generation of a transformed organism (or of a transformed cell or tissue) requires introducing the DNA, RNA or protein in question into the relevant host cell.

A multiplicity of methods are available for this procedure, which is termed transformation (or transduction or transfection) (Keown et al. (1990) Methods Enzymol 185:527-537); Jenes B et al. (1993) Techniques for Gene Transfer, in: Transgenic Plants, Vol. 1, Engineering and Utilization, published by SD Kung and R Wu, Academic Press, P. 128-143 and in in Potrykus (1991) Annu Rev Plant Physiol Plant Molec Biol 42:205-225).

For example, the DNA or RNA can be introduced directly by microinjection or by bombardment with DNA-coated microparticles. Also, the cell can be permeabilized chemically, for example using polyethylene glycol, so that DNA can enter the cell by diffusion. The DNA can also be introduced by protoplast fusion with other DNA-containing units such as minicells, cells, lysosomes or liposomes. Another suitable method of introducing DNA is electroporation, where the cells are permeabilized reversibly by an electrical pulse. Suitable methods have been described (for example by Bilang et al. (1991) Gene 100:247-250; Scheid et al. (1991) Mol Gen Genet 228:104-112; Guerche et al. (1987) Plant Science 52:111-116; Neuhause et al. (1987) Theor Appl Genet 75:30-36; Klein et al. (1987) Nature 327:70-73; Howell et al. (1980) Science 208:1265; Horsch et al. (1985) Science 227:1229-1231; DeBlock et al. (1989) Plant Physiol 91:694-701).

In plants, the above-described methods of transforming and regenerating plants from plant tissues or plant cells are exploited for transient or stable transformation. Suitable methods are especially protoplast transformation by polyethylene-glycol-induced DNA uptake, the biolistic method with the gene gun, what is known as the particle bombardment method, electroporation, incubation of dry embryos in DNA-containing solution, and microinjection.

In addition to these “direct” transformation techniques, transformation can also be effected by bacterial infection by means of Agrobacterium tumefaciens or Agrobacterium rhizogenes. The Agrobacterium-mediated transformation is best suited to dicotyledonous plant cells. The methods are described, for example, by Horsch R B et al. (1985) Science 225: 1229f).

When agrobacteria are used, the recombinant expression cassette must be integrated into specific plasmids, either into a shuttle or intermediate vector, or into a binary vector. If a Ti or Ri plasmid is to be used for the transformation, at least the right border, but in most cases the right and left border, of the Ti or Ri plasmid T-DNA is linked to the recombinant expression cassette to be introduced in the form of a flanking region.

Binary vectors are preferably used. Binary vectors are capable of replication both in E. coli and in Agrobacterium. As a rule, they comprise a selection marker gene and a linker or polylinker flanked by the right and left T-DNA border sequence. They can be transformed directly into Agrobacterium (Holsters et al. (1978) Mol Gen Genet 163:181-187). The selection marker gene permits a selection of transformed agrobacteria and is, for example, the nptII gene, which confers resistance to kanamycin. The Agrobacterium which acts as host organism in this case should already comprise a plasmid with the vir region. The latter is required for transferring the T-DNA to the plant cell. An Agrobacterium transformed in this way can be used for transforming plant cells. The use of T-DNA for transforming plant cells has been studied and described intensively (EP 120 516; Hoekema, In: The Binary Plant Vector System, Offsetdrukkerij Kanters B. V., Alblasserdam, Chapter V; An et al. (1985) EMBO J 4:277-287). Various binary vectors are known and some commercially available such as, for example, pBI101.2 or pBIN19 (Bevan et al. (1984) Nucl Acids Res 12:8711f; Clontech Laboratories, Inc. USA). Further promoters suitable for expression in plants have been described (Rogers et al. (1987) Methods Enzymol 153:253-277; Schardl et al. (1987) Gene 61:1-11; Berger et al. (1989) Proc Natl Acad Sci USA 86:8402-8406).

Direct transformation techniques are suitable in principle for any organism and cell type. The plasmid used need not meet any particular requirements in the case of the injection or electroporation of DNA or RNA into plant cells. Simple plasmids such as those of the pUC series can be used. If complete plants are to be regenerated from the transformed cells, it is necessary for an additional selectable marker gene to be located on the plasmid.

Stably transformed cells, i.e. those which contain the introduced DNA integrated into the DNA of the host cell, can be selected from untransformed cells when a selectable marker is part of the DNA introduced. Examples of genes which can act as markers are all those which are capable of conferring resistance to antibiotics or herbicides (such as kanamycin, G 418, bleomycin, hygromycin or phosphinothricin) (see above). Transformed cells which express such marker genes are capable of surviving in the presence of concentrations of a corresponding antibiotic or herbicide which kill an untransformed wild type. Examples of suitable selection markers are mentioned above. Once a transformed plant cell has been generated, a complete plant can be obtained using methods known to the skilled worker. For example, callus cultures are used as starting material. The development of shoot and root can be induced in this as yet undifferentiated cell biomass in a known fashion. The plantlets obtained can be planted out and bred. The skilled worker is familiar with methods of regenerating plant parts and intact plants from plant cells. Methods to do so are described, for example, by Fennell et al. (1992) Plant Cell Rep. 11: 567-570; Stoeger et al (1995) Plant Cell Rep. 14:273-278; Jahne et al. (1994) Theor Appl Genet 89:525-533. The resulting plants can be bred and hybridized in the customary fashion. Two or more generations should be grown in order to ensure that the genomic integration is stable and hereditary.

The method according to the invention can advantageously be combined with further methods which bring about pathogen resistance (for example to insects, fungi, bacteria, nematodes and the like), stress resistance or another improvement of the plant properties. Examples are mentioned, inter alia, by Dunwell J M (2000), J Exp Bot. 51 Spec No: 487-96.

The invention furthermore relates to polypeptide sequences coding for a BI1 protein comprising at least one sequence selected from the group consisting of
a) the sequences as shown in SEQ ID NO: 12, 14, 16, 18, 20, 22, 24, 28, 30, 32 or 38,
b) sequences which have least 90%, preferably at least 95%, especially preferably at least 98%, homology with one of the sequences as shown in SEQ ID NO: 12, 14, 16, 18, 20, 22, 24, 28, 30, 32 or 38, and
c) sequences which comprise at least 10, preferably at least 20, especially preferably at least 30, contiguous amino acids of one of the sequences as shown in SEQ ID NO: 12, 14, 16, 18, 20, 22, 24, 28, 30, 32 or 38.

The invention furthermore relates to nucleic acid sequences coding for the novel polypeptide sequences according to the invention which code for BI1 proteins. Preferred are the nucleic acid sequence as shown in SEQ ID NO: 11, 13, 15, 17, 19, 21, 23, 25, 27, 29, 31 or 37, the nucleic acid sequence which is complementary thereto and the sequences derived therefrom as the result of degeneration of the genetic code.

The invention furthermore relates to recombinant expression cassettes which comprise one of the nucleic acid sequences according to the invention. In the recombinant expression cassettes according to the invention, the nucleic acid sequence encoding the barley BI1 protein is linked to at least one genetic control element as defined above in such a manner that it is capable of expression (transcription and, if appropriate, translation) in any organism, preferably in plants. Suitable genetic control elements are described above. The recombinant expression cassettes may also comprise further functional elements in accordance with the above definition. The inserted nucleic acid sequence encoding a barley BI1 protein can be inserted in the expression cassette in sense or antisense orientation and thus lead to the expression of sense or antisense RNA. Recombinant vectors comprising the recombinant expression cassettes are also in accordance with the invention.

“Recombinant”, for example regarding a nucleic acid sequence, an expression cassette or a vector comprising said nucleic acid sequence or an organism transformed with said nucleic acid sequence, expression cassette or vector, refers to all those constructs originating by genetic engineering methods in which either
a) the BI1 nucleic acid sequence, or
b) a genetic control sequence linked functionally to the BI1 nucleic acid sequence, for example a promoter, or
c) (a) and (b)
   are not located in their natural genetic environment or have been modified by recombinant methods, an example of a modification being substitutions, additions, deletions, inversions or insertions of one or more nucleotide residues. Natural genetic environment refers to the natural chromosomal locus in the organism of origin, or to the presence in a genomic library. In the case of a genomic library, the natural genetic environment of the nucleic acid sequence is preferably retained, at least in part. The environment flanks the nucleic acid sequence at least at one side and has a sequence of at least 50 bp, preferably at least 500 bp, especially preferably at least 1000 bp, very especially preferably at least 5000 bp, in length. A naturally occurring expression cassette—for example the naturally occurring combination of the BI1 promoter with the corresponding BI1 gene—becomes a recombinant expression cassette when it is modified by non-natural, synthetic (“artificial”) methods such as, for example, mutagenization.

Such methods have been described (U.S. Pat. No. 5,565,350; WO 00/15815; also see above).

The invention also relates to recombinant organisms transformed with at least one of the nucleic acid sequences according to the invention, expression cassette according to the invention or vector according to the invention, and to cells, cell cultures, tissues, parts—such as, for example, leaves, roots and the like in the case of plant organisms—or propagation material derived from such organisms. The term organism is to be understood in the broad sense and refers to prokaryotic and eukaryotic organisms, preferably bacteria, yeasts, fungi, animal organisms and plant organisms. Host organisms, or starting organisms, which are preferred as recombinant organisms are in particular plants as defined above.

The invention furthermore relates to the use of the recombinant organisms according to the invention and of the cells, cell cultures, parts—such as, for example, roots, leaves and the like in the case of recombinant plant organisms—derived from them, and to recombinant propagation material such as seeds or fruits, for the production of foodstuffs or feeding stuffs, pharmaceuticals or fine chemicals.

Furthermore a nucleic acid molecule which is antisense to the nucleic acid according to the invention, is a monoclonal antibody which binds specifically to the polypeptide according to the invention and a fungicide which comprises the nucleic acid according to the invention, the vector according to the invention, in particular an infectious, for example viral, vector according to the invention, the polypeptide according to the invention in a form which is suitable for application to plants, for example in encapsulated form or in an infectious organism preferably suitable for transferring nucleic acids or expressing genes in a cell, such as an Agrobacterium or a virus.

In one embodiment, the invention relates to the use of a nucleic acid molecule which codes for BI-1, or of a BI-1 protein, for the generation of a pathogen-resistant plant, preferably for the generation of a plant which is resistant to fungi or for the generation of a fungicide bringing about the same, or for controlling or treating plants which are attacked, or liable to attack, by pathogens.

Sequences

1. SEQ ID NO: 1: Nucleic acid sequence coding for a BI1 protein from barley (Hordeum vulgare).
2. SEQ ID NO: 2: Amino acid sequence coding for a BI1 protein from barley (Hordeum vulgare).
3. SEQ ID NO: 3: Nucleic acid sequence coding for a BI1 protein from Arabidopsis thaliana.
4. SEQ ID NO: 4: Amino acid sequence coding for a BI1 protein from Arabidopsis thaliana.
5. SEQ ID NO: 5: Nucleic acid sequence coding for a BI1 protein from tobacco.
6. SEQ ID NO: 6: Amino acid sequence coding for a BI1 protein from tobacco.
7. SEQ ID NO: 7: Nucleic acid sequence coding for a BI1 protein from rice.
8. SEQ ID NO: 8: Amino acid sequence coding for a BI1 protein from rice.
9. SEQ ID NO: 9: Nucleic acid sequence coding for a BI1 protein from oilseed rape.
10. SEQ ID NO: 10: Amino acid sequence coding for a BI1 protein from oilseed rape.
11. SEQ ID NO: 11: Nucleic acid sequence coding for a part of a BI1 protein from soybean.
12. SEQ ID NO: 12: Amino acid sequence coding for a part of a BI1 protein from soybean.
13. SEQ ID NO: 13: Nucleic acid sequence coding for a part of a BI1 protein from soybean.
14. SEQ ID NO: 14: Amino acid sequence coding for a part of a BI1 protein from soybean.
15. SEQ ID NO: 15: Nucleic acid sequence coding for a part of a BI1 protein from wheat.
16. SEQ ID NO: 16: Amino acid sequence coding for a part of a BI1 protein from wheat.
17. SEQ ID NO: 17: Nucleic acid sequence coding for a part of a BI1 protein from maize.
18. SEQ ID NO: 18: Amino acid sequence coding for a part of a BI1 protein from maize.
19. SEQ ID NO: 19: Nucleic acid sequence coding for a part of a BI1 protein from wheat.
20. SEQ ID NO: 20: Amino acid sequence coding for a part of a BI1 protein from wheat.
21. SEQ ID NO: 21: Nucleic acid sequence coding for a part of a BI1 protein from maize.
22. SEQ ID NO: 22: Amino acid sequence coding for a part of a BI1 protein from maize.
23. SEQ ID NO: 23: Nucleic acid sequence coding for a part of a BI1 protein from maize.
24. SEQ ID NO: 24: Amino acid sequence coding for a part of a BI1 protein from maize.
25. SEQ ID NO: 25: Nucleic acid sequence coding for a part of a BI1 protein from wheat.
26. SEQ ID NO: 26: Amino acid sequence coding for a part of a BI1 protein from wheat.
27. SEQ ID NO: 27: Nucleic acid sequence coding for a part of a BI1 protein from maize.
28. SEQ ID NO: 28: Amino acid sequence coding for a part of a BI1 protein from maize.
29. SEQ ID NO: 29: Nucleic acid sequence coding for the patatin promoter from potato.
30. SEQ ID NO: 30: Nucleic acid sequence coding for the germin 9f-3.8 promoter from wheat.
31. SEQ ID NO: 31: Nucleic acid sequence coding for the Arabidopsis CAB-2 promoter.
32. SEQ ID NO: 32: Nucleic acid sequence coding for the PPCZm1 promoter from maize.
33. SEQ ID NO: 33: Nucleic acid sequence coding for the recombinant expression vector pUbiBI-1.
34. SEQ ID NO: 34: Nucleic acid sequence coding for the recombinant expression vector pLo114UbiBI-1.
35. SEQ ID NO: 35: Nucleic acid sequence coding for the recombinant expression vector pOXoBI-1.
36. SEQ ID NO: 36: Nucleic acid sequence coding for the recombinant expression vector pLo114OXoBI-1.
37. SEQ ID NO: 37: Nucleic acid sequence coding for BI-1 protein from wheat.
38. SEQ ID NO: 38: Amino acid sequence coding for a BI1 protein from wheat.
39. SEQ ID NO: 39: Nucleic acid sequence for PEN1 (=ROR2) from barley.
40. SEQ ID NO: 40: Amino acid sequence coding for PEN1 (=ROR2) from barley.
41. SEQ ID NO: 41: Nucleic acid sequence for PEN1 (=ROR2) from Arabidopsis thaliana.
42. SEQ ID NO: 42: Amino acid sequence coding for PEN1 (=ROR2) from Arabidopsis thaliana.
43. SEQ ID NO: 43: Nucleic acid sequence coding for SNAP34 from barley.
44. SEQ ID NO: 44: Amino acid sequence coding for SNAP34 from barley.

### FIGURES

1. FIG. 1a-d: Alignment of protein sequences of different BI-1 proteins from plants. AtBI-1: Arabidopsis; BnBI-1: Brassica napus (oilseed rape); GmBI2: Glycine max (soybean; variant 1); GmBI3: Glycine max (soybean; variant 2); HVBI-1: Hordeum vulgare (barley); NtBI-1: Nicotiana tabacum (tobacco); OsBI-1: Oryza sativa (rice); TaBI11: Triticum aestivum (wheat, variant 1); TaBI18: Triticum aestivum (wheat, variant 2); TaBI5 new: Triticum aestivum (wheat, variant 3); ZmBI14: Zea mays (maize; variant 1); ZmBI16: Zea mays (maize; variant 2); ZmBI33: Zea mays (maize; variant 3); ZmBI8: Zea mays (maize; variant 4); Consensus: consensus sequence derived from the alignment.

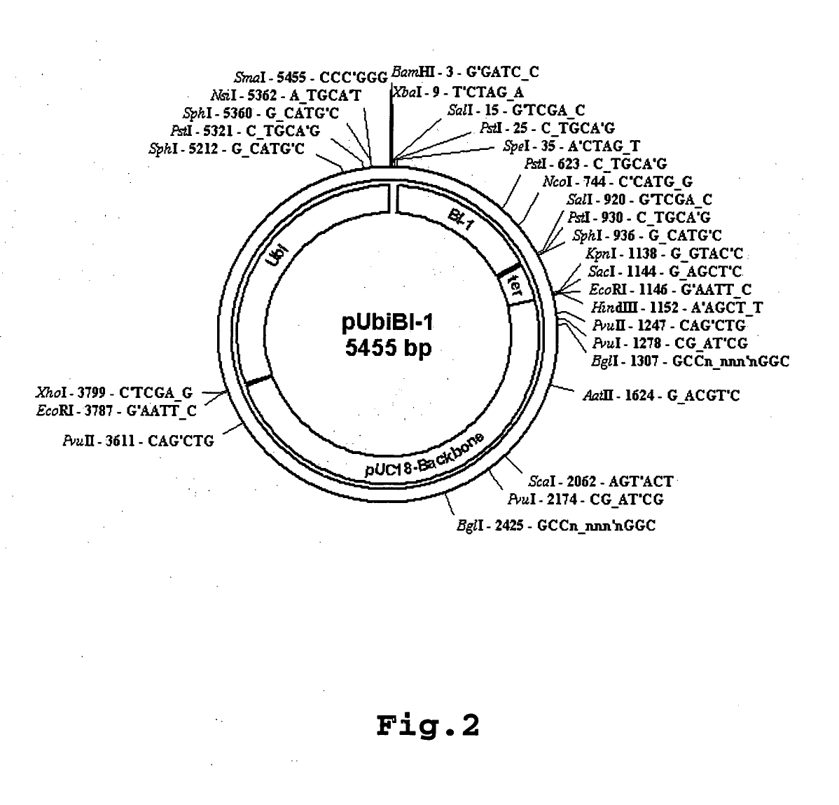

2. FIG. 2: Vector map for the vector pUbiBI-1 (Ubi: ubiquitin promoter; BI-1 nucleic acid sequence coding for barley BI1 protein; ter: transcription terminator). Also shown are the localizations of the cleavage sites for different restriction enzymes.

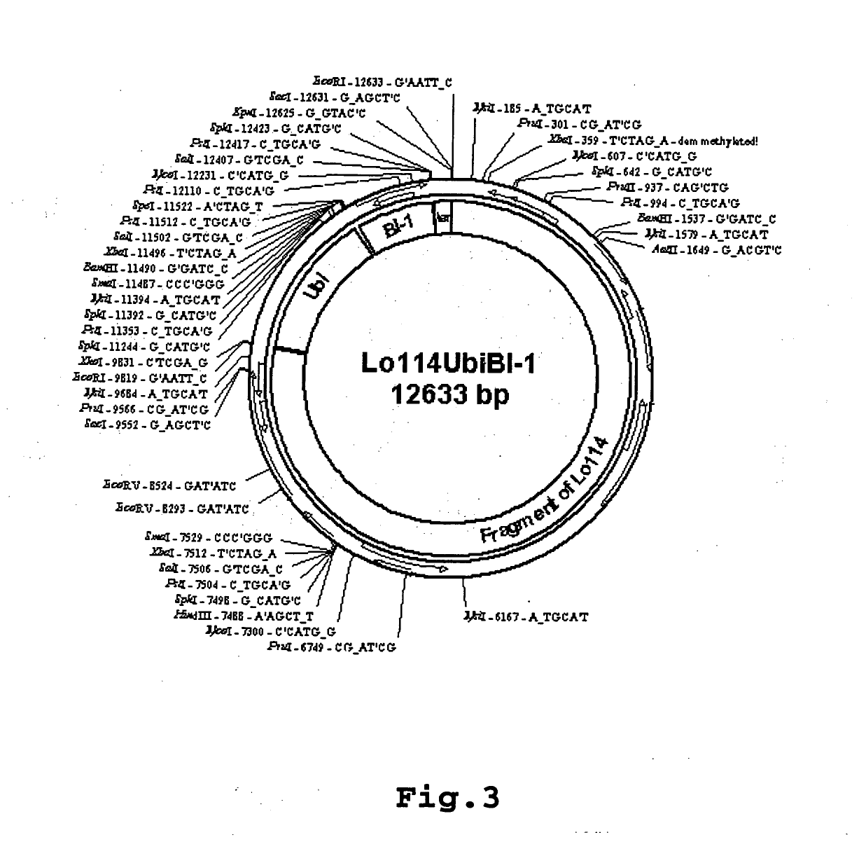

3. FIG. 3: Vector map for the vector pLO114UbiBI-1 (Ubi: ubiquitin promoter; BI-1 nucleic acid sequence coding for barley BI1 protein; ter: transcription terminator). Also shown are the localizations of the cleavage sites for different restriction enzymes.

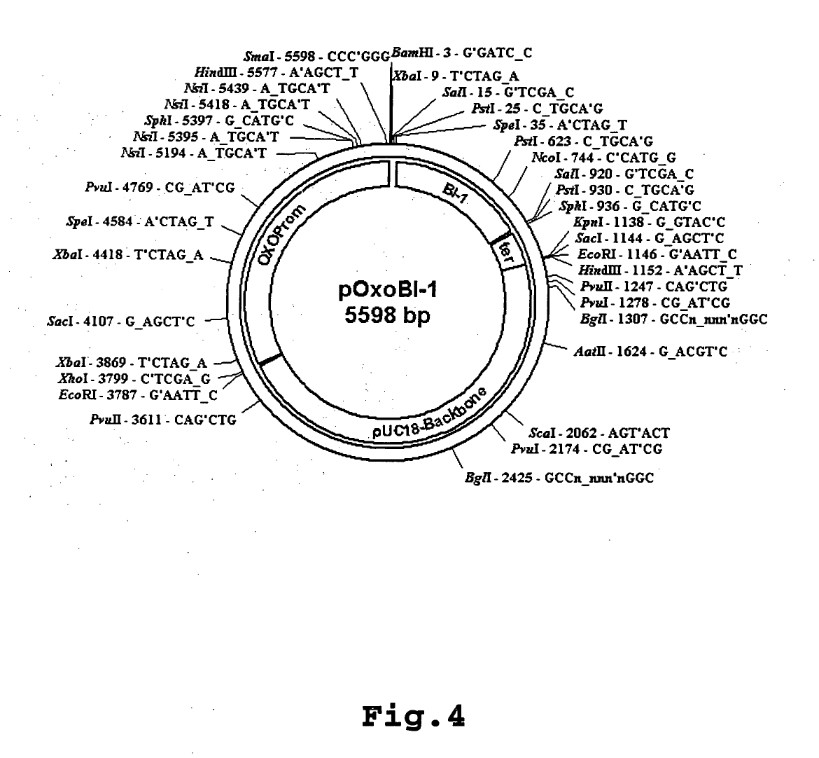

4. FIG. 4: Vector map for the vector pOxoBI-1 (Oxo: TaGermin 9f-2.8 promoter; BI-1 nucleic acid sequence coding for barley BI1 protein; ter: transcription terminator). Also shown are the localizations of the cleavage sites for different restriction enzymes.

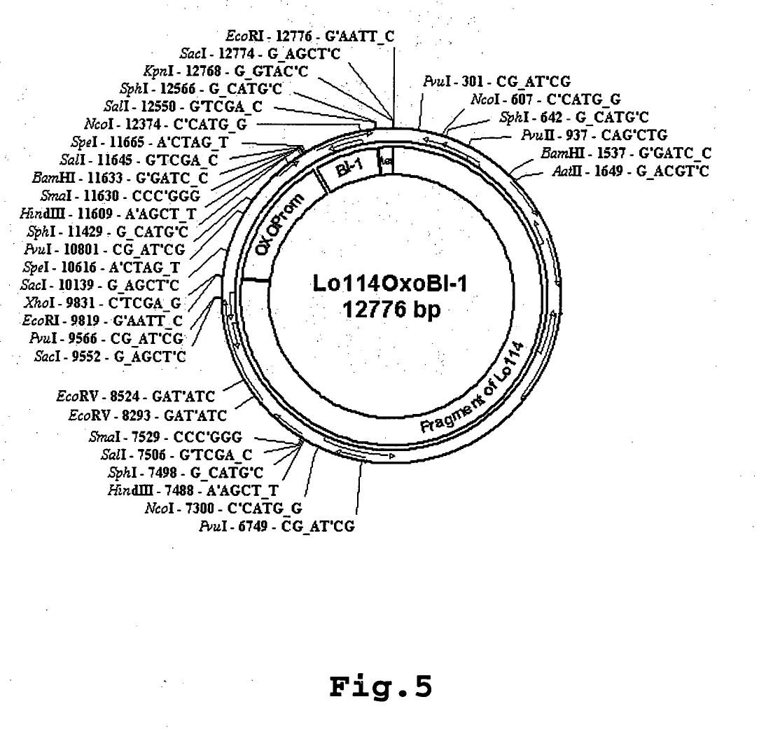

5. FIG. 5: Vector map for the vector pLO114OxoBI-1 (Oxo: TaGermin 9f-2.8 promoter; BI-1 nucleic acid sequence coding for barley BI1 protein; ter: transcription terminator). Also shown are the localizations of the cleavage sites for different restriction enzymes.

6. FIG. 6: Alignment of the protein sequences of BI-1 proteins from barley (Hordeum vulgare, GenBank Acc. No.: CAC37797), rice (Oryza sativa, GenBank Acc. No.: Q9MBD8), Arabidopsis thaliana (GenBank Acc. No.: Q9LD45) and humans (Homo sapiens, GenBank Acc. No.: AAB87479). Amino acids shown against the black background are identical in all species. Amino acids shown against the gray background are identical in plants only. Bars indicate the predicted seven transmembrane domains in HvBI-1.

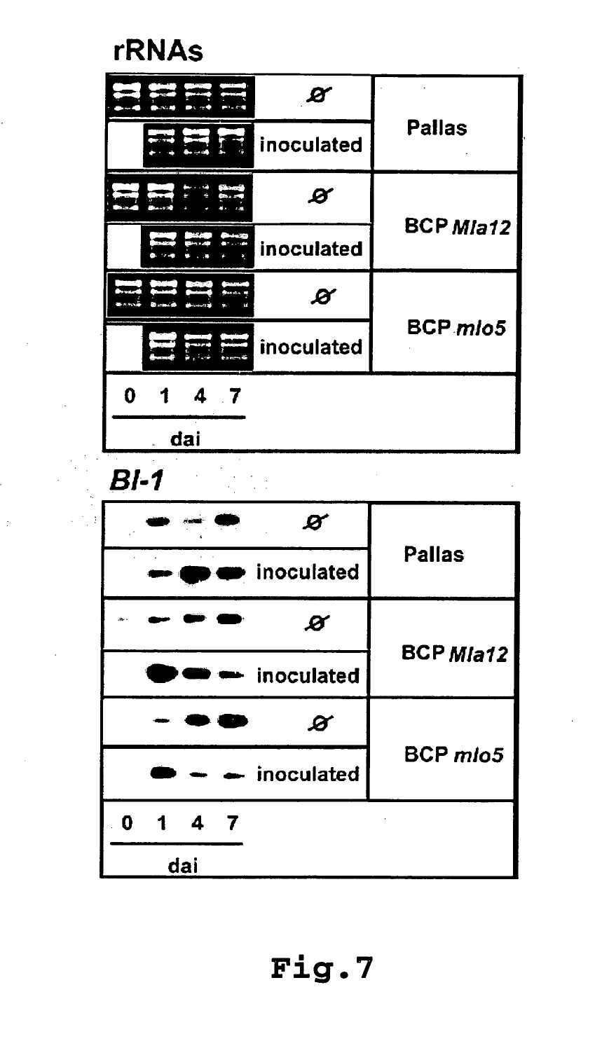

7. FIG. 7: BI-1 expression in resistant and susceptible barley lines (cDNA gel blot analysis): cDNAs were synthesized by means of RT-PCR, starting from total RNA. Total RNA was obtained from the susceptible barley line Pallas, the resistant barley line BCPM1a12 and the resistant barley line BCPm1o5 at times 0 (i.e. immediately prior to inoculation) and in each case 1, 4 and 7 days after inoculation with Bgh and, in parallel, from uninfected control plants (Ø). The RT-PCR for BI-1 was carried out using 20 cycles (see hereinbelow). The amount of RNA employed (0.5 μg) was additionally checked in gels by means of rRNA staining with ethidium bromide. A repetition of the experiments gave comparable results.

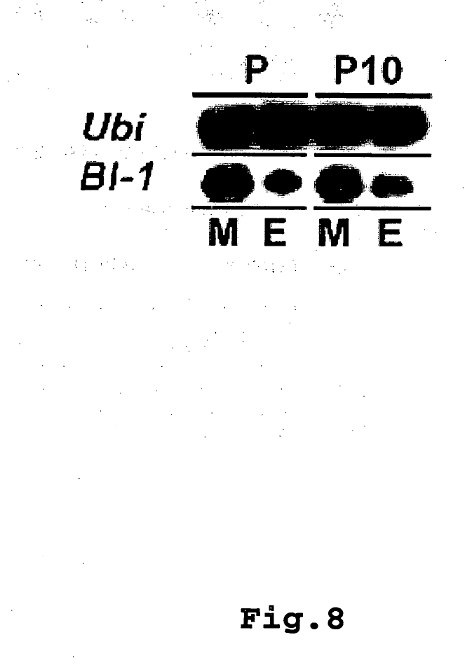

8. FIG. 8: BI-1 is expressed in mesophyll tissue (cDNA gel blot analysis). RT-PCR was carried out starting from RNA isolated from Pallas (P) and BCPM1a12 (P10) (24 h after inoculation with BghA6). To extract the total RNA, abaxial epidermal strips (E, inoculated positions of the leaves) were separated from the mesophyll and the adaxial epidermis (M). Ubiquitin 1 (Ubi) was used as label for tissue-unspecific gene expression. RT-PCR was carried out using 30 cycles.

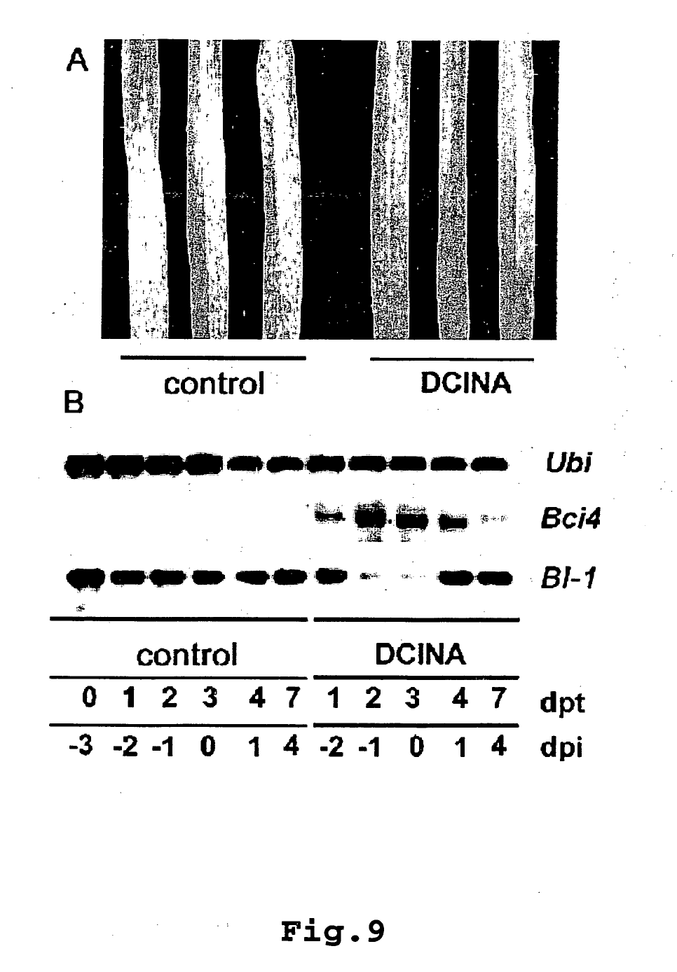

9. FIG. 9: BI-1 expression is repressed during chemical resistance induction.

(A) Chemical induced resistance in the barley line Pallas gg. Blumeria graminis (DC) Speer f.sp. hordei (Bgh). Barley primary leaves were treated with 2,6-dichloroisonicotinic acid (DCINA) and showed fewer mildew pustules than corresponding untreated control plants.

(B) RNA and cDNA Blots. RNA (10 μg) was analyzed 0, 1, 2 and 3 days after soil treatment (soil drench treatment; dpt) with DCINA and with the control (carrier substance) and additionally 1 and 4 days post-inoculation (dpi, corresponds to 4 and 7 dpt, respectively). RT-PCR (Ubi, BI-1) was carried out using 20 cycles. Repetition resulted in comparable results (see Example 2).

BCI-4 was employed as the control. BCI-4 is a DCINA-induced gene (Besser et al. (2000) Mol Plant Pahol. 1(5): 277-286) and a member of the Barley Chemically (=BTH) induced gene family.

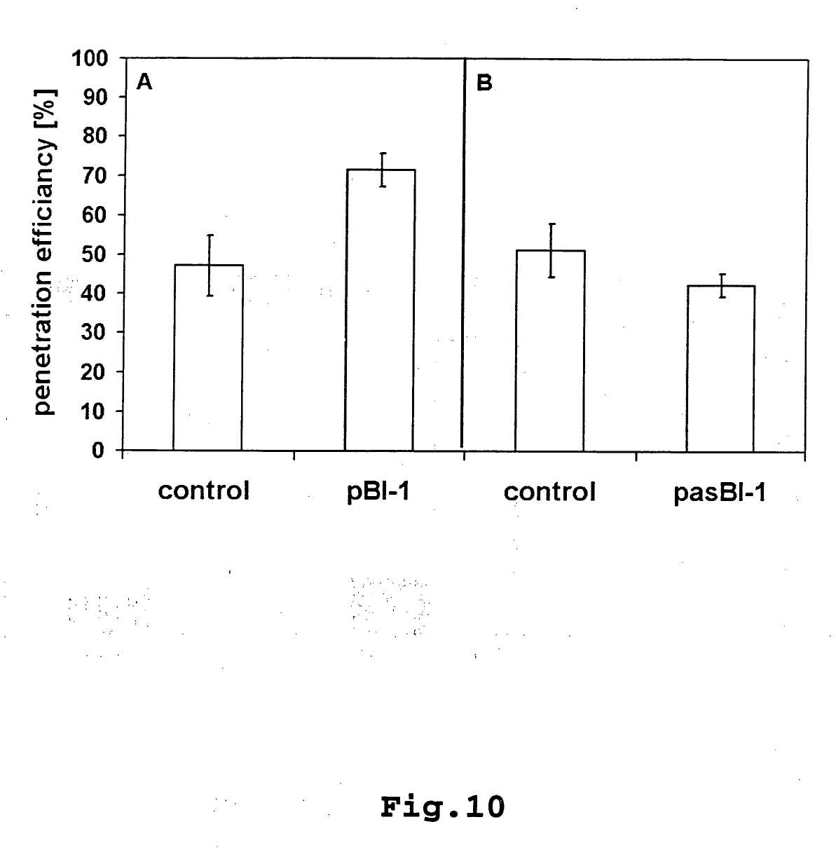

10. FIG. 10: Overexpression of BI-1 induced supersusceptibility.

(A) Mean penetration efficiency of Bgh in 6 independent experiments with Bgh on barley line Ingrid. The PE of Bgh was significantly increased (p<0.01, Student's t-test) in cells which were transformed with pBI-1 (by bombardment) in comparison with cells which were bombarded with the blank vector control (pGY1).

(B) The penetration efficiency of Bgh on cells which had been bombarded with an antisense BI-1 construct (pasBI-1) was not significantly reduced (p>0.05) in comparison with cells which had been bombarded with the blank vector control (pGY1).

The columns show in each case the mean value of the individual experiments. The bars represent the standard error.

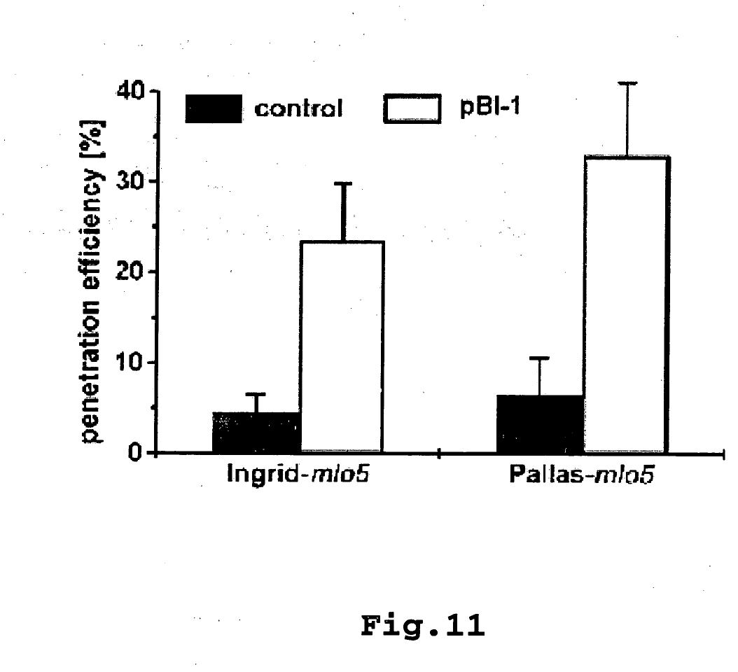

11. FIG. 11: Overexpression of BI-1 induced breaking of the m1o5-mediated penetration resistance.

The penetration efficiency of Bgh was assessed in 3 to 4 independent experiments using Bgh on the barley lines Ingrid-m1o5 and pallas-m1o5. The PE caused by Bgh was significantly increased (p<0.05) in cells which had been transformed with pBI-1 (bombarded) in comparison with cells which had been bombarded with the blank vector control (pGY1). The columns show in each case the mean value of three independent experiments. The bars represent the standard error.

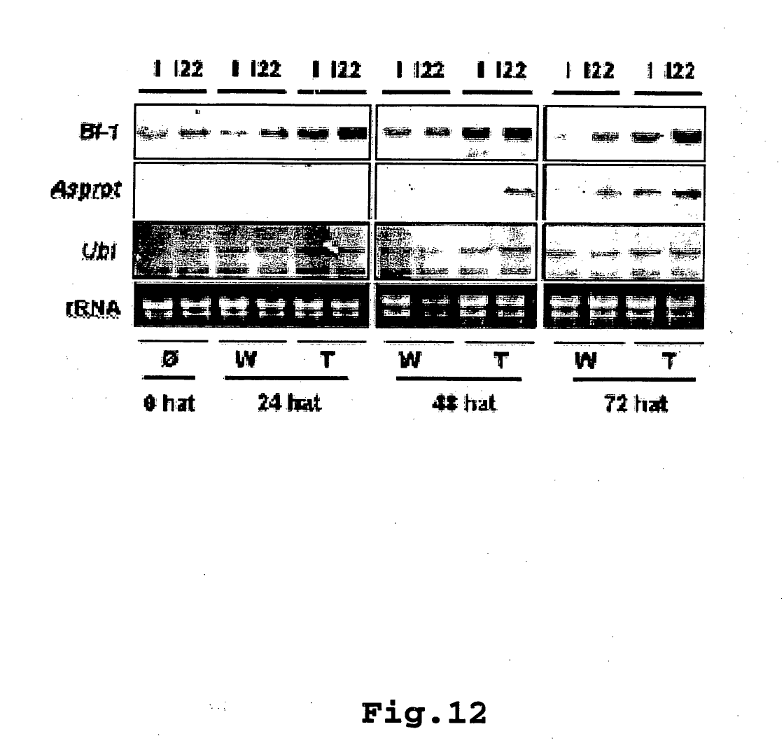

12. FIG. 12: The expression of BI-1 is induced by toxic culture filtrates from Bipolaris sorokiniana. Northern blots (10 μg total RNA) with RNA from Ingrid (I) and BCIngrid-m1o5 (122). RNA was isolated 0, 24, 48 and 72 hours after injection of the toxic culture filtrates of Bipolaris sorokiniana (T) or water (W). BI-1 mRNAs were detected on nylon membranes following stringent washing. BI-1: detection of BAX Inhibitor 1 mRNA; Ubi: detection of Ubiquitin 1; Asprot: detection of the aspartate protease mRNA; hat: hours after treatment (“h after treatment”).

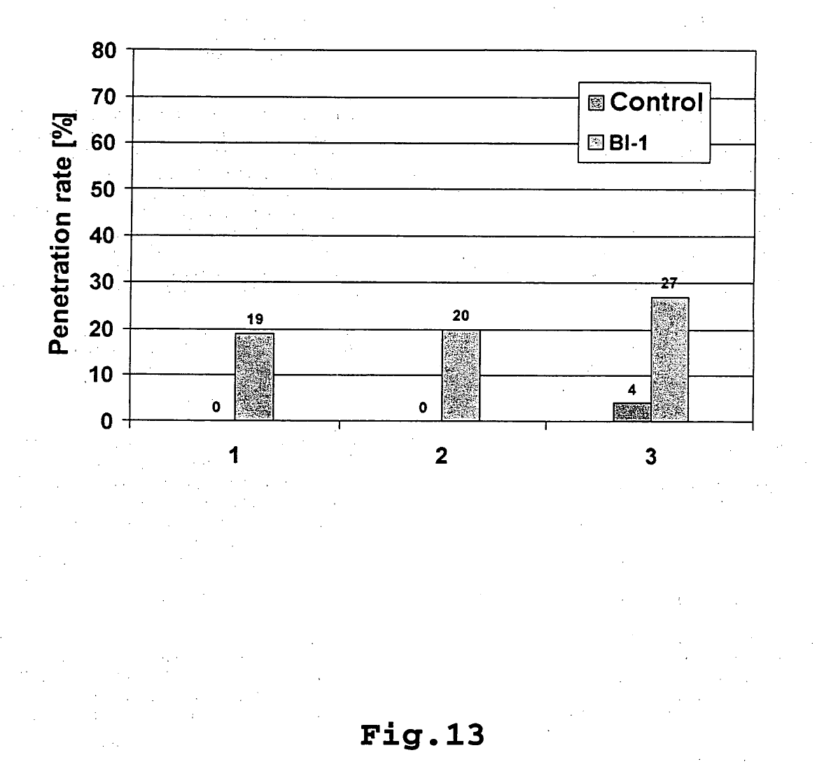

13. FIG. 13: BI-1 overexpression breaks non-host resistance of barley (cv. Manchuria) to Blumeria graminis f.sp. tritici. The penetration rates were analyzed in three independent experiments.

### EXAMPLES

General methods:

The chemical synthesis of oligonucleotides can be effected, for example, in the known fashion using the phosphoamidite method (Voet, Voet, 2nd Edition, Wiley Press New York, pages 896-897). The cloning steps carried out for the purposes of the present invention such as, for example, restriction cleavages, agarose gel electrophoresis, purification of DNA fragments, transfer of nucleic acids to nitrocellulose and nylon membranes, linking DNA fragments, transformation of E. coli cells, growing bacteria, multiplying phages and sequence analysis of recombinant DNA, are carried out as described by Sambrook et al. (1989) Cold Spring Harbor Laboratory Press; ISBN 0-87969-309-6. The sequencing of recombinant DNA molecules is carried out with an MWG-Licor laser fluorescence DNA sequencer following the method of Sanger (Sanger et al. (1977). Proc Natl Acad Sci USA 74:5463-5467).

### Example 1

### Plants, Pathogens and Inoculation

The barley varieties Ingrid, Pallas and the backcrossed line BCPM1a12, BCPm1o5 and BCIngrid-m1o5 (I22) were donated by Lisa Munk, Department of Plant Pathology, Royal Veterinary and Agricultural University, Copenhagen, Denmark. Its production has been described (Kølster P et al. (1986) Crop Sci 26: 903-907).

Unless otherwise specified, the seed which had been pregerminated on moist filter paper for 12 to 36 hours in the dark was sown along the edge of a square pot (8×8 cm; 5 kernels per pot) in Fruhstorfer soil, type P, covered with soil and watered regularly with tap water. All of the plants were cultured in controlled-environment cabinets or chambers for 5 to 8 days at 18° C., 60% relative atmospheric humidity and a 16-hr-light/8-hr-dark rhythm at 3000 and 5000 lux, respectively (photon flow density 50 and 60 μmols-¹m-^{2·}; respectively) and used in the experiments during the seedling stage. In experiments in which applications to the primary leaves were carried out, the latter were developed fully.

Before the transient transfection experiments were carried out, the plants were grown in controlled-environment cabinets or chambers at a daytime temperature of 24° C., a nighttime temperature of 20° C., 50 to 60% relative atmospheric humidity and a 16-hr-light/8-hr-dark rhythm at 30 000 lux.

Barley powdery mildew Blumeria graminis (DC) Speer f.sp. hordei Em. Marchal race A6 (Wiberg A (1974) Hereditas 77: 89-148) (BghA6) was used for the inoculation of barley plants. The fungus was provided by the Department of Biometry, JLU Gieβen. Inoculum was maintained in controlled-environment cabinets under identical conditions to those described above for the plants by transferring the conidia of infected plant material at a rate of 100 conidia/mm² to 7-day-old barley plants cv. Golden Promise, which were grown regularly.

Inoculation was carried out on primary leaves of barley plants with the following conidial densities: 5 conidia/mm² in the case of chemical resistance induction and macroscopic evaluation of the induction success, 50 conidia/mm² in the case of gene expression studies and 150 conidia/mm² for the verification of the gene transformation using transformed leaf segments. The inoculation with BghA6 was carried out using 7-day-old seedlings by shaking off the conidia of already infected plants in an inoculation tower (unless otherwise specified).

### Example 2

### Modulation of the Expression of BI1 using DCINA

2,6-Dichloroisonicotinic acid (DCINA, Syngenta A G, Basle, Switzerland; as a 25% (w/w) formulation) was applied to 4-day-old barley seedlings cv. Pallas by means of soil drench at a final concentration of 8 mg/l soil volume. The suspension used was made with tap water. Soil drench with the carrier material (wettable powder) acted as the control. After three days, the plants were infected with Blumeria graminis (DC) Speer f.sp. hordei Em. Marchal, race A6 (5 conidia/mm²). Plants with chemically induced resistance (CIR) showed approximately 70% fewer mildew colonies than the corresponding control plants which had only been treated with the carrier substance (FIG. 9A).

To determine the amounts of BI1 transcripts, Northern blot and RT-PCT blots were carried out; they revealed a surprising reduction of the expression of BI1 1 to 3 days after the chemical treatment (FIG. 9B).

### Example 3

### RNA Extraction

Total RNA was extracted from 8 to 10 primary leaf segments (length 5 cm) by means of “RNA extraction buffer” (AGS, Heidelberg, Germany). To this end, the central primary leaf segments 5 cm in length were harvested and homogenized in liquid nitrogen in mortars. The homogenate was stored at −70° C. until the RNA was extracted. Total RNA was extracted from the deep-frozen leaf material with the aid of an RNA extraction kit (AGS, Heidelberg). To this end, 200 mg of the deep-frozen leaf material were covered with 1.7 ml RNA extraction buffer (AGS) in a microcentrifuge tube (2 ml) and immediately mixed thoroughly. After addition of 200 μl of chloroform, the mixture was again mixed thoroughly and shaken for 45 minutes on a horizontal shaker at 200 rpm at room temperature. To separate the phases, the tubes were subsequently centrifuged for 15 minutes at 20 000 g and 4° C., and the upper, aqueous phase was transferred into a fresh microcentrifuge tube, while the bottom phase was discarded. The aqueous phase was repurified with 900 μl of chloroform by homogenizing for 10 seconds and recentrifuging (see above) and removing the aqueous phase (3 times). Then, 850 μl of 2-propanol were added and the mixture was homogenized and placed on ice for 30 to 60 minutes in order to precipitate the RNA. Thereafter, the mixture was centrifuged for 20 minutes (see above), the supernatant was carefully decanted off, 2 ml of 70% strength ethanol (−20° C.) were pipetted in, and the mixture was mixed and recentrifuged for 10 minutes. Then, the supernatant was again decanted off, and the pellet was carefully freed from residual fluid, using a pipette, and then dried in a stream of clean air on a clean bench. Then, the RNA was dissolved in 50 μl of DEPC water on ice, mixed and centrifuged for 5 minutes (see above). 40 μl of the supernatant, constituting the RNA solution, were transferred into a fresh microcentrifuge tube and stored at −70° C.

The RNA concentration was determined photometrically. To this end, the RNA solution was diluted 1:99 (v/v) with distilled water, and the absorption was measured at 260 nm (Beckman Photometer DU 7400); (E₂₆₀ nm=1 at 40 μg RNA/ml). The concentrations of the RNA solutions were subsequently adjusted to 1 ∝g/∝l with DEPC water to match the calculated RNA contents and verified in an agarose gel.

To verify the RNA concentrations in a horizontal agarose gel (1% agarose in 1×MOPS buffer with 0.2 μg/ml ethidium bromide), 1 μl of RNA solution was treated with 1 μl of 10×MOPS, 1 μl of color marker and 7 μl of DEPC water, separated according to size in 1×MOPS running buffer over 1.5 hours at a voltage of 120 V in the gel, and photographed under UV light. Any differences in concentration of the RNA extracts were adjusted with DEPC water, and the adjustment was rechecked in the gel.

### Example 4

### Cloning the BI1 cDNA Sequence from Barley

The full-length clone of hvBI1 (GenBank Acc.-No.: AJ290421) comprises two stop codons at the 3′ end and a potential start codon at the 5′ end. The ORF spans 247 amino acids and shows the highest degree of sequence homology with a BI1 gene from rice, maize, Brassica napus and Arabidopsis thaliana (in each case 86% identity at the nucleotide level) and a human BI1 homolog (53% similarity) (FIGS. 1 and 6). The amino acid sequence of hvBI1 comprises seven potential transmembrane domains with an orientation of the C terminus in the cytosol.

The following constructs were prepared:

a) Amplification of a 478 bp fragment of the barley BI1 cDNA (GenBank Acc.-No.: AJ290421)

| | |
|---|---|
| BI1-sense | 5′-atggacgccttctactcgacctcg-3′ |
| BI1-antisense | 5′- gccagagcaggatcgacgcc-3′ |

b) Amplification of a 513 bp Ubi cDNA fragment (GenBank Acc.-No.: M60175)

| | |
|---|---|
| UBI-sense | 5′-ccaagatgcagatcttcgtga-3′ |
| UBI-antisense | 5′-ttcgcgataggtaaaagagca-3′ |

c) Amplification of an 871 bp full-length BI1 reading frame

| | |
|---|---|
| BI1VL sense | 5′-ggattcaacgcgagcgcaggacaagc-3′ |
| BI1VL antisense | 5′-gtcgacgcggtgacggtatctacatg-3′ |

The fragments obtained were ligated into the vector pGEM-T by means of T-overhang ligation and acted as starting plasmids for the generation of probes (for example for Northern blot) or dsRNA. The individual constructs were referred to as pGEMT-BI1, pGEMT-BI1VL(240) and pGEMT-UBI.

The BI1 full-length product was recloned from pGEMT into the SalI cleavage site of the pGY-1 vector (Schweizer, P., Pokorny, J., Abderhalden, O. & Dudler, R. (1999) Mol. Plant-Microbe Interact. 12, 647-654) using the SalI cleavage site in pGEMT and by means of the SalI cleavage sites which had been attached to the BI1VL antisense primer. Vectors with sense (pBI-1) and antisense (pasBI-1) orientation were isolated and resequenced. The vectors comprise the BI-1 sequence under the control of the CaMV 35S promoter.

### Example 5

### Reverse Transcription—Polymerase Chain Reaction (RT-PCR)

To detect small amounts of transcript, a semiquantiative RT-PCR was carried out using the “OneStep RT-PCR Kit” (Qiagen, Hilden, Germany). In doing so, RNA (isolated as above) was first translated into cDNA (reverse transcription) and the sought cDNA was amplified in a subsequent PCR reaction using specific primers. To estimate the initial amount of template RNA, the amplification was interrupted during the exponential phase (after 20 cycles) in order to reflect differences in the target RNA. The PCR products were separated by means of an agarose gel, denatured, blotted onto nylon membranes, and detected with specific non-radiolabeled probes under stringent standard conditions. Hybridization, wash steps and immunodetection were carried out as described under “Northern blot”. The following components were combined for the individual reactions (25 μl batch) using the “One Step RT-PCR Kit” (Qiagen, Hilden, Germany):
1000 ng total RNA of a specific sample
0.4 mM dNTPs
0.6 μM of each sense and antisense primer
0.10 μl RNase inhibitor
1 μl enzyme mix in 1×RT buffer.

cDNA synthesis (reverse transcription) was carried out for 30 minutes at 50° C. The reverse transcriptase was subsequently inactivated for 15 minutes at 95° C., which simultaneously causes activation of DNA polymerase and denaturation of cDNA. A PCR was subsequently carried out with the following program: 1 minute at 94° C.; 25 cycles of 1 minute at 94° C.; 1 minute at 54° C. and 1 minute at 72° C.; 10 minutes at 72° C. completion. Then storage at 4° C. until further use. The PCR products were separated in a 1×TBE agarose gel using ethidium bromide. The above primer pairs were used for the amplifications in the individual batches.

### Example 6

### Northern Blot Analysis

To prepare the Northern blotting, the RNA was separated in agarose gel under denaturing conditions. To this end, part of the RNA solution (corresponding to 10 μg of RNA) was mixed with an identical volume of sample buffer (with ethidium bromide), denatured for 5 minutes at 94° C., placed on ice for 5 minutes, centrifuged briefly and applied to the gel. The 1×MOPS gel (1.5% agarose, ultra pure grade) comprised 5 percent by volume of concentrated formaldehyde solution (36.5% [v/v]). The RNA was separated for 2 hours at 100 V and subsequently blotted.

Northern blotting was done as an upward capillary RNA transfer. To this end, the gel was first agitated gently for 30 minutes in 25 mM sodium hydrogen/dihydrogen phosphate buffer (pH 6.5) and cut to size. A piece of Whatman paper was prepared in such a way that it rested on a horizontal slab and extended on 2 sides into a trough with 25 mM sodium hydrogen/dihydrogen phosphate buffer (pH 6.5). This piece of paper was covered with the gel, uncovered parts of the piece of Whatman paper being covered with a plastic film. The gel was then covered with a positively charged nylon membrane (Boehringer-Mannheim), avoiding air bubbles, whereupon the membrane was recovered to a height of approximately 5 cm with a stack of blotting paper. The blotting paper was additionally weighed down with a sheet of glass and with a 100 g weight. Blotting was carried out overnight at room temperature. The membrane was rinsed briefly in twice-distilled water and irradiated with UV light in a crosslinking apparatus (Biorad) with a light energy of 125 mJ in order to immobilize the RNA. The uniformity of the RNA transfer to the membrane was checked on a UV-light bench.

To detect barley mRNA, 10 mg of total RNA from each sample were resolved in an agarose gel and blotted onto a positively charged nylon membrane by capillary transfer. Detection was effected using the DIG system according to manufacturing specifications with digoxygenin-labeled antisense RNA probes (as described in Hückelhoven R et al. (2001) Plant Mol Biol 47:739-748).

Probe preparation: Digoxygenin- or fluorescein-labeled RNA probes were prepared for hybridization with the mRNAs to be detected. The probes were generated by in-vitro transcription of a PCR product by means of a T7 or SP6 RNA polymerase, using labeled UTPs. The template for the PCR-aided amplification was provided by the above-described plasmid vectors pGEMT-BI1, pGEMT-UBI. Depending on the orientation of the insert, different RNA polymerases were used for generating the antisense strand. T7-RNA polymerase was used for pGEMT-BI1, while SP6—RNA polymerase was used for pGEMT-UBI. The insert of the individual vector was amplified via PCR using flanking standard primers (M13 fwd and rev). The reaction proceeded with the following end concentrations in a total volume of 50 μl of PCR buffer (Silverstar):

| | |
|---|---|
| M13-fwd: | 5′-GTAAAACGACGGCCAGTG-3′ |
| M13-rev: | 5′-GGAAACAGCTATGACCATG-3′ |

10% dimethyl sulfoxide (v/v)
2 ng/μl of each primer (M13 forward and reversed)
1.5 mM MgCl₂,
0.2 mM dNTPs,
4 units Taq polymerase (Silverstar),
2 ng/μl plasmid DNA.

The amplification was carried out in a Thermocycler (Perkin-Elmar 2400) with the following temperature program: 94° C. for 3 minutes; 30 cycles of 30 seconds at 94° C.; 30 seconds at 58° C.; 1.2 minutes at 72° C.; 72° C. for 5 minutes; then cooling at 4° C. until further use. The success of the reaction was verified in a 1% strength agarose gel. The products were subsequently purified using a “High Pure PCR-Product Purification Kit” (Boehringer-Mannheim). This gave approximately 40 μl of column eluate, which was again verified in the gel and stored at −20° C.

The RNA polymerization, the hybridization and the immuno-detection were carried out largely following the kit manufacturer's instructions regarding the nonradioactive RNA detection (DIG System User's Guide, DIG-Luminescence detection Kit, Boehringer-Mannheim, Kogel et al. (1994) Plant Physiol 106:1264-1277). 4 μl of purified PCR product were treated with 2 μl of transcription buffer, 2 μl of NTP labeling mix, 2 μl of NTP mix and 10 μl of DEPC water. Then, 2 μl of the T7 RNA polymerase solution were pipetted in. The reaction was then carried out for 2 hours at 37° C. and then made up to 100 μl with DEPC water. The RNA probe was detected in an ethidium bromide gel and stored at −20° C.

To prepare the hybridization, the membranes were first agitated gently for 1 hour at 68° C. in 2×SSC (salt, sodium citrate), 0.1% SDS buffer (sodium dodecyl sulfate), the buffer being renewed twice or 3 times. The membranes were subsequently applied to the internal wall of hybridization tubes preheated at 68° C. and incubated for 30 minutes with 10 ml of Dig-Easy hybridization buffer in a preheated hybridization oven. In the meantime, 10 μl of probe solution were denatured for 5 minutes at 94° C. in 80 μl of hybridization buffer, and the mixture was subsequently placed on ice and centrifuged briefly. For the hybridization, the probe was then transferred into 10 ml of hybridization buffer at a temperature of 68° C., and the buffer in the hybridization tube was replaced by this probe buffer. Hybridization was then carried out overnight, likewise at 68° C. Prior to the immunodetection of RNA-RNA hybrids, the blots were washed twice under stringent conditions for in each case 20 minutes in 0.1% (w/v) SDS, 0.1×SSC at 68° C. For the immunodetection, the blots were first agitated gently twice for 5 minutes in 2×SSC, 0.1% SDS at RT. 2 stringent wash steps were subsequently carried out for in each case 15 minutes at 68° C. in 0.1×SSC, 0.1% SDS. The solution was then replaced by wash buffer without Tween. The reaction mix was shaken for 1 minute and the solution was exchanged for blocking reagent. After a further 30 minutes' shaking, 10 μl of antifluorescein antibody solution were added, and shaking was continued for 60 minutes. This was followed by two 15-minute wash steps in Tween-containing wash buffer. The membrane was subsequently equilibrated for 2 minutes in substrate buffer and, after being left to drain, transferred to a sheet of acetate paper. A mixture of 20 μl CDP-Star™ and 2 ml of substrate buffer was then divided uniformly on the “RNA side” of the membrane. The membrane was subsequently covered with a second sheet of acetate paper and the edges were heat-sealed to provide a water-tight seal, avoiding air bubbles. In a dark room, the membrane was then covered for 10 minutes with an X-ray film and the film was subsequently developed. The exposure time was varied as a function of the intensity of the luminescent reaction.

Unless otherwise specified, the solutions were part of the kit as delivered (DIG-Luminescence detection Kit, Boehringer-Mannheim). All the others were prepared from the following stock solutions by dilution with autoclaved distilled water. Unless otherwise specified, all the stock solutions were made with DEPC (like DEPC water) and subsequently autoclaved.
DEPC water: distilled water is treated overnight at 37° C. with diethyl pyrocarbonate (DEPC, 0.1%, w/v) and subsequently autoclaved.
10×MOPS buffer: 0.2 M MOPS (morpholine-3-propanesulfonic acid), 0.05 M sodium acetate, 0.01 M EDTA, pH brought to 7.0 with 10 M NaOH.
20×SSC (sodium chloride/sodium citrate, salt/sodium citrate): 3 M NaCl, 0.3 M trisodium citrate×2H₂O, pH brought to 7.0 with 4 M HCl.
1% SDS (sodium dodecyl sulfate) sodium dodecyl sulfate (w/v), without DEPC.
RNA sample buffer: 760 μl formamide, 260 μl formaldehyde, 100 μl ethidium bromide (10 mg/ml), 80 μl glycerol, 80 μl bromophenol blue (saturated), 160 μl 10×MOPS, 100 μL water.
10×wash buffer without Tween: 1.0 M maleic acid, 1.5 M NaCl; without DEPC, bring to pH 7.5 with NaOH (solid, approx. 77 g) and 10 M NaOH.
Tween-containing wash buffer: made by adding Tween to wash buffer without Tween (0.3%, v/v).
10×blocking reagent: suspend 50 g of blocking powder (Boehringer-Mannheim) in 500 ml of wash buffer without Tween.
Substrate buffer: bring 100 mM Tris (trishydroxymethylaminomethane), 150 mM NaCl to pH 9.5 with 4 M HCl.
10×color marker: 50% glycerol (v/v), 1.0 mM EDTA pH 8.0, 0.25% bromophenol blue (w/v), 0.25% xylene cyanol (w/v).

A BI1 expression was analyzed as described using RT-PCR and cDNA gel blots and revealed that BI1 is predominantly expressed in the mesophyll tissue of leaves, while ubiquitin is constitutively expressed uniformly in epidermis and mesophyll (FIG. 8).

Expression of BI1 as response to treatment of the plants with toxic culture filtrates of Bipolaris sorokiniana can furthermore be observed. Barley primary leaves show typical necrotic lesions (leaf spot blotch symptoms) after treatment of the plants with toxic culture filtrates of Bipolaris sorokiniana (procedure as described by Kumar et al. 2001). The leaf necroses were discernible 48 hours post-treatment. The tissue damage observed was more pronounced in the Bgh-resistant line BcIngrid-mlo5 (122) than in the parent line Ingrid (Mlo genotype, Kumar et al. 2001). 72 hours post-treatment (hat), the expression of BI1 correlates with the manifestation of the leaf necroses (FIG. 12).

### Example 7

The wheat oxalate oxidase promoter (germin 9f-2.8) is employed to obtain stable, mesophyll-specific overexpression. In barley, the corresponding oxalate oxidase expression is mesophyll-specific, weakly constitutive and pathogen-responsive (Gregersen P L et al. (1997) Physiol Mol Plant Pathol 51: 85-97). It can therefore be utilized for the mesophyll-specific expression of BI1. As a control, HvBI1 is overexpressed under the control of the maize ubiquitin promoter (Christensen A H et al. (1992) Plant Mol Biol 18:675-689) or of the rice actin promoter (Zhang W et al. (1991) Plant Cell 3:1155-1165). The following constructs are employed:
a) pUbiBI-1 (SEQ ID NO: 33; for the transient transformation of barley and wheat by means of particle bombardment. Expression of BI-1 under the control of the maize ubiquitin promoter).
b) pLo114UbiBI-1 (SEQ ID NO: 34; obtained by recloning the Ubi/BI-1 expression cassette as EcoR1 fragment from pUbiBI-1 in pLo114-GUS-Kan; binary vector for the transient transformation of barley with A. tumefaciens)
c) pOXoBI-1 (SEQ ID NO: 35; mesophyll-specific TaGermin 9f-2.8 promoter upstream of BI1 for the transformation of wheat via particle bombardment.
d) pLo1140XoBI-1 (SEQ ID NO: 36)

Wild-type barley, wheat and mlo barley are transformed, propagated and selfed. The transformation of barley and wheat proceeds as described (Repellin A et al. (2001) Plant Cell, Tissue and Organ Culture 64: 159-183): To this end, calli from immature wheat (or barley) embryos are transformed via biolistic gene transfer with microprojectiles. pUC-based vectors together with vectors which bear selection markers are cotransformed here. Thereafter, the embryos are grown on selection medium and regenerated. Barley is transformed with the aid of Agrobacterium tumefaciens. A binary vector based on pCambia_{—}1301 is employed for this purpose. Immature barley embryos are cocultured with A. tumefaciens, selected and subsequently regenerated (Repellin A et al. (2001) Plant Cell, Tissue and Organ Culture 64: 159-183; Horvath H et al. (2003) Proc Natl. Acad Sci USA 100: 365-369; Horvath H et al. (2002) in Barley Science, eds. Slafer, G. A., Molina-Cano, J. L., Savin, R., Araus, J. L. & Romagosa, J. (Harworth, New York), pp. 143-176; Tingay S et al. (1997) Plant J. 11: 1369-1376).

The transgenic (recombinant) barley and wheat plants of the T1 or T2 generation are studied for resistance to hemibiotrophic and perthotrophic pathogens. To this end, the leaves are inoculated with a variety of pathogens. The biotrophic pathogens used are powdery mildew of barley (Blumeria graminis f.sp. hordei) and leaf rust (Puccinia hordei). As a measure for the susceptibility to mildew, the number of pustules per unit leaf area is evaluated 5-7 days after inoculation with 2-5 conidia per mm² of leaf area (Beβer K et al. (2000) Mol Plant Pathology 1: 277-286). Bipolaris sorokiniana and Magnaporthe grisea are used as hemibiotrophic pathogens. Inoculation is as described above (Kumar J et al. (2001) Phytopathology 91: 127-133; Jarosch B et al. (1999) Mol Plant Microbe Inter 12: 508-514). The number and size of the leaf lesions 2 to 6 days after spray inoculation with conidia is used as measure for the susceptibility (Kumar J et al. (2001) Phytopathology 91:127-133; Jarosch B et al. (1999) Mol Plant Microbe Inter 12:508-514; Jarosch B et al. (2003) Mol Plant Microbe Inter 16:107-114.). Fusarium graminearum is used as perthotrophic pathogen.

To determine the fusarium head blight (FHB) type-I resistance, wheat ears in an early stage of flowering are sprayed with a macroconidial suspension (approx. 2×10⁵ ml⁻¹) of Fusarium graminearum and of Fusarium culmorum, respectively. The inoculated plants are transferred for 3 days into a humid chamber with an air temperature of 25° C. and a relative atmospheric humidity of 100%. Thereafter, the plants are incubated in the greenhouse under continuous light at a temperature of 20° C., and the severity of the FHB symptoms along the ear are evaluated after 5, 7 and 8 days.

To quantify the fusarium head blight (FBH) type-II resistance, in each case 10-20 μl aliquots of a macroconidial suspension (approx. 2×10⁵ ml⁻¹) of Fusarium graminearum and Fusarium culmorum, respectively, are injected into individual, relatively centrally located spikelets of wheat plants. The inoculated plants are transferred for 3 days into a humid chamber at an air temperature of 25° C. and a relative atmospheric humidity of 100%. Thereafter, the plants are incubated in the greenhouse under continuous light at a temperature of 20° C., and the spreading of the FHB symptoms along the ear is evaluated after 7, 14 and 21 days. The spreading of the symptoms along the ear (what is known as Fusarium spreading) is taken as a measure for the FHB type-II resistance.

### Comparative Example 1

### Transient BI1 Expression in the Epidermis, and Evaluation of the Development of the Fungal Pathogen

Barley cv Ingrid leaf segments were transformed with a pGY-BI1 together with a GFP expression vector. Thereafter, the leaves were inoculated with Bgh, and the result was analyzed after 48 hours by means of light and fluorescent microscopy. The penetration in GFP-expressing cells was assessed by detecting haustoria in live cells and by assessing the fungal development in precisely those cells. A transient transformation method which had already been described for the biolistic introduction of DNA and RNA into epidermal cells of barley leaves was employed (Schweizer P et al. (1999) Mol Plant Microbe Interact 12:647-54; Schweizer P et al. (2000) Plant J 2000 24:895-903).

To prepare the microcarriers, 55 mg of tungsten particles (M 17, diameter 1.1 μm; Bio-Rad, Munich) were washed twice with 1 ml of autoclaved distilled water and once with 1 ml of absolute ethanol, dried and taken up in 1 ml of 50% strength glycerol (approx. 50 mg/ml stock solution). The solution was diluted to 25 mg/ml with 50% strength glycerol, mixed thoroughly prior to use, and suspended in an ultrasonic bath.

To coat the microcarriers for each bombardment, 0.3 μg of plasmid pGFP (GFP under the control of the CaMV 35S promoter; Schweizer P et al. (1999) Mol Plant-Microbe Interact 12:647-654.), 0.7 μg of blank vector pGY or pGY-BI1 (1 μl), 12.5 μl of tungsten particle suspension (25 mg/ml; corresponding to 312 μg of tungsten particles), 12.5 μl of 1 M Ca(NO₃)₂ solution (pH 10) were combined dropwise with constant mixing, the mixture was left to stand for 10 minutes at RT and then briefly centrifuged, and 20 μl of the supernatant were drawn off. The remainder with the tungsten particles is resuspended (ultrasonic bath) and employed in the experiment.

Segments (approx. 4 cm in length) of barley primary leaves were used. The tissue was placed on 0.5% Phytagar (GibcoBRL™ Life Technologies™, Karlsruhe) supplemented with 20 μg/ml benzimidazole in Petri dishes (diameter 6.5 cm), and the edges were covered directly prior to particle bombardment with a stencil provided with a rectangular opening of 2.2 cm×2.3 cm. One after the other, the dishes were placed on the bottom of the vacuum chamber (Schweizer P et al. (1999) Mol Plant Microbe Interact 12:647-54) over which a nylon mesh (mesh size 0.2 mm, Millipore, Eschborn) on an apertured plate had been inserted (5 cm above the bottom, 11 cm underneath the macrocarrier, see hereinbelow) to act as diffuser in order to disperse particle aggregates and to slow down the particle stream. For each bombardment, the macrocarrier (plastic sterile filter holder, 13 mm, Gelman Sciences, Swinney, UK), which was attached at the top of the chamber, was loaded with 5.8 μl of DNA-coated tungsten particles (microcarrier, see hereinbelow). The pressure in the chamber was reduced by 0.9 bar using a diaphragm vacuum pump (Vacuubrand, Wertheim), and the surface of the plant tissue was bombarded with the tungsten particles at a helium gas pressure of 9 bar. The chamber was aerated immediately thereafter. To label transformed cells, the leaves were bombarded with the plasmid (pGFP; vector pUC18-based, CaMV 35S promoter/terminator cassette with inserted GFP gene; Schweizer P et al. (1999) Mol Plant Microbe Interact 12:647-54; provided by Dr. P. Schweizer, Institut für Pflanzengenetik [Department of Plant Genetics] IPK, Gatersleben, Germany). Each time before another plasmid was used for the bombardment, the macrocarrier was cleaned thoroughly with water. Following incubation for four hours after the bombardment with slightly open Petri dishes, RT and daylight, the leaves were inoculated with 100 conidia/mm² of the barley powdery mildew fungus (race A6; Blumeria graminis f. sp. hordei mildew A6) and incubated for a further 40 hours under identical conditions. The penetration was then evaluated. The result (for example the penetration efficiency), defined as percentage of attacked cells with a mature haustorium and a secondary hypha (secondary elongating hyphae) was analyzed by fluorescence and light microscopy. Inoculation with 150 conidia/mm² results in an attack frequency of approximately 50% of the transformed cells. A minimum of 100 interaction sites were evaluated for each individual experiment. Transformed (GFP-expressing) cells were identified under excitation with blue light. Three different categories of transformed cells were distinguished:
1. Penetrated cells comprising a readily recognizable haustorium. A cell with more than one haustorium counted as one cell.
2. Cells which were attacked by a fungal appressorium, but comprise no haustorium. A cell which was attacked repeatedly by Bgh, but comprises no haustorium, counted as one cell.
3. Cells which are rot attacked by Bgh.

Stomatal cells and subsidiary stomatal cells were excluded from the evaluation. Surface structures of Bgh were analyzed by light microscopy or fluorescent staining of the fungus with 0.3% Calcofluor (w/v in water) for 30 sec. Fungal development can be evaluated readily by staining with Calcofluor followed by fluorescence microscopy. While the fungus develops a primary germ tube and an appressorial germ tube in cells transformed with BI1-dsRNA, it fails to develop a haustorium. The development of haustoria is a precondition for the formation of a secondary hypha.
The penetration efficiencies (penetration rates) are calculated as the number of penetrated cells divided by the number of the attacked cells, multiplied by 100.

The penetration efficiency is used for determining the susceptibility of cells which are transfected with pGY-BI1 in comparison with cells which are transformed with a blank vector control (FIG. 10). It can be seen that the overexpression of BI1 significantly increases the penetration frequency of Bgh (FIG. 10). In six independent experiments, overexpression in the susceptible barley variety Ingrid brought about a significant increase in the average penetration efficiency (PE) from 47% to 72% (165% of the controls) in cells which overexpress BI1 in comparison with cells which were transformed with blank vector (control) (FIG. 10).

Furthermore, epidermal cells of the Bgh-resistant m1o5 barley were transformed transiently as described above with the BI1 overexpression construct pGY-1. The mlo5 genotype in a Pallas or Ingrid background shows minor susceptibility to Bgh. In 7 independent experiments, a penetration efficiency of a minimum of 0 to a maximum of 11% was found in control plants (transformation with blank vector and GFP vector). Surprisingly, BI1 overexpression (pGY-BI1) resulted in a virtually complete reconstitution of the susceptible phenotype, i.e. the mlo resistance was broken almost completely. The average penetration efficiency of Bgh on Ingrid-mlo5 and Pallas-mlo5 leaf segments climbs from 4% to 23% and from 6% to 33%, respectively (FIG. 11). This means a relative increase in penetration to 520% and 510%, respectively, of the controls. Moreover, the overexpression of BI1 in barley cv Manchuria increased the susceptibility to the wheat pathogen Blumeria graminis f.sp. tritici from 0 to 4% to 19 to 27% in three independent experiments (FIG. 13).

1. A method for generating or increasing the resistance, in plants, to at least one biotic or abiotic stress factor, comprising the following steps:
a) increasing the amount of protein, or the function, of at least one Bax inhibitor-1 (BI1) protein in at least one plant tissue with the proviso that the expression in the leaf epidermis remains essentially unchanged or is reduced, and
b) selection of the plants in which, in comparison with the starting plant, a resistance to at least one biotic or abiotic stress factor exists or is increased.

2. The method according to claim 1, wherein the stress factor is a plant pathogen.

3. The method according to claim 1, wherein the stress factor is a necrotrophic or hemibiotrophic pathogen.

4. The method according to claim 1, wherein the BI-1 protein comprises at least one sequence which has at least 50% homology with at least one BI1 consensus motif selected from the group consisting of

| | |
|---|---|
| a) H(L/I)KXVY | (SEQ ID NO: 45) |
| b) AXGA(Y/F)XH | (SEQ ID NO: 46) |
| c) NIGG | (SEQ ID NO: 47) |
| d) P(V/P)(Y/F)E(E/Q)(R/Q)KR | (SEQ ID NO: 48) |
| e) (E/Q)G(A/S)S(V/I)GPL | (SEQ ID NO: 49) |
| f) DP(S/G)(L/I)(I/L) | (SEQ ID NO: 50) |
| g) V(G/A)T(A/S)(L/I)AF(A/G)CF(S/T) | (SEQ ID NO: 51) |
| h) YL(Y/F)LGG, | (SEQ ID NO: 52) |
| preferably EYLYLGG | (SEQ ID NO: 53) |
| i) L(L/V)SS(G/W)L(S/T)(I/M)L(L/M)W | (SEQ ID NO: 54) |
| j) DTGX(I/V)(I/V)E. | (SEQ ID NO: 55) |

5. The method according to claim 1, wherein the BI-1 protein is encoded by a polypeptide comprising at least one sequence selected from the group consisting of:
a) the sequences as shown in SEQ ID NO: 2, 4, 6, 8, 10, 12, 14, 16, 18, 20, 22, 24, 26, 28, 30, 32 and 38, and
b) sequences which have at least 50% identity with one of the sequences as shown in SEQ ID NO: 2, 4, 6, 8, 10, 12, 14, 16, 18, 20, 22, 24, 26, 28, 30, 32 and 38,
c) sequences which comprise at least one part-sequence of at least 10 contiguous amino acid residues of one of the sequences as shown in SEQ ID NO: 2, 4, 6, 8, 10, 12, 14, 16, 18, 20, 22, 24, 26, 28, 30, 32 and 38.

6. The method according to claim 1, wherein the increase in the protein quantity or function of at least one BI1 protein is effected by recombinant expression of said BI1 protein under the control of a root-, tuber- or mesophyll-specific promoter.

7. The method according to claim 1, comprising
(a) stably transforming a plant cell with a recombinant expression cassette comprising a nucleic acid sequence coding for a BI protein in functional linkage with a tissue-specific promoter, the promoter having essentially no activity in the leaf epidermis and the promoter being heterologous with regard to said nucleic acid sequence which codes for the BI protein;
(b) regenerating the plant from the plant cell; and
(c) expressing said nucleic acid sequence which codes for a BI protein in an amount and for a period sufficient to generate or to increase a stress and/or pathogen resistance in said plant.

8. The method according to claim 1, wherein the plant is selected from among the monocotyledonous and dicotyledonous plants.

9. The method according to claim 1, wherein the plant is selected from the group of the monocotyledonous plants consisting of wheat, oats, millet, barley, rye, maize, rice, buckwheat, sorghum, triticale, spelt, linseed and sugar cane.

10. The method according to claim 1, wherein the expression of the Bax inhibitor-1 (BI-1) in the mesophyll is increased.

11. The method according to claim 1, wherein the plant has an mlo-resistant phenotype, or the expression or function of MLO, RacB and/or NaOx is inhibited or, in comparison with a control plant, is reduced at least in the epidermis and/or the expression or function of PEN2, SNAP34 and/or PEN1 is increased at least in the epidermis in comparison with a control plant.

12. A polypeptide sequence coding for a BI1 protein comprising at least one sequence selected from the group consisting of
a) the sequences as shown in SEQ ID NO: 12, 14, 16, 18, 20, 22, 24, 28, 30, 32 or 38,
b) sequences which have at least 90%, preferably at least 95%, especially preferably at least 98%, homology with one of the sequences as shown in SEQ ID NO: 12, 14, 16, 18, 20, 22, 24, 28, 30, 32 or 38, and
c) sequences which comprise at least 10, preferably at least 20, especially preferably at least 30, contiguous amino acids of one of the sequences as shown in SEQ ID NO: 12, 14, 16, 18, 20, 22, 24, 28, 30, 32 or 38.

13. A nucleic acid sequence coding for a polypeptide sequence according to claim 12.

14. A recombinant expression cassette comprising a nucleic acid sequence coding for a BI protein in functional linkage with a tissue-specific promoter, the promoter having essentially no activity in the leaf epidermis and the promoter being heterologous with regard to said nucleic acid sequence which codes for the BI protein.

15. The recombinant expression cassette comprising a nucleic acid sequence coding for a BI protein in functional linkage with a tissue-specific promoter, the promoter having essentially no activity in the leaf epidermis and the promoter being heterologous with regard to said nucleic acid sequence which codes for the BI protein, where
a) the BI1 protein is as defined in claim 4, and/or
b) the tissue-specific promoter is selected from the group of the root-, tuber- or mesophyll-specific promoters.

16. A recombinant vector comprising an expression cassette according to claim 14.

17. A recombinant organism comprising at least one expression cassette according to claim 14.

18. The recombinant organism according to claim 17 selected from the group consisting of bacteria, yeasts, nonhuman animals and plants.

19. The recombinant organism according to claim 17 or 18, selected from the group of the plants consisting of wheat, oats, millet, barley, rye, maize, rice, buckwheat, sorghum, triticale, spelt, linseed, sugar cane, oilseed rape, cress, Arabidopsis, cabbage species, soybean, alfalfa, pea, beans, peanut, potato, tobacco, tomato, eggplant, paprika, sunflower, Tagetes, lettuce, Calendula, melon, pumpkin/squash and zucchini.

20. The recombinant organism according to claim 17, wherein the organism is a plant which additionally has an mlo-resistant phenotype.

21. The recombinant expression cassette comprising a nucleic acid sequence coding for a BI protein in functional linkage with a tissue-specific promoter, the promoter having essentially no activity in the leaf epidermis and the promoter being heterologous with regard to said nucleic acid sequence which codes for the BI protein, where
a) the BI1 protein is as defined in claim 5, and/or
b) the tissue-specific promoter is selected from the group of the root-, tuber- or mesophyll-specific promoters.
